(19) United States
(12) Patent Application Publication
(10) Pub. No.: US 2023/0383065 A1
(43) Pub. Date: Nov. 30, 2023

(54) COMPOSITIONS COMPRISING BIODEGRADABLE POLYMERS

(71) Applicant: LANDA LABS (2012) LTD., Rehovot (IL)

(72) Inventors: Benzion LANDA, Nes Ziona (IL); Sagi ABRAMOVICH, Ra'anana (IL); Galia GOLODETZ, Rehovot (IL); Jose KUPERWASSER, Ashdod (IL)

(73) Assignee: LANDA LABS (2012) LTD., Rehovot (IL)

(21) Appl. No.: 18/361,965

(22) Filed: Jul. 31, 2023

Related U.S. Application Data
(63) Continuation-in-part of application No. PCT/IB22/50826, filed on Jan. 31, 2022.

(30) Foreign Application Priority Data
Feb. 1, 2021 (GB) 2101379.2

(51) Int. Cl. C08J 3/20; C08K 3/22

(52) U.S. Cl. CPC C08J 3/203; C08K 3/22; C08K 2201/005; C08K 2201/011; C08K 2003/2241; C08J 2300/16

(57) ABSTRACT

Disclosed are compositions comprising macroparticles of swelled polymer matrix, the macroparticles comprising a thermoplastic biodegradable polymer swelled with at least one non-aqueous polar swelling agent, the macroparticles further comprising a plurality of pigment particles dispersed and embedded therein. The pigment particles may provide a color and/or a radiation protective effect to the composition. Methods of preparation and uses of such compositions are also provided.

### CROSS-RELATED APPLICATIONS

The present application is a Continuation-in-Part application of International Application No. PCT/IB2022/050823, filed on Jan. 31, 2022, which claims Paris Convention priority from Great-Britain Patent Application GB 2101379.2, filed on Feb. 1, 2021. The entire contents of the afore-mentioned applications are incorporated herein by reference for all purposes as if fully set forth herein.

### FIELD

The disclosure relates to compositions comprising pigments or agents protecting from ultraviolet (UV) radiation. Methods for preparing the same and uses thereof, in industries ranging from cosmetics to coatings, are also provided.

### BACKGROUND

Pigments are widely used in fields spanning from industrial applications, such as coatings, paints or inks, to more personal uses, such as in cosmetics. In the cosmetic industry, pigments are primarily used to provide a desired color to skin, nails and hair, and to related personal-care products (e.g., foundation, eye shadows, lipstick, mascara, nail varnish, etc.). Pigments may additionally, or alternatively, serve as ultraviolet (UV)-protective agents, scattering, filtering or blocking such radiation in sunscreen products, among others. The coloring effect is typically provided by particles in the low micrometer (μm) range of no more than 100 μm, whereas the UV-protective effect can also be afforded in the sub-micron range, even in the nanometer (nm) range.

UV radiation is ubiquitous, the sun being the most common source of UV radiation although not the only source. As UV radiation can cause damage to people, animals and objects, compositions that provide protection from UV radiation are useful.

In the biological context, UV-protective compositions, i.e. compositions that reduce or block the transmission of UV rays, generally either by scattering or absorbing such rays, are commonly employed to protect against sunburn. Sunburn is a form of radiation burn resulting from an overexposure to UV radiation typically from the sun, but also from artificial sources, such as tanning lamps, welding arcs, and ultraviolet germicidal irradiation.

Normal symptoms of sunburn in humans and other animals include reddening of the skin, general fatigue and mild dizziness. An excess of UV radiation can be life-threatening in extreme cases. Excessive UV radiation is considered to be the leading cause of non-malignant skin tumors, as well as increasing the risk of certain types of skin cancer.

Sunscreen compositions are commonly used to prevent sunburn and are believed to prevent squamous cell carcinomas and melanomas. Furthermore, they have been reported to delay the development of wrinkles and additional age-related skin conditions.

Specifically, sunscreen compositions are topical compositions that include UV-protecting agents that absorb and/or reflect at least some of the sun's UV radiation. Depending on their mode of action, they are typically classified as chemical or physical sunscreens.

Chemical sunscreen compositions comprise organic compounds that absorb UV radiation to reduce the amount of UV radiation that reaches the skin. Being transparent to visible light and thereby being invisible when applied to the skin, chemical sunscreen compositions are popular for use. However, some organic compounds used in chemical sunscreen compositions have been found to generate free radicals which can cause skin damage, irritation and accelerated aging of the skin. Chemical sunscreen compositions may therefore require the addition of a photostabilizer. Such concerns fade for industrial products and organic UV-absorbers are used in coatings, such as in transparent wood varnishes.

Physical sunscreen compositions reflect and absorb UV radiation. Known physical sunscreen compositions comprise particles of inorganic materials, mainly titanium oxide and/or zinc oxide. In order to obtain absorption and/or reflection of ultraviolet radiation over the full UVA and UVB range, relatively large particles are used. Due to the large particle size, such sunscreen compositions are viscous and opaque and tend to leave a white cast on the skin.

Many sunscreen compositions protect against UV radiation in the 280-315 nanometer (nm) range (UVB radiation) that causes sunburn, but do not protect against UV radiation in the 315-400 nm range (UVA radiation), which does not primarily cause sunburn but can increase the rate of melanoma and photodermatitis, and is furthermore generally associated with skin aging.

It is generally preferred that sunscreen compositions are transparent on the skin. In order for physical sunscreen compositions to be transparent, the particles of inorganic material should be in the form of nanoparticles, which absorb and/or scatter UV light but not visible light, rendering them substantially transparent on the skin. However, use of nanoparticles reduces the range of wavelengths absorbed by the inorganic materials. Some known sunscreen compositions therefore block both UVA and UVB radiation by use of a combination of different UV-absorbing or scattering materials, generally termed UV-protecting agents, each of which blocks radiation over a limited range of the UV spectrum.

Similarly, UV-protective compositions can benefit inert materials or objects that may be negatively affected by UV radiation. For instance, UV radiation can reduce the life-span of materials (e.g., natural and synthetic polymers), and may modify colors of objects, especially in articles that are subjected to prolonged sun exposure, such as buildings or vehicles.

Various articles' coatings are known to provide protection against UV radiation damage by blocking or reducing transmission of UV rays. Use of such coatings may in turn reduce the detrimental effect of UV radiation on a living animal. For example, use of said coating on optical lenses, thereby reducing the transmission of UV radiation, may reduce the incidence of UV-induced optical disorders such as cataract. Materials serving for the fabrication of windows incorporating or coated with suitable UV-protecting agents may reduce the transmission of UV radiation to subjects, plants, surfaces or objects shielded by such windows.

The Applicant has previously reported in WO 2016/151537, WO 2017/175164 and WO 2017/191585, compositions comprising doped or undoped inorganic nanoparticles having alone a relatively broad absorption in the UV range, capable in turn to provide a wide UV-protective effect to living subjects and surfaces of inanimate objects.

While grinding materials to size range of nanoparticles (e.g., having a primary particle size of 100 nanometer or less) may provide for UV-protective agents having a surprisingly broad effect, it creates a few challenges readily appreciated by persons working with extremely fine particles. Formulatory challenges, for instance, include the dispersion of the nanoparticles and their stabilization in dispersed form, as they have a tendency to agglomerate. Leaving this issue unresolved impairs, among other things, the intended function of the particles, the lifespan of the product and the compliance to use it. There are also increasing regulatory concerns regarding the safety of such nanoparticles when applied to a living subject (or to a surface coming in contact therewith).

One way to avoid such risks is to embed the nanoparticles in a polymeric matrix, reducing their ability to aggregate and migrate out. Cosmetic compositions comprising pigment particles in a polymeric matrix are described in EP 2229931 by Dainichiseika Color Chem., wherein pigment nanoparticles are encapsulated in spheres of (meth)acrylic resin, prepared by suspension polymerization in an aqueous medium. Another way of embedding particles in a polymeric matrix involves dissolving the polymer in a solvent, mixing the obtained solution with the desired nanoparticles, followed by elimination of the solvent, as described, e.g., in WO 2019/167749 by Toray Industries. The drying of the dissolved polymer when applied to the skin is expected to yield a continuous thin film of the polymer, creating a sealing effect (i.e., the skin cannot “breathe”).

In WO 2017/013633, and in contrast with the foregoing methods, the Applicant elected embedding the nanoparticles in a different set of polymers, the matrix being accordingly prepared by alternative methods and being additionally differently shaped as flakes to provide an enhanced contact area with the surface coated thereby, improving the feel and persistence of the polymer matrix on the surface, yielding a superior UV-protective effect and avoiding a sealing effect.

Polymers conventionally used for immobilization of particles therein are designed to subsist for long periods of time, so as to properly serve their intended role. In cosmetic products designed to be applied, for instance, to human skin or hair, the pieces of embedding polymers would be regularly washed away and may end up in the environment, where they may remain for many years as microplastic waste. The widespread presence of microplastics in practically all ecosystems, including continental and oceanic waters, sediments, air and soils, has become a global environmental concern. Therefore, an approach intended to resolve a regulatory concern with respect to fine particles of pigments and/or UV-protective agents, may in turn create an environmental issue.

It would be desirable to have compositions comprising microparticles or nanoparticles of pigments and/or UV-protective agents, where such fine particles are embedded in polymers maintaining their effectiveness with respect to their intended use, while balancing the need for immediate safety and long-term environmental issues. Such compositions would be advantageous inter alia for cosmetic and/or UV-protective products.

### SUMMARY

With a view to mitigating at least some of the foregoing disadvantages, the Inventors suggest using biodegradable polymers, which would degrade relatively rapidly in the environment, e.g., by biological processes, such as enzymatic mechanisms (carried out by bacteria, fungi, algae etc.) or by nonenzymatic processes, such as chemical hydrolysis. While such polymers have been used on or in living subjects, for instance, for controlled release of drugs, this is typically contrary to the presently sought effect of immobilization. Moreover, biodegradable polymers suitable for the present teachings need additionally be thermoplastic and swellable in view of a preferred method of manufacturing of polymer matrix elements (also termed macroparticles), as further detailed herein.

This selection of particular polymers presents new challenges since biodegradable polymers tend to have a higher permeability to water than their non-biodegradable counterparts, in order to permit the natural degradation of the former by hydrolysis. Thus, while the relatively more hydrophilic nature of biodegradable polymers would facilitate their swelling with aqueous liquids, as compared to regular polymers such as disclosed in WO 2017/013633, this might accelerate their degradation to an extent incompatible with the preparation of macroparticles having a sufficient shelf-life to maintain the pigment particles as desirably dispersed. Free pigment particles may need be avoided in a number of circumstances.

Thus, in order to postpone the biodegradation of the matrix elements until after the pigment particles have served their intended use while immobilized in dispersed form in the macroparticles, it would be preferred to use a non-aqueous swelling liquid. A person having ordinary skill in the pertinent field can readily appreciate that the more polar nature of the biodegradable polymers renders them less compatible with non-polar liquids, such as oils disclosed in U.S. Pat. No. 10,617,610 for the swelling of regular, non-biodegradable polymers. But trying to overcome this problem by combining a relatively polar biodegradable polymer with a relatively polar liquid may in turn increase the likelihood of solubilizing/dissolving the polymer, instead of swelling it. As explained hereinbelow in more details, the biodegradable polymer is not to be solubilized in a solvent, but on the contrary swelled to form a polymer matrix capable of entrapping particles.

The swelling of the biodegradable polymers is believed to space away the polymer chains, making room for the particles to be entrapped therebetween. Moreover, the presence of a suitable swelling agent is expected to sufficiently soften the polymers so as to render the polymer matrix malleable, kneadable and pulverizable with relatively low mechanical energy. Importantly, the resulting swollen granules or powder can be formed without melting the polymer. The morsels of swollen polymer matrix can then be mixed with the materials desired for entrapment and comminuted and/or shaped to form the macroparticles (e.g., flakes) of the present composition, particulate materials being advantageously evenly dispersed within the swollen polymer in the process.

The dispersion of particles (e.g., pigments) in a polymeric matrix leads to their relative immobilization, the swelling agent once adsorbed by the polymer not enabling any significant Brownian motion, in contrast with the relative motility such particles would have in a suspension. As used herein, the terms “embedded”, “entrapped”, “immobilized” and their grammatical variants can be interchangeably used to refer to this particular type of dispersion wherein the particles are substantially fixed and enclosed in a surrounding matrix. Advantageously, the particles are dispersed as individual particles or discrete clusters thereof.

According to an aspect of the present disclosure, there is provided a composition comprising: a) macroparticles of swelled biodegradable polymer matrix comprising a thermoplastic biodegradable polymer swelled with at least one non-aqueous polar swelling agent having a calculated Hydrophilic-Lipophilic Balance (HLB) value of at least about 3; and b) a plurality of pigment particles, wherein the pigment particles are discretely and individually dispersed and embedded in the polymer matrix macroparticles.

In some embodiments, the pigment particles do not substantially migrate out of the swelled biodegradable polymer matrix macroparticles in which they are dispersed and embedded, so that the composition is substantially devoid of free pigment particles that are non-embedded in the macroparticles. In some embodiments, the ratio between a number of free non-embedded pigment particles and a number of polymer macroparticles (in which pigment particles are embedded), as can be observed by suitable microscopic analysis is at most 10:1, at most 5:1, a most 2:1, at most 1:1, at most 1:2, at most 1:5, at most 1:10, or at most 1:20 per unit area of the field of view microscopically captured.

In some embodiments, the thermoplastic biodegradable polymer is an oil-swellable thermoplastic homo- or co-polymer, optionally, but not necessarily, clear, transparent and/or colorless. In view of their intended use and/or method of preparation, a thermoplastic biodegradable polymer suitable for the present invention is advantageously relatively solid at room temperature (circa 25° C.) and up to body temperatures (e.g., circa 37° C. for human subjects) or to which an inanimate object coated with the present compositions can be exposed (e.g., up to about 60° C.). Such preferences are extended to the swelled polymer matrix, which further takes into account the swelling agent and its relative amount, or the presence of any other material affecting the thermal behavior of the product. As can be appreciated by persons skilled in the field of polymers, as the swellable or the swelled biodegradable polymer are thermoplastic materials, a “relative solidity” of such materials, or such materials being “relatively solid”, at any particular temperature is referring to the fact that they are not necessarily solid but display a viscoelastic behavior. Without wishing to be bound by any particular theory, such feature of the thermoplastic biodegradable polymer and of the macroparticles of swollen matrix made therefrom should ensure, to the extent necessary, that the macroparticles are relatively non-sticky, facilitating their even distribution in a composition according to the present teachings.

To be suitable, the swellable biodegradable thermoplastic polymer may have at least one of a softening temperature (Ts), a melting temperature (Tm), and a glass transition temperature (Tg) of 200° C. or less. In some embodiments, to be relatively solid at room temperature, the swellable polymer can have at least one of a Tm and a Ts being of 20° C. or more, this preferred lower limit advantageously applying to the swollen polymer, as well as to a swelling mixture comprising it. In some embodiments, the swellable polymer, the swollen polymer matrix, and/or the swelling mixture has at least one of said temperatures (Tm or Ts) being 30° C. or more, 40° C. or more, 50° C. or more, or 60° C. or more. A swellable biodegradable thermoplastic polymer suitable for the present teachings may have at least one of a Tm and a Ts being in a range from 20° C. to 200° C., 30° C. to 190° C., 40° C. to 180° C., or 50° C. to 170° C. A swollen polymer matrix of said polymer and/or a swelling mixture comprising the swelled biodegradable thermoplastic polymer and the pigment particles due to be embedded therein should advantageously each have a least one of a respective Tm and Ts being in similar ranges.

In some embodiments, the biodegradable polymers contain hydrolysable functional groups or enzymatically cleavable sites and can be natural or synthetic polymers, optionally prepared from naturally occurring monomers. Thus, synthetic polymers may be compounds having no natural occurrence and may include polymers artificially synthesized according to naturally occurring counterparts. Suitable biodegradable polymers can be selected from polyesters, polyamides, polyurethanes, polyureas, poly(amide-enamine)s, polyanhydrides, polysaccharides, polypeptides, resins, enantiomers thereof, copolymers thereof and combinations thereof. In particular embodiments, the biodegradable swellable polymer alone or in combination is poly(lactic acid) (PLA), poly(lactic-co-glycolic acid) (PLGA), polyhydroxybutyrate (PHB), poly(3-hydroxybutyrate-co-3-hydroxyvalerate) (PHBV), polycaprolactone (PCL), poly(butylene succinate) (PBS), poly(butylene succinate-co-adipate) (PBSA), shellac, or gum rosin.

In one embodiment, the biodegradable polymer is PLA. In another embodiment, the biodegradable polymer is PLGA. In yet another embodiment, the biodegradable polymer is PHB. In yet a further embodiment, the biodegradable polymer is PHBV. In another embodiment, the biodegradable polymer is PBS. In yet another embodiment, the biodegradable polymer is PBSA. In another embodiment, the biodegradable thermoplastic polymer is a combination of any two of the aforesaid polymers, such as a combination of PLA and PHB or of PLA and PCL.

In some embodiments, the non-aqueous polar swelling agent suitable to swell the thermoplastic biodegradable polymer has a calculated HLB value of 4 or more, 5 or more, or 6 or more. In other embodiments, the non-aqueous polar swelling agent has a calculated HLB value of 25 or less, 20 or less, 15 or less. In some embodiments, the non-aqueous polar swelling agent has a calculated HLB value within a range of 3 to 25, within a range of 3 to 20, within a range of 4 and 20, or within a range of 5 and 15.

Such swelling agents may alternatively, or additionally, be characterized by a polarity index of 25 mN/m or less, 20 mN/m or less or 15 mN/m or less.

In some embodiments, the swelling agent is substantially non-volatile under the conditions (e.g., temperature and/or pressure) set for the swelling of the polymer, and for illustration would be non-volatile at a temperature of at least about 50° C. and a pressure of about 100 kPa. However, this is not essential as some volatility can be tolerated or even desired as further detailed hereinbelow.

In some embodiments, the non-aqueous polar swelling agent suitable for swelling the biodegradable polymer is selected from a group consisting of saturated or unsaturated acid mono or diesters, aromatic alcohols, glycols, polyols, aldehydes, ethers, and combinations thereof.

In particular embodiments, the non-aqueous polar swelling agent is selected from a group consisting of: 2-ethylhexyl lactate, acetyl tributyl citrate, acetyl triethyl hexyl citrate, acetyl triethyl citrate, allyl hexanoate, benzyl alcohol, benzyl benzoate, butyl butyryl lactate, butyl lactate, cinnamon bark oil, dibutyl adipate, dibutyl maleate, dibutyl sebacate, diethyl succinate, diethylene glycol diethyl ether, dimethyl glutarate, dimethyl isosorbide, dimethyl maleate, dimethyl methylglutarate, ethyl lactate, gamma decalactone, lactic acid isoamyl ester, lauryl lactate, 1-menthyl lactate, menthalactone, n-pentyl benzoate, PEG-6 caprylic/capric glycerides, triacetin, triethyl citrate and undecanoic lactone.

In one embodiment, the pigment particles are organic or inorganic pigments adapted to provide at least a coloring effect. In another embodiment, the pigment particles are organic or inorganic pigments adapted to provide at least a UV-protective effect, and as such may be also referred to as UV-protective agents. The two properties need not be mutually exclusive, as some pigments may provide both a coloring and a UV-protective effect.

Nevertheless, the predominance of one of the two effects can be associated with the size of the pigment particles, relatively larger particles being able to absorb radiation in the visible range of the spectrum, and relatively smaller particles being able to absorb radiation in the UV range. Therefore, pigment particles adapted to provide at least a coloring effect are typically microparticles, whereas pigment particles adapted to provide at least or only a UV-protective effect are typically nanoparticles. Microparticles of pigments providing at least a coloring effect can additionally provide a UV-protective effect by scattering UV-radiation, whereas nanoparticles of pigments providing at least a UV-protective effect can additionally provide a coloring effect, for instance in view of their actual size and/or chemical identity. Examples of pigments which may provide a shade to a composition even if dispersed as nanoparticles, include bismuth vanadate, bismuth titanate, cerium oxide, cobalt oxide, copper oxide, iron oxide and manganese ferrite, to name a few. The intensity of the coloring imparted by such nanoparticles, otherwise known to provide at least a UV-protective effect, may further depend on their concentration in the composition.

Despite the overlap that may exist in the effects afforded by the different pigment particles, at least in view of their size which may provide for a different predominant effect, it can generally be assumed that nanoparticles of pigments would be suited for the preparation of relatively clear or transparent compositions, whereas microparticles of pigments would be suited for the preparation of relatively opaque compositions.

In some embodiments, the pigment particles, adapted to provide at least a coloring effect are pigment microparticles, wherein at least 50%, at least 55%, at least 60%, at least 65%, at least 70%, at least 75%, at least 80%, at least 85%, at least 90%, at least 95%, at least 97.5%, or at least 99%, per volume of the pigment microparticles, have a longest dimension (e.g., a length) of up to about 5 μm, up to about 3 μm, or up to about 1 μm. In some embodiments, at least 50%, at least 55%, at least 60%, at least 65%, at least 70%, at least 75%, at least 80%, at least 85%, at least 90%, at least 95%, at least 97.5%, or at least 99%, per volume of the pigment microparticles, have a longest dimension of at least about 0.25 μm, at least about 0.4 μm, at least about 0.5 μm or at least about 0.6 μm. In some embodiments, at least 50%, at least 55%, at least 60%, at least 65%, at least 70%, at least 75%, at least 80%, at least 85%, at least 90%, at least 95%, at least 97.5%, or at least 99%, per volume of the pigment microparticles, have a longest dimension within a range of 0.25 μm to 5 μm, within a range of 0.25 μm to 3 μm, within a range of 0.4 μm to 3 μm, or within a range of 0.4 μm to 1 μm. In some embodiments, these microparticles of pigments are additionally adapted to provide a UV-protective effect.

In particular embodiments, the pigment particles adapted to provide at least a UV-protective effect are pigment nanoparticles, wherein at least 50%, at least 55%, at least 60%, at least 65%, at least 70%, at least 75%, at least 80%, at least 85%, at least 90%, at least 95%, at least 97.5%, or at least 99%, per volume of the UV-protective agent nanoparticles, have a longest dimension of up to about 250 nm, up to about 200 nm, up to about 150 nm, up to about 100 nm, up to about 90 nm, up to about 80 nm, up to about 70 nm, or up to about 60 nm. In some embodiments, at least 10%, at least 15%, at least 20%, at least 25%, at least 30%, at least 35%, at least 40%, at least 45%, or at least 50%, per volume of the UV-protective agent nanoparticles, have a longest dimension of at least about 2 nm, at least about 5 nm, at least about 10 nm, at least about 15 nm, or at least about 20 nm. In some embodiments, the pigment nanoparticles have a D_{V}10 of 2 nm or more and a D_{V}99 of 250 nm or less, a D_{V}10 of 5 nm or more and a D_{V}99 of 200 nm or less, a D_{V}10 of 10 nm or more and a D_{V}99 of 150 nm or less, or a D_{V}10 of 15 nm or more and a D_{V}99 of 100 nm or less. In some embodiments, these nanoparticles of pigments are additionally adapted to provide a coloring effect.

In some embodiments, when the pigment particles are inorganic pigment particles, they can include metals and be, for instance, doped and undoped metal oxides, the metals being selected from a group comprising aluminum, barium, bismuth, cerium, cobalt, copper, iron, lanthanum, magnesium, manganese, nickel, titanium, vanadium, zinc, zirconium and combinations thereof.

In some embodiments, the pigment particles may further comprise a surface treatment, such coat or functionalization improving any desired feature, such as color stability, dispersibility, compatibility with other ingredients of the composition, and any like advantage as compared to a plain untreated pigment. By way of example, pigment particles may be surface treated to be rendered more hydrophobic/more compatible with the ingredients of the composition.

In particular embodiments, the inorganic pigment particles are regulatorily approved for use in contact with living subjects, though the present teachings prevent such direct contact, by embedding the pigment particles in polymer macroparticles. Example of inorganic pigments commonly used to provide at least a coloring effect include aluminum, bronze, copper and iron oxide, whereas inorganic pigments commonly used to provide at least a UV-protective effect include titanium dioxide and zinc oxide.

In some embodiments, the pigment particles (i.e. microparticles or nanoparticles regardless of their effect) are present in the swelled biodegradable polymer matrix macroparticles at a concentration from about 0.01 to about 400 wt. % (or w/w), from about 0.05 to about 300 wt. %, from about 0.1 to about 200 wt. %, from about 0.1 to about 100 wt. %, from about 0.1 to about 80 wt. %, from about 0.1 to about 60 wt. %, from about 0.1 to about 40 wt. %, from about 0.3 to about 30 wt. %, or from about 0.5 to about 25 wt. %, optionally at a concentration of about 1 wt. %, about 5 wt. %, about 10 wt. %, about 20 wt. %, about 50 wt. %, about 70 wt. %, about 100 wt. %, about 150 wt. %, or about 200 wt. % by weight of the thermoplastic biodegradable polymer.

In some embodiments, the pigment particles are present at a concentration from about 0.001 to about 80 wt. %, from about 0.001 to about 70 wt. %, from about 0.005 to about 60 wt. %, from about 0.005 to about 50 wt. %, from about 0.01 to about 40 wt. %, from about 0.1 to about 30 wt. %, from about 0.5 to about 20 wt. %, from about 0.7 to about 15 wt. %, or from about 0.7 to about 10 wt. % by weight of the swelled biodegradable polymer matrix macroparticles.

In some embodiments, the pigment particles are directly dispersed in the swollen polymer matrix during its milling, the pigments being optionally added as dry powders. In other embodiments, the pigment particles are separately dispersed in an oil carrier, whether by direct size reduction in the carrier or by redispersion of the pigment particles supplied or prepared in a medium other than the carrier. Such a step, to the extent necessary, may be referred to as a dispersing step. The dispersed particles can then be combined with the swollen polymer for their joint milling into macroparticles of swelled polymers including the pigment particles further dispersed therein.

In some embodiments, the oil carrier of the pigment particles is a non-aqueous carrier having a calculated HLB value between −10 and 25. Hence, the oil carrier of the pigment particles, if having a calculated HLB value of at least 3, may additionally be a non-aqueous polar carrier and serve as a swelling agent for the biodegradable thermoplastic polymer, or conversely the swelling agent of the biodegradable polymer can serve as an oil carrier for the pigment particles.

In some embodiments, the non-aqueous oil carrier is selected from a group consisting of saturated or unsaturated acid mono or diesters, fatty acid esters, cyclic organic esters, C₁₀-C₃₀ hydrocarbons, aromatic alcohols, glycols, polyols, fatty alcohols, aldehydes, ethers and combinations thereof.

In particular embodiments, the oil carrier serving as dispersing medium for the pigment particles is substantially non-volatile under the conditions (e.g., temperature and/or pressure) set for the dispersion. The non-aqueous oil carrier (which may optionally be polar and/or have a calculated HLB value of 3 or more) can be selected from a group of saturated or unsaturated acid mono or diesters, non-volatile fatty acid esters, fatty amines, aldehydes, ethers and aromatic alcohols, the group comprising 2-ethylhexyl lactate, acetyl tributyl citrate, acetyl triethyl hexyl citrate, acetyl triethyl citrate, benzyl alcohol, benzyl benzoate, butyl butyryl lactate, the mixture of caprylyl caprylate and caprylyl caprate, cinnamaldehyde (as can be found in cinnamon bark oil), dibutyl adipate, dibutyl maleate, dibutyl sebacate, diethyl succinate, diethylene glycol diethyl ether, dimethyl glutarate, dimethyl isosorbide, dimethyl maleate, dimethyl methylglutarate, gamma decalactone, glyceryl trioctanoate, lactic acid isoamyl ester, lauryl lactate, 1-menthyl lactate, menthalactone, N,N-bis-(2-hydroxyethyl)C₁₂-C₁₈-alkylamine octylamine, N,N-Dimethyl-dodecylamine, cetrimonium chloride, oleyl amine, octyl amine, n-pentyl benzoate, PEG-6 caprylic/capric glycerides, triacetin, triethyl citrate and and undecanoic lactone. A non-volatile liquid typically has a vapor pressure of 40 Pascal or less.

In some embodiments, the pigment particles, the swelling agents, the oil carriers of the pigment particles (if present), and the biodegradable polymer (or their respective and/or relative concentrations) are such, that the pigment particles can be dispersed in the macroparticles of swollen polymer in absence of any added dedicated dispersant, at least one of the foregoing constituents additionally acting as a dispersant. For illustration, the pigment particles and at least one of the swelling agent, oil carrier, if present, and biodegradable polymer includes functional groups that may serve to anchor the pigments particles, thereby stabilizing them in dispersed form once physically deagglomerated by adequate dispersing and/or milling. An exemplary oil carrier that possesses dispersing capabilities for pigment particles is C₁₂-C₁₈ alkyl benzoate.

Alternatively, in some embodiments, the composition further comprises a dispersant, adapted for dispersing the pigment particles either in presence of a biodegradable polymer being swelled by a swelling agent or in a separate liquid carrier, said dispersant being advantageously compatible with the biodegradable thermoplastic polymer, the pigment particles and the carrier being used. A dispersion is suitable if containing, as compared to a pre-dispersed state, a reduced amount of pigments aggregated or agglomerated as large secondary particles and an increased amount of pigments at their respective primary particle size or in relatively small clusters thereof, the relative proportions of each size (i.e. the particle size distribution) allowing the dispersed pigment particles to serve their intended purpose. Once dispersed with a dispersant, the pigment particles may be described as being associated (covalently or not) with the dispersant or as being “dispersant-laden”. Being dispersant-laden allows the pigment particles to be embedded within the biodegradable polymer matrix as individual particles or distinct small clusters thereof, the dispersant preventing or reducing their agglomeration after combining the pigment particles with the polymers (or even before, if the particles are preliminarily dispersed before being co-milled with the swollen polymer).

In some embodiments, the dispersant used for dispersing the particles in presence of either a non-aqueous polar swelling agent or a non-aqueous carrier is an oil-soluble dispersant optionally having a HLB value of no more than 9, or no more than 6, or no more than 3.

A dispersant suitable inter alia for pigment particles as herein disclosed can be selected in accordance with the surface properties of the pigments, including functional moieties thereon, if any. When the pigment particles are inorganic, the dispersant advantageously include an acidic moiety. In a particular embodiment, the dispersant includes or is derived from polyhydroxic stearic acid. In some embodiments, the swelling agent of the polymer and/or the oil carrier of the pigments is substantially non-volatile under the conditions (e.g., temperatures and/or pressure) they are respectively used (e.g., the liquid being non-volatile at a temperature of at least about 50° C.). However, this is not essential as the relative volatility of a liquid can be tolerated or even desired, if, for instance, facilitating its elimination from the composition under suitable conditions.

In some embodiments, the composition further comprises one or more of a second carrier, an excipient, an additive, and combinations thereof, each said compound being chemically compatible with the polymer, the pigment particles, the dispersant, and the other carriers being used. However, the second carrier is not required to swell the polymer. Carriers, excipients and additives that are cosmetically, dermatologically or pharmaceutically acceptable are preferred for use in living subjects, but such regulatory approvals may not be required for use of a composition according to the present teachings on the surfaces of inanimate objects.

Furthermore, though such excipients or additives are typically added to the composition following the preparation of the matrix elements comprising the pigment particles, and their optional transfer to a second carrier differing from the one having served as initial dispersing medium to the pigment particles and/or to the macroparticles of swollen polymer, this is not essential. Any such compound can be incorporated in any of the liquids or mixtures involved in the manufacturing process of the matrix elements (also referred to as matrix macroparticles). In such a case, the compound may need to be additionally compatible with the process and the step at which it may be introduced. For instance, a preserving agent, if added during the swelling of the thermoplastic biodegradable polymer, may have a greater heat resistance than a preserving agent added in a carrier due to be stored at a lower temperature.

As can be appreciated, if the matrix elements comprising the pigment particles are suspended in a liquid vehicle, corresponding for illustration to surplus of oil carrier and/or swelling agent not adsorbed by the swollen polymer, the relative concentration of pigment particles per total weight of the composition (assuming a fixed number of flakes) will decrease with an increase in the volume (and weight) of surrounding vehicle. If the vehicle is, or is replaced by, a relatively volatile liquid, a decrease in the volume (and weight) of the volatile vehicle as a result of evaporation will conversely lead to a relative increase in the concentration of the pigment particles per total composition. As further explained herein, the present teachings enable a substantial immobilization of the particles embedded in the swollen polymer matrix. Thus, while typical suspensions of particles cannot be concentrated above a certain limit without requiring amounts of dispersant that may become irritating and/or causing a collapse of the dispersion (e.g., particle aggregation/sedimentation etc.) annihilating their intended use, the present methods and compositions enable, if desired, use of relatively higher concentrations of particles without such deleterious effects.

In some embodiments, the composition comprises nanoparticles of an inorganic UV-protective agent (e.g., TiO₂ or ZnO, with or without surface treatment) dispersed as individual and distinct nanoparticles in macroparticles of a biodegradable polyester (e.g., PLA, PGLA, PCL, PHB, PHBV, PBS, PBSA, or blends thereof) entrapping (e.g., swollen by) a relatively polar oil having a polarity index of 25 mN/m or less, and/or a calculated HLB value of 3 or more. The relatively polar oil, which may represent one or more non-aqueous polar swelling agents alone or in combination with an additional oil carrier (which may have a calculated HLB of 3 or less), can be for instance at least one of a saturated or unsaturated acid mono or diester, such as butyl lactate, dimethyl glutarate, dimethyl maleate, dimethyl methyl glutarate, ethyl lactate and lactic acid isoamyl ester; a fatty acid ester, such as 2-ethylhexyl lactate, acetyl tributyl citrate, acetyl triethyl citrate, acetyl triethyl hexyl citrate, allyl hexanoate, benzyl benzoate, butyl butyryl lactate, a mixture of caprylyl caprate and caprylyl caprylate, dibutyl adipate, dibutyl maleate, dibutyl sebacate, diethyl succinate, glyceryl trioctanoate, 1-menthyl lactate, lauryl lactate, n-pentyl benzoate, PEG-6 caprylic/capric glycerides, triacetin and triethyl citrate; a cyclic organic ester, such as decanoic lactone, gamma decalactone, menthalactone and undecanoic lactone; a fatty amine, such as N,N-Bis-(2-hydroxyethyl)C₁₂-C₁₈-alkylamine octylamine, N,N-Dimethyl-dodecylamine, cetrimonium chloride, oleyl amine and octyl amine; an aromatic alcohol, such as benzyl alcohol; an aldehyde, such as cinnamaldehyde being the main component of natural cinnamon bark oil; and an ether, such as diethylene glycol diethyl ether and dimethyl isosorbide. Such oils, which can be extracted from the macroparticles of swollen polymer by any standard method (e.g., by leaching) can additionally be found in the composition as part of the medium in which the macroparticles are disposed. The composition may further comprise a polyhydroxic stearic acid (e.g., Pelemol® PHS-8, Dispersun OL100, or Dispersun OL300).

From the standpoint of a method for preparing corresponding compositions, it is emphasized that an oil carrier serving to initially disperse pigment particles need not be the same as a swelling agent serving to swell the biodegradable polymer. In such a case, the relatively hydrophilic oil (e.g., having polar groups such as esters, aldehydes, carboxyls and hydroxyls) found within the polymeric macroparticles might be a blend of the two liquids, in proportions additionally reflecting their relative elimination during the preparatory process. If one of the oils has a calculated HLB value of 3 or less, it can be inferred that it was used for dispersing the pigment particles prior to their embedment in the swollen polymer matrix.

In some embodiments, the pigment particles are dispersed and embedded in swelled biodegradable polymer matrix macroparticles shaped as flakes; wherein each flake of the swelled biodegradable polymer matrix flakes has a flake length (Lf), a flake width (Wf), and a flake thickness (Tf), the swelled biodegradable polymer matrix flakes having a dimensionless flake aspect ratio (Rf) defined by:

Rf=(Lf*Wf)/(Tf)²

wherein, with respect to a representative group containing at least 10 of the swelled biodegradable polymer matrix flakes, an average Rf is at least 5.

In some embodiments, the composition is formulated as a cosmetic composition being one of the following: (a) a skin-care composition for application to human or non-human animal skin; or (b) a hair-care composition for application to human or non-human animal hair.

In other embodiments, the composition can be formulated as a coating composition for application to an inanimate surface. For instance, the composition can be formulated as a lacquer, a varnish, a paint, or any such coating compositions.

According to a further aspect, there is provided a method of preparing a composition as described in any of the embodiments disclosed herein, the method comprising:
(a) swelling a thermoplastic biodegradable polymer having at least one of a melting temperature (Tm), a softening temperature (Ts) and a glass transition temperature (Tg) of 200° C. or less with at least one non-aqueous polar swelling agent having a calculated Hydrophilic-Lipophilic Balance (HLB) value of at least about 3; and
(b) milling the swelled polymer matrix in presence of a plurality of pigment particles, the milling being performed at at least one milling temperature for a milling period so as to size reduce and shape the matrix of swelled thermoplastic biodegradable polymer into macroparticles of swelled polymer matrix, whilst dispersing and embedding the pigment particles in the macroparticles, whereby substantially all pigment particles are embedded in the macroparticles of the swelled biodegradable polymer matrix.

The advantageous lack of free pigment particles non-embedded in the macroparticles of swelled thermoplastic biodegradable polymer matrix including a plurality of pigment particles dispersed therein prepared according to the present method can be verified by various techniques. For instance, the macroparticles may be separated from surplus liquid, or washed with surplus liquid, and the presence of pigment particles in the surplus liquid can be determined by DLS or LS. Alternatively, the embedment of substantially all pigment particles in the macroparticles of swelled polymer can be determined by microscope analysis, a ratio between a number of free non-embedded pigment particles and a number of macroparticles of swelled biodegradable polymer matrix being at most 10:1, at most 5:1, a most 2:1, at most 1:1, at most 1:2, at most 1:5, at most 1:10, or at most 1:20, as measured per unit area of a field of view of the microscope.

Generally, the swelling of step (a) is performed at at least one swelling temperature and/or under at least one swelling pressure for a swelling period so as to obtain a swelled polymer matrix including at least 5 wt. % of swelling agent per weight of polymer. The swelling step comprises combining the thermoplastic biodegradable polymer with the at least one swelling agent, this being optionally performed under ongoing agitation of said combination to provide a homogeneous paste of polymer matrix or morsels of a polymer matrix, the morsels being all similarly swelled, wherein the thermoplastic biodegradable polymer is at least partially swelled with the at least one swelling agent.

Step (b) comprises adding pigment particles to the swelled polymer matrix and milling the mixture of the pigment particles and swollen polymer, so as to size reduce the polymer matrix into swelled polymer matrix macroparticles, while incorporating and/or dispersing the pigment particles in the swelled biodegradable polymer matrix macroparticles. In some embodiments, the pigment particles are nanoparticles of a UV-protective agent.

The milling temperature at which the milling step is performed should be such that the swollen polymer and the swelling mixture are sufficiently relatively solid to be comminuted into smaller and smaller elements. Therefore, the milling temperature should be below the Tm or below the Ts of the swelled polymer (or of the swelling mixture if further affecting the thermal behavior of the swelled polymer). As milling at a relatively elevated temperature may facilitate kneading of the swelling mixture and dispersion and embedment of the pigment particles within the swollen polymer, the milling temperature can be relatively high and for instance be below one of a Tm and a Ts of the swelled polymer by only 5° C. However, this is not essential, and milling can also be performed at milling temperature inferior to one of a Tm and a Ts of the swelled polymer by 10° C. or more, 20° C. or more, 30° C. or more, or 40° C. or more. Preferably, the milling temperature should be higher than the Tg of the swollen polymer, to the extent the polymer is sufficiently crystalline to have a Tg, or should be above 20° C. Thus, in some embodiments, milling is performed at a temperature between 20° C. or a Tg of the swelled polymer and one of a Tm and a Ts of the swelled polymer less 5° C. (i.e., at most 195° C.). Furthermore, the milling temperature can be selected in accordance with the liquids that may be present in the swelling mixture (e.g., swelling agents and oil carriers), so that said liquids do not significantly evaporate during milling. However, this is not essential as volatility may be desired or volatile liquids replenished.

The pigment particles added to the milling of step (b) can be provided “as supplied” (e.g., as a dry powder or a suspension in a liquid), such supply corresponding to a desired particle size distribution and/or being in a medium compatible with the materials of the present compositions. Alternatively, the pigment particles can be preliminarily dispersed in an additional step. In such case, the plurality of pigment particles present during milling are pigment particles dispersed in a non-aqueous carrier being same or different than the one or more non-aqueous polar swelling agents swelling the polymer. In one embodiment, the dispersion step is performed for a dispersing period sufficient so that the dispersed pigment particles have a maximal particle size (D_{V}99) not exceeding 5 μm. This is however not essential as the milling of the pigment particles with the swollen polymer is expected to further reduce the size of the particles being concomitantly embedded.

While in the above brief description, the method of preparation of macroparticles of the present invention have been described for clarity as distinct steps, such steps need not be carried out separately or separated according to the above outlines, as shall be further detailed hereinbelow.

In some embodiments, the thermoplastic biodegradable polymer, swelling agent and oil carrier used in the preparation method of this aspect are substantially as described above and as further detailed herein with regards to the composition.

In a further aspect, there is provided a use for a composition as herein described, comprising afore-described macroparticles of swelled biodegradable polymer matrix comprising a thermoplastic biodegradable polymer swelled with at least one swelling agent (e.g., a relatively polar oil), and pigment particles, each of said pigment particles being optionally associated with a dispersant, wherein the pigment particles are dispersed and embedded in the swelled biodegradable polymer matrix macroparticles.

In some embodiments, the pigment particles are adapted to provide at least a coloring effect, in which case the composition can accordingly be used to provide a color to a surface upon which it is applied. If the composition is a cosmetic composition, containing one or more types of organic or inorganic pigments, the cosmetic composition can be used to provide a desirable color to skin, hair or nails of a subject.

In some embodiments, the pigment particles are additionally or alternatively adapted to provide at least a UV-protective effect, in which case the composition can accordingly be used to provide UV-protection against a harmful effect of UV radiation to a surface upon which it is applied. If the composition is a cosmetic composition, containing one or more types of inorganic nanoparticles of a UV-protective agent, the use of the composition is that of a sunscreen product for protecting skin or hair from UV radiation.

In some embodiments, when the compositions comprise UV-protective nanoparticles as pigment particles providing at least a UV-protective effect, these pigment particles may provide, alone or in combination, for broad UV absorption. A composition is said to have a UV-protective effect over a relatively broad UV range when the area under the curve (AUC) formed by the UV-absorption of the composition as a function of wavelength in the range of 280 nm to 400 nm (AUC₂₈₀₋₄₀₀) is at least 75%, at least 85% or at least 95% of the AUC formed by the same composition in the range of 280 nm to 700 nm (AUC₂₈₀₋₇₀₀).

In some embodiments, when the compositions comprise nanoparticles of a UV-protective agent providing alone or in combination a broad UV protection, the composition has a critical wavelength of at least 366 nm, such as 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, or greater than 392 nm.

While the UV-protective effect of a composition according to the present teachings is generally associated to the UV-absorbing capacity of pigment nanoparticles, such an effect can be alternatively, or additionally, achieved as a result of the presence of pigment microparticles capable of scattering UV-radiation. Hence, regardless of the specific size of the pigment particles dispersed and embedded in macroparticles of swollen biodegradable polymer, the present compositions can be UV-protective compositions if having at least one of:
i—a critical wavelength of at least 366 nm, at least 367 nm, at least 368 nm, at least 369 nm, or at least 370 nm; and
ii—an area under the curve (AUC) in a plot of absorption as a function of wavelength such that the AUC in the range of 280 nm to 400 nm (AUC₂₈₀₋₄₀₀) is at least 75%, at least 85% or at least 95% of the AUC range of 280 nm to 700 nm (AUC₂₈₀₋₇₀₀).

These and additional benefits and features of the disclosure will be better understood with reference to the following detailed description taken in conjunction with the figures and non-limiting examples.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the disclosure. For the sake of clarity, some objects depicted in the figures are not to scale.

In the Figures:

FIG. 1 is a line graph showing the Particle Size Distribution (PSD) of nanoparticles of titanium dioxide after dispersion with a dispersant in an oil carrier, expressed as volume percentage;

FIG. 2 is a line graph showing the PSD of macroparticles of swelled biodegradable polymer matrix after milling, expressed as volume percentage, the macroparticles of polymer incorporating the nanoparticles shown in FIG. 1;

FIG. 3A is a line graph showing the absorbance of neat titanium dioxide nanoparticles dispersed at various concentrations, prior to their incorporation into swelled biodegradable polymer matrix macroparticles of the present disclosure;

FIG. 3B is a line graph showing the absorbance of titanium dioxide nanoparticles incorporated at various concentrations into swelled biodegradable polymer matrix macroparticles of the present disclosure;

FIG. 4A is a line graph showing the absorbance of neat zinc oxide nanoparticles dispersed at various concentrations, prior to their incorporation into swelled biodegradable polymer matrix macroparticles of the present disclosure;

FIG. 4B is a line graph showing the absorbance of zinc oxide nanoparticles incorporated at various concentrations into swelled biodegradable polymer matrix macroparticles of the present disclosure;

FIG. 5A is a High-Resolution Scanning Electron Microscope (HR-SEM) image of a portion of a macroparticle of swelled biodegradable polymer matrix, with dispersed titanium dioxide nanoparticles;

FIG. 5B is a magnified view of the macroparticle shown in the HR-SEM image of FIG. 5A;

FIG. 6 is an HR-SEM image of a negative control showing macroparticles of swelled polymer wherein nanoparticles of titanium dioxide nanoparticles can be observed both embedded in the macroparticles and as free, non-embedded, pigments;

FIG. 7A schematically illustrates a cross sectional view on a surface of an example of conventionally prepared amorphous-shaped polymeric macroparticles containing pigment particles;

FIG. 7B schematically illustrates a cross sectional view on a surface of an example of conventionally prepared spherical-shaped polymeric macroparticles containing pigment particles;

FIG. 7C schematically illustrates in a perspective top view how the pigment particles entrapped in macroparticles as shown in FIGS. 7A and 7B could be distributed on the target surface, the contour of the polymer matrix being omitted for clarity;

FIG. 7D schematically illustrates in a cross-sectional view how pigment particles embedded in swelled polymer matrix macroparticles, such as flakes, may be applied to a target surface;

FIG. 7E schematically illustrates in a perspective top view how the pigment particles embedded and dispersed in swelled polymer matrix macroparticles as shown in FIG. 7D are distributed on the target surface, the contour of the polymer flakes being omitted for clarity;

FIG. 8A schematically illustrates a cross-sectional view of a swelled polymer matrix macroparticle having a tentacular flake shape;

FIG. 8B schematically illustrates a top view of a swelled polymer matrix macroparticle having a tentacular flake shape;

FIG. 9 provides a flow chart of one method for preparing compositions according to the present teachings; and

FIG. 10 is a line graph comparison of the absorbance, at a wavelength of 308 nm, between wet and dried compositions according to the present teachings containing various concentrations of titanium dioxide nanoparticles.

### DETAILED DESCRIPTION

The present disclosure provides compositions adapted to impart at least one of a UV-protective effect and a coloring effect. Uses of such compositions, in particular for cosmetic purposes, and methods of making them are also provided.

The compositions disclosed herein comprise macroparticles of swelled polymer matrix, the macroparticles comprising a thermoplastic biodegradable polymer swelled with at least one swelling agent, and a plurality of pigment particles, each of the pigment particles being associated with a dispersant, wherein the pigment particles are dispersed and embedded in the swelled biodegradable polymer matrix macroparticles.

A polymer is said to be biodegradable if breaking down relatively rapidly after fulfilling its purpose (e.g., by a bacterial decomposition process) to result in natural by-products. Biodegradable polymers are known, and new ones are being developed. However, despite its propensity to naturally decompose, it should be stable and durable enough for its intended use, during storage and application, which is particularly challenging if this use involves conditions that would enhance biodegradability. This is the case when a product forms a high surface area, as would be the case for coatings or cosmetics typically spread as relatively thin layers (e.g., on skin). This increases the exposure of the resulting film to factors (e.g., light, chemicals, or micro-organisms) promoting the degradation of the polymer.

Swellable polymers, as used herein, may have a relatively low degree of polymerization (to enable swelling), rendering the polymers of a macroparticle of swollen polymer more accessible to degradation initiators. For a polymer to be biodegradable, it is preferably hydrophilic, this easing interaction with water-soluble enzymes promoting polymer degradation. However, if used in cosmetic products, the polymer should preferably be hydrophobic, to avoid being washed away by sweat, humidity, rain, water-immersion and like conditions. As readily appreciated from the partial overview above of the challenges facing a developer of a pigmented composition, wherein the pigment particles are embedded in a polymer, finding a balance between these conflicting needs may render the task of selecting a biodegradable polymer suitable to swell in presence of a swelling agent, to embed pigment particles (and to preferably prevent their migration), all components of the composition (e.g., the polymers, the swelling agents, the pigments, their dispersants, etc.) being compatible with one another, quite daunting.

Fundamentally, a material or a chemical composition is compatible with another if it does not prevent its activity or does not reduce it to an extent that would significantly affect the intended purpose. Materials can be physically and/or chemically “compatible”. For instance, if applied on a living subject, the materials should be physically heat resistant at least to the body temperature of the subject, and advantageously to the temperature that may be perceived by the subject (e.g., if entering a sauna, sunbathing etc.) on the surface to which the material is applied. Materials should also be compatible with the methods used for the preparation of the composition. Materials can be physically compatible also if sharing for illustration a similar polarity, a similar calculated HLB value, or a similar index of refraction, the latter allowing the composition to be relatively clear, if so desired. This is not however essential, as colored compositions serving to conceal an underlying surface need not to be necessarily clear or transparent.

Similarly, from a chemical standpoint, a material should be compatible with all others it may interact with in the composition, for instance by sharing similar functional chemical groups or ones that may desirably interact with one another.

A contrario, and for illustration, a material would be incompatible if preventing the formation of a swollen matrix of biodegradable polymer capable of embedding the pigment particles. Such incompatibility can be readily tested and observed, for instance when a biodegradable polymer is unable to take up (be swollen by) a liquid, when the mixing of two liquids leads to phase separation, when a liquid (with or without a dispersant) does not succeed to properly wet the particles to be dispersed therein, and like inauspicious outcomes known to the skilled person.

While finding two materials compatible one with the other can be seen as a screening process, even if tedious, finding two materials additionally compatible with a third, or three materials additionally compatible with a fourth, and so on and so forth, may require more than extensive routine experimentation. As readily understood, this principle of compatibility, which can be affected not only by the chemical identity of the materials, but by their relative proportions according to the intended use, should preferably guide the selection of all materials necessary for the compositions disclosed herein.

The biodegradable polymers used in the present invention are swellable (advantageously, oil-swellable) thermoplastic homopolymers or copolymers, preferably clear, transparent and/or colorless for some implementations. Such a thermoplastic polymer, having any suitable crystallinity and/or isomeric composition, is said to be “swellable” if it can absorb and retain a swelling liquid, resulting in weight gain and/or a volume gain relative to its own mass or volume in its native form. Such a swelling liquid may also be considered to act as a plasticizer, rendering the swollen polymer softer and more malleable, facilitating for illustration the dispersion of the pigment particles therein and the pulverization of the matrix enabling the size reduction of morsels of the swollen matrix and the shaping of the macroparticles. The swelling agent may also decrease the melting, softening or glass transition temperature of the biodegradable polymer, once swollen therewith, thus lowering the temperatures at which the swollen polymer matrix can be prepared and the macroparticles of the composition shaped. Hence, a “swelled biodegradable polymer” can alternatively be considered as a “plasticized biodegradable polymer”.

Biodegradable polymers, suitable for the purpose of the present invention have at least one of a softening temperature (Ts), a melting temperature (Tm), and a glass transition temperature (Tg) not exceeding of 200° C. In some embodiments, the biodegradable swellable thermoplastic polymers have at least one of a softening temperature (Ts) and a melting temperature (Tm), of at least 20° C., at least 30° C., at least 40° C., at least 50° C., or at least 60° C. In some embodiments, at least one of the softening temperature (Ts) and the melting temperature (Tm) of the biodegradable swellable thermoplastic polymer is within a range of 20° C. to 200° C., within a range of 30° C. to 190° C., or within a range of 40° C. to 180° C. A polymer fulfilling at least one of said thermal properties is expected to contribute to the ability of a composition prepared therefrom to not excessively soften when applied to a surface at a temperature range in which the composition would normally be used (e.g., would not melt at body temperature of about 37° C. if applied to a human subject).

The degree of swelling at saturation typically indicates the density between polymer chains, wherein softer “low-density” polymers generally have a higher absorbent capacity, allowing them to swell to a larger degree, than harder “high-density” polymers. Biodegradable thermoplastic polymers can also be defined by their rate of crystallinity, where crystallinity is the indication of amount of crystalline region in the polymer with respect to amorphous content. Generally, more amorphous biodegradable polymers (e.g., having a relatively low amount of crystalline portion, such as 30% or less) may swell more than semi-crystalline or crystalline polymers.

A thermoplastic biodegradable polymer showing a weight gain and/or a volume gain of at least 5%, at least 10%, at least 15%, at least 20% when immersed in an excess of swelling agent for a period of up to 4 days, at a temperature of about 50° C. under standard pressure of approximately 101.325 kiloPascal (kPa) is deemed suitably “swellable” by the swelling agent. Advantageously, a swellable polymer once suitably swelled can be morselized under low shear, such phenomenon being deemed predictive of its ability to be shaped into macroparticles and to embed dispersed pigment particles.

While thermoplastic biodegradable polymers that are swellable, as above-described, can be preferred for the preparation of matrix macroparticles, the biodegradable polymer needs not necessarily be swelled to its greatest possible extent (i.e., saturation) to be used according to the present teachings. As used herein, the term “swelled” with regard to a biodegradable polymer, refers to a swollen polymer matrix, which shows a weight gain of at least 5% under elected swelling conditions, as compared to the polymer weight prior to said swelling. It is believed that the degree of actual swelling may facilitate the size-reduction of the polymer matrix into matrix elements. For this purpose, the thermoplastic biodegradable polymer needs to be sufficiently swollen (i.e., soften) to permit kneading of the polymer into individual elements, but not too soft so that the resulting elements would lose shape (e.g., flow and merge into neighbouring elements). Proper swelling may also facilitate the later penetration of added pigment particles and their dispersion during the co-milling with the swollen matrix, while ensuring the relative immobilization of the solid pigment particles within the swollen matrix elements, the amount of swelling agent actually absorbed and/or retained during this process possibly additionally depending upon the manufacturing conditions.

It is reminded in this context that while a swelling of at least 5% may be favorable during the manufacturing of the composition, a macroparticle may include a lesser amount of swelling agent in a final composition intended for use. For illustration, a relatively volatile liquid may be used, the macroparticles may be dried, or their contents otherwise leached out, following the end of the process.

While in the method that can be used to determine the swellability of a polymer by a particular liquid specific conditions have been set, the actual swelling of the swellable biodegradable polymer with the desired swelling agent(s) can be performed under a variety of swelling conditions, elevated temperatures (i.e. above 30° C., e.g., at 40° C. or more, at 50° C. or more, at 60° C. or more, at 75° C. or more, or at 90° C. or more) and/or elevated pressure (i.e. above about 100 kPa, e.g., at 125 kPa or more, 150 kPa or more, 175 kPa or more, 200 kPa or more, 250 kPa or more, or 300 kPa or more) typically accelerating the swelling process (i.e., shortening the duration of the swelling period). Conversely, swelling the swellable biodegradable polymer with a swelling agent under conditions less favorable than arbitrarily set to assess the swellability of a polymer by a specific agent, such as at a temperature of less than 50° C. and/or a reduced pressure of less than 100 kPa, may prolong the swelling process, if desired.

Mixing of the swellable biodegradable polymer with or within the swelling agent can also shorten the swelling period, a regular agitation additionally ensuring that all morsels of swelling polymer matrix swell in a relatively uniform manner, the swelled matrix whether forming a continuous mass or separate morsels behaving reasonably homogeneously with respect to subsequent steps of the method and results expected therefrom. If excess of swelling agent is used during swelling, it can be optionally removed before proceeding to following step(s).

The temperature at which swelling is performed is typically selected according to temperatures characterizing the substances involved in the process, for instance by taking into account a Ts, Tm and/or Tg characterizing the swellable biodegradable thermoplastic polymer. Such characterizing temperatures are typically determined by thermogravimetric analysis at standard pressure of 1 atmosphere, but one can readily appreciate that a change in the properties of the substance reflected by these temperatures can alternatively take place at a lower temperature or a higher temperature, if the pressure in a sealed chamber hosting the process were to be accordingly reduced or increased. Therefore, while in the description of a method suitable for the preparation of a composition according to the present teachings, reference can be made to specific temperatures and duration of times assuming the process is carried out under standard pressure, such guidance should not be viewed as limiting and all temperatures and durations achieving a similar outcome with respect to the behavior of the swelled polymer matrix are encompassed. It is noted in this context that while the Tm and/or Tg of the polymer may set relatively clear temperatures below and above which a polymer may display a distinct behavior, this typically does not apply to the Ts. In view of their viscoelastic properties a polymer or a swelled polymer may remain “sufficiently solid” even at a temperature moderately higher than its formal softening point.

The duration of swelling will inter alia depend on the biodegradable polymer being swelled, the swelling agent(s) being used, the swelling conditions (e.g., temperature, pressure, and/or agitation), and the desired extent of swelling. The swelling period can be of at least 30 minutes and at most 4 days.

As mentioned, a “suitable softening”/swelling of a biodegradable polymer needs to balance between yielding a polymer mass malleable enough (i.e., sufficiently soft) to incorporate therein the pigment particles, the macroparticles formed therefrom being on the other hand sufficiently “tough” to retain their intended shapes and fulfil their intended roles. For this purpose, in some embodiments, the swelled thermoplastic biodegradable polymer has at least one of a Tm and a Ts being above 20° C. In some preferred embodiments, the swollen polymer matrix has at least one of said temperatures (Tm or Ts) being 30° C. or more, 40° C. or more, 50° C. or more, or 60° C. or more. As the temperatures characterizing a swelled polymer are typically lower than their corresponding temperatures in the unswelled polymer, all may be less than 200° C., less than 190° C., less than 180° C., less than 170° C., less than 160° C., or less than 150° C.

If a combination of biodegradable polymers is used and/or if the mixture of biodegradable polymer(s), swelling agent(s) and pigment particles (hereinafter the “swelling mixture”) further comprises ingredients that may impact such softening property of the resulting matrix elements (e.g., rheological modifiers, dispersants, preservatives, or any like material which may have a plasticizing effect), then additionally and alternatively, the aforesaid thermal behavior would apply to the entire swelling mixture. Hence, in some embodiments, the swelled thermoplastic biodegradable polymer and/or the swelling mixture have at least one of a Tm and a Ts being in a range of 20° C. to 200° C., 30° C. to 190° C., 40° C. to 180° C., 50° C. to 170° C., 50° C. to 160° C., or 50° C. to 150° C.

Such thermal behavior and characterizing temperatures can be assessed while preparing the swollen polymer matrix (or swelling mixture) or upon completion of the preparation method of the composition. Such features (the macroparticles displaying at least one of a Tm and a Ts being greater than 20° C. and so on) can additionally reduce the stickiness of macroparticles amongst themselves, if so desired.

Biodegradable polymers suitable for the present compositions contain hydrolysable functional groups, and they can be natural or synthetic polymers. Synthetic biodegradable polymers are selected from polyesters, polyamides, polyurethanes, polyureas, poly(amide-enamine)s, polyanhydrides, enantiomers thereof, copolymers thereof and combinations thereof. Such polymers may be identified according to their respective characteristic functional groups as detectable by standard methods known to the skilled persons, for instance, by Fourier-transform infrared (FTIR) spectroscopy. As these polymers are thermoplastic, heating of the macroparticles may soften them sufficiently so as to reshape them to adopt a new configuration upon cooling.

To the extent that the monomers forming the biodegradable polymers have chiral centers, all enantiomers and stereoisomers are encompassed. For illustration, lactic acid (2-hydroxypropionic acid, LA), exists as two enantiomers, L- and D-lactic acid, so that PLA has stereoisomers, such as poly(L-lactide) (PLLA), poly(D-lactide) (PDLA), and poly(DL-lactide) (PDLLA). A biodegradable polymer may therefore be a mixture of isomers of a same molecule or a specific stereoisomer (or a stereo copolymer).

Additionally, a biodegradable polymer can be a blend of different suitable thermoplastic molecules (e.g., PLA and PHB which can be swelled with a similar agent) or a copolymer including at least one of the aforementioned thermoplastic biodegradable polymer, such copolymers may contribute to the biocompatibility, biodegradability and mechanical and optical properties. In some embodiments, the biodegradable thermoplastic copolymer is selected from: poly(lactic-co-glycolic acid) (PLGA), poly(3-hydroxybutyrate-co-3-hydroxy-valerate) (PHBV), poly(butylene succinate-co-adipate) (PBSA), poly[oligo(tetra-methylene succinate)-co(tetramethylene carbonate), poly(butylene adipate-co-terephtalate) (PBAT).

Biodegradable polyesters can be selected from: poly(lactic acid) (PLA), poly(lactide-co-glycolide) (PLGA), polycaprolactone (PCL), poly(glycolic acid) (PGA), polyhydroxyalkanoate, such as polyhydroxybutyrate (PHB) and poly(3-hydroxybutyrate-co-3-hydroxyvalerate) (PHBV); poly(alkene dicarboxylate), such as poly(butylene succinate) (PBS), poly(butylene succinate-co-adipate) (PBSA) and poly(ethylene succinate) (PES); polycarbonate such as poly(trimethylene carbonate) (PTMC), poly(propylene carbonate) (PPC) and poly[oligo(tetramethylene succinate)-co(tetramethylene carbonate); poly(p-dioxanone) (PPDO); aliphatic-aromatic copolyesters, such as poly(ethylene terephtalate), poly(butylene adipate-co-terephtalate) (PBAT).

Biodegradable polyurethanes can be synthesized from diisocyanate, a chain extender and a polyol, for example, toluene-2,4-diisocyanate combined with dihydroxyl terminated PLLA (L-isomer of PLA) and PBS, or from poly(F-caprolactone) and 4,4-diphenylmethane diisocyanate, extended with chitin and 1,4-butane diol. Lysine diisocyanate or 1,4-diiso-cyanatobutane can be combined with polyester diols based on lactide or F-caprolactone, to yield the biodegradable polyurethane.

While some of the afore-said polymers have natural counterparts, such materials are typically commercially available in artificially prepared form, so that the entire group is often considered as representative of synthetic polymers.

Biodegradable polymers of natural origin can be selected from polysaccharides, such as thermoplastic starch (TPS), cellulose chitin, chitosan, fibers, gums and pullulan; polypeptides, such as corn zein, wheat gluten, soy protein, collagen, gelatin, casein, caseinates and whey proteins; and derivatives thereof. Shellac, a bioadhesive resin, is another suitable biodegradable polymers of natural origin, preferably used unwaxed and bleached. In this context, a derivative relates to a chemically modified polymer, containing at least one substituent, not present in the unmodified polymer, e.g., a polymer which has been chemically modified.

Some swellable biodegradable polymers can be synthesized using naturally occurring monomers, including by way of example polyesters such as PLA, PHB and PHBV.

In a particular embodiment, the swellable biodegradable polymer is PLA. In another particular embodiment, the swellable biodegradable polymer is PLGA. In yet another embodiment, the biodegradable polymer is PHB. In yet a further embodiment, the biodegradable polymer is PHBV. In another embodiment, the biodegradable polymer is PBS. In yet another embodiment, the biodegradable polymer is PBSA. In a further embodiment, the biodegradable polymer is a mixture of PLA and PHB. In a further embodiment, the biodegradable polymer is a mixture of PLA and PCL.

In some embodiments, the swellable biodegradable polymer is present in the swelled biodegradable polymer matrix macroparticles at a concentration from about 3 to about 90 wt. %, from about 3 to about 85 wt. %, from about 3 to about 75 wt. %, from about 3 to about 75 wt. %, from about 3 to about 60 wt. %, from about 3 to about 50 wt. %, from about 5 to about 40 wt. %, from about 7 to about 35 wt. %, or from about 10 to about 30 wt. % by weight of the macroparticles, optionally at a concentration of about 5 wt. %, about 10 wt. %, about 15 wt. %, about 20 wt. %, about 30 wt. %, or about 35 wt. % by weight of the swelled biodegradable polymer matrix macroparticles.

In some embodiments, the swelled biodegradable polymeric matrix prevents, reduces, or delays the migration of pigment particles out of the matrix macroparticles, resulting in low amounts or substantially null amounts of free, non-embedded pigments in the composition. This substantial immobilization of the pigment particles, or lack of migration, should be maintained during storage of the composition and at least as long as the composition is applied to or contacting a living subject. Such free pigments can be undesired in, e.g., cosmetic compositions, where direct contact of the pigments with the skin could be hazardous, in particular when the pigments are nanoparticles that may permeate the skin barrier. In some embodiments, the composition is substantially devoid of free pigment particles non-embedded in the macroparticles of the swelled biodegradable polymer matrix, the ratio between a number of free non-embedded pigment particles and a number of macroparticles of swelled biodegradable polymer matrix (containing pigment particles embedded therein) is at most 10:1, at most 5:1, a most 2:1, at most 1:1, at most 1:2, at most 1:5, at most 1:10, or at most 1:20. This ratio can be readily ascertained by microscopic analysis (e.g., by SEM or HR-SEM) of a sample of the composition over a predetermined unit area, e.g., of 10 μm by 10 μm, counting the number of free pigment particles, if any, as compared to the number of macroparticles in the same field of view, this analysis being repeated (e.g., at least three times) to provide sufficient statistical significance. A composition in which the ratio of free pigments to polymeric macroparticles (as can be measured per unit area) is as afore-set can be considered substantially devoid of free pigment particles, which are not embedded in the macroparticles. But other methods may be used to assess this matter.

For instance, the presence or absence of free pigment particles can also be ascertained by separating the macroparticles from the composition (e.g., by centrifugation and isolation of the fractions) and analysing the amount of pigment particles, e.g., by microscopic or spectrophotometric methods, at least in the liquid fraction.

In some embodiments, the thermoplastic biodegradable polymer is swelled with at least one swelling agent, comprising or consisting of a non-aqueous polar oil.

Oils are generally defined as substances substantially water immiscible at room temperature (circa 23° C.) and atmospheric pressure, and typically, but not necessarily, liquid under such conditions. They are generally non-aqueous, non-polar, provide a greasy-feel and can be characterized, among other things, by the source of the oil, the degree of saturation/unsaturation, the type of fatty acids and/or their relative content, the length of carbon chains, and the like typical parameters. The afore-mentioned chemical characteristics may affect physical behaviour, for instance the melting point and/or the boiling point and/or the viscosity and/or volatility of the oil, or mixture thereof, at temperatures of interest (e.g., to the formulation process during dispersing, milling or matrix preparation, to the application process, to the intended use, etc.). The oil may additionally affect the melting point and/or the softening point of the thermoplastic biodegradable polymers or blends thereof upon a combination therewith in a polymeric matrix.

As used herein, the term “oil” is not intended to be limited only to substances formally referred to as such, and an “oil carrier” or an “oily” swelling agent or an “oil-compatible” material, and all such terms relating to oils as used herein with reference to the present compositions or methods, further encompass slightly viscous liquids (e.g., that have a fluidity similar to conventional oils) but may be relatively polar and/or partially miscible in water and/or non-greasy. Taking for illustration a swelling agent adapted to swell the biodegradable swellable thermoplastic polymers according to the present teachings, it may in fact be a relatively polar oily substance, while an oil carrier that may serve to disperse pigment particles (if required) could additionally be relatively non-polar.

The relative polarity of a material can be determined by experimentation or calculated (e.g., its HLB value). In the Davies method, the computation of the HLB value is based upon different values being assigned to hydrophilic or lipophilic structural groups in a molecule, exemplary values for common groups being available inter alia in Guo W. et al., Journal of Colloid and Interface Science 298 (2006) 441-450.

The HLB value of the molecule can then be estimated by the following equation, based on the various groups forming the molecule: HLB=7+Σ(hydrophilic group values)+Σ(lipophilic group values). For illustration, a hydrocarbon, such as an isoparaffin oil having a chain length of fifteen carbon atoms may have a negative calculated HLB value of −0.125, being non-polar, whereas benzyl alcohol, which is a fatty aromatic alcohol can be considered a relatively polar oil with a calculated HLB value of almost 7.

In some embodiments, liquids referred to as oils, constituting at least part of a swelling agent (capable of swelling an oil-swellable biodegradable polymer) or being the swelling agent, have an HLB value, as calculated by the method of Davies, of 3 or more, 4 or more, 5 or more, or 6 or more. Such oils (e.g., swelling agents, oil carriers, and blends thereof) can be considered relatively polar (or relatively more polar than conventionally non-polar oils which would optionally conversely display a calculated HLB value of less than 3). In some embodiments, the swelling agents have a calculated HLB value of 25 or less, 20 or less, 15 or less. In some embodiments, the swelling agents have a calculated HLB value within a range of 3 to 25, within a range of 3 to 20, within a range of 4 to 20, or within a range of 5 to 15.

Some oils may act as partial plasticizers with respect to some biodegradable polymers. In such a case, a first oil serving as swelling agent may be used to decrease the softening point of the combined swelling mixture to a temperature facilitating the milling of the swollen biodegradable polymer with the pigment particles under conditions (e.g., temperature and/or pressure) at which the mixture being milled is below such softening turning point, while being adapted to the softening behavior tolerable for each intended product.

In some embodiments, the pigment particles can be added to the biodegradable thermoplastic polymer as a dry powder, the mixing of the polymer with its swelling agent(s) and/or the milling of the swelled polymer additionally serving to disperse the pigment particles. In some embodiments, the pigment particles can be dispersed in a second oil (which can also be referred to as a carrier or an oil carrier) prior to being added to the (optionally pre-swollen) biodegradable polymer matrix, the second oil being in some embodiments capable of decreasing the softening point of the milling mixture (or further decreasing it in the event the first oil displayed such a plasticizing effect). In some embodiments, the first (swelling) oil and the second (pigment carrier) oil are the same. In other embodiments, the first oil and the second oil are different.

As mentioned, once the matrix macroparticles are obtained, the first oil adapted to swell the biodegradable polymer or the second oil adapted to serve as medium for the dispersion of the pigments, to the extent used in the preparation, can be at least partially replaced by a third liquid. As the macroparticles of swollen biodegradable polymer comprising the dispersed pigment particles embedded therein are typically hydrophobic, the third liquid can be a third oil. In contrast with the first and second oil which must or may swell the polymer, and may lower the softening point of the biodegradable polymer, the third oil can be inactive in these respects. Namely, the third oil being added at a later stage, it does not need to swell the biodegradable polymer matrix elements nor modify their characterizing temperatures, though it may be selected to do so if required. The third oil may only be serving as a vehicle to such macroparticles now including the pigment particles, optionally with residual amount of the first oil and second oil, if any. The third liquid need not be an oil and any other material adapted for the transient processing or the final formulation of the macroparticles into a desired end-product may be used.

For illustration, if isolating the macroparticles from any surrounding media is desired, they can be decanted, centrifuged, filtered and rinsed with any suitably third liquid, such liquid being preferably volatile to facilitate its elimination (e.g., being a volatile oil, a short alcohol or a short ketone).

Biodegradable polymers having too high a softening point or melting point are believed to be less suitable as they would tend to be non swellable. Typically, suitable thermoplastic biodegradable polymers have at least one of a softening point and a melting point not exceeding 200° C., or possibly not greater than 180° C. Polymers (such as the biodegradable polymers of the present invention) having a higher degree of crystallinity will have a melting point, whereas more amorphous polymers are more likely to be characterized by a softening point.

In some embodiments, the swelling agent and the oil carrier can be independently selected from the group consisting of a mineral oil, a natural oil, a vegetal oil, an essential oil, a synthetic oil and combinations thereof. In some embodiments, the swelling agent and/or oil carrier has a polarity index of 25 mN/m or less, 20 mN/m or less or 15 mN/m or less.

Any combinations of oils can be suitable as long as they form a homogeneous fully miscible mixture having the desired relative polarity (as can be estimated from their respective calculated HLB value and/or polarity index, or from the mathematical average of such values according to their respective presence in the mixture). The oil mix must be compatible with the use envisaged (from polymer swelling to application to target surface, through the incorporation of the pigment particles and the milling forming the swelled biodegradable polymer matrix macroparticles) and such compatibility can be determined by routine experimentation as detailed for the individual oils. For illustration, an oil or a blend thereof will be sufficiently polar to swell a particular biodegradable polymer if fulfilling the minimal conditions set for swellability of a polymer by a swelling agent (i.e., a weight gain of at least 5% at 50° C. in no more than 4 days). An oil (alone or blended with others) will be compatible with the pigment particles if being capable of wetting them. In some embodiments, the oil is a cosmetically acceptable oil conventionally used in the preparation of personal care products.

Non-limiting examples of synthetic oils, being suitable as swelling agents and relatively polar oil carriers (e.g., having a calculated HLB value of at least 3, or experimentally demonstrating the swellability of the polymer), include esters, such as saturated or unsaturated acid mono or diesters (e.g., butyl lactate, dimethyl glutarate, dimethyl maleate, dimethyl methyl glutarate, ethyl lactate and lactic acid isoamyl ester), fatty acid esters (e.g., 2-ethylhexyl lactate, acetyl tributyl citrate, acetyl triethyl citrate, acetyl triethyl hexyl citrate, allyl hexanoate, butyl butyryl lactate, dibutyl adipate, dibutyl maleate, dibutyl sebacate, diethyl succinate, 1-menthyl lactate, lauryl lactate, PEG-6 caprylic/capric glycerides (as commercially available, for instance, from BASF as Cetiol® 767), triacetin and triethyl citrate) and cyclic organic esters (e.g., decanoic lactone, gamma butyrolactone, gamma decalactone, menthalactone, octanoic lactone, nonanoic lactone, propylene carbonate and undecanoic lactone); fatty amines (e.g., N,N-bis-(2-hydroxyethyl)C₁₂-C₁₈-alkylamine octylamine, N,N-dimethyl-dodecylamine, cetrimonium chloride, oleyl amine and octyl amine); aromatic alcohols (e.g., 1-phenoxy-2-propanol, 1-phenyl ethanol, 2-phenyl ethanol and benzyl alcohol); glycols (e.g., propylene glycol, polyethylene glycols and hexylene glycol); polyols (e.g., glycerol); aldehydes (e.g., cinnamaldehyde, cuminaldehyde and citral); and ethers (e.g., diethylene glycol diethyl ether, dimethyl isosorbide and diphenyl ether).

Non-limiting examples of suitable vegetal oils include argan oil, chokeberry (seed) oil, avocado oil, apricot kernel oil, peach (pits) oil, canola oil, nigella oil, pumpkin seed oil, wild rose (seeds) oil, pomegranate seeds oil, jojoba oil, cocoa butter, wheat sprout oil, coconut oil, safflower oil, corn oil, camelina oil, flax seed oil, macadamia oil, raspberries seeds oil, meadowfoam seeds oil, passiflora seeds oil, almond oil, neem oil, moringa oil, borago oil, olive oil, peanuts oil, hazelnuts oil, walnut oil, palm oil, papaya seeds oil, parsley seeds oil, seabuckthorn oil, castor oil, rice oil, sesame oil, shea butter, sunflower oil, soybean oil, tamanu oil, evening primrose oil, grape seeds oil, cranberry seeds oil. Vegetal oils being relatively polar (e.g., having a calculated HLB of 3 or more, or experimentally demonstrating the swellability of the polymer) can be used, alone or in combination with other oils, as swelling agents and oil carriers. Vegetal oils being relatively non-polar (e.g., having a calculated HLB of less than 3) can be used, alone or in combination with other oils, as oil carriers.

Non-limiting examples of essential oils include agar oil, ajwain oil, angelica root oil, anise oil, asafetida, balsam of Peru, basil oil, bay oil, bergamot oil, black pepper oil, buchu oil, birch oil, camphor, cannabis flower essential oil, caraway oil, cardamom seed oil, carrot seed oil, cashew oil, cedarwood oil, chamomile oil, calamus root oil, cinnamon oil, cinnamon bark oil, cistus oil, citron oil, citronella oil, clary sage oil, clove leaf oil, coffee oil, coriander seed oil, costmary oil, costus root oil, cranberry seed oil, cubeb oil, cumin oil, cypress oil, curry leaf oil, davana oil, dill oil, elecampane oil, eucalyptus oil, fennel seed oil, fenugreek oil, fir oil, frankincense oil, galangal oil, galbanum oil, ginger oil, goldenrod oil, grapefruit oil, henna oil, helichrysum oil, hickory nut oil, horseradish oil, hyssop oil, Idaho tansy oil, jasmine oil, juniper berry oil, Laurus nobilis oil, lavender oil, ledum oil, lemon oil, lemongrass oil, lime oil, litsea cubeba oil, linaloe oil, mandarin oil, marjoram oil, melissa oil, mentha arvenis oil, moringa oil, mountain savory oil, mugwort oil, mustard oil, myrrh oil, myrtle oil, neem oil, neroli oil, nutmeg oil, orange oil, oregano oil, orris oil, palo santo oil, parsley oil, patchouli oil, perilla oil, pennyroyal oil, peppermint oil, petitgrain oil, pine oil, ravensara oil, red cedar oil, Roman chamomile oil, rose oil, rosehip oil, rosemary oil, rosewood oil, sage oil, sandalwood oil, sassafras oil, savory oil, schisandra oil, spearmint oil, spikenard oil, spruce oil, star anise oil, tangerine oil, tarragon oil, tea tree oil, thyme oil, tsuga oil, turmeric oil, valerian oil, vetiver oil, western red cedar oil, wintergreen oil, yarrow oil, ylang-yland oil and zedoary oil. Essential oils being relatively polar (e.g., having a calculated HLB of 3 or more, or experimentally demonstrating the swellability of the polymer) can be used, alone or in combination with other oils, as swelling agents and oil carriers. Essential oils being relatively non-polar (e.g., having a calculated HLB of less than 3) can be used, alone or in combination with other oils, as oil carriers.

In some embodiments, the swelling agent is selected from: a saturated or unsaturated acid mono or diester; a fatty acid ester; a cyclic organic ester; an aromatic alcohol; an ether; and an essential oil. In particular embodiments, the swelling agent is selected from: 2-ethylhexyl lactate, acetyl tributyl citrate, acetyl triethyl hexyl citrate, acetyl triethyl citrate, allyl hexanoate, benzyl alcohol, benzyl benzoate, butyl butyryllactate, butyl lactate, cinnamon bark oil, dibutyl adipate, dibutyl maleate, dibutyl sebacate, diethyl succinate, diethylene glycol diethyl ether, dimethyl glutarate, dimethyl isosorbide, dimethyl maleate, dimethyl methyl glutarate, ethyl lactate, gamma decalactone, lactic acid isoamyl ester, lauryl lactate, 1-menthyl lactate, menthalactone, n-pentyl benzoate, PEG-6 caprylic/capric glycerides, triacetin, triethyl citrate and undecanoic lactone.

In some embodiments, the weight per weight ratio of swelling agent to the biodegradable polymer is between 0.2:1 and 20:1, between 0.2:1 and 15:1, between 0.2:1 and 10:1, between 0.2:1 and 5:1, between 0.7:1 and 19:1, between 1:1 and 17:1, between 1.5:1 and 16:1, between 2:1 and 14:1, between 2:1 and 10:1, or between 2:1 and 8:1.

In some embodiments, the swelling agent is present in the macroparticles of swelled biodegradable polymer matrix containing the pigment particles at a concentration from about 5 to about 50 wt. %, from about 10 to about 35 wt. %, from about 50 to about 80 wt. %, from about 55 to about 80 wt. %, or from about 60 to about 80 wt. % by weight of the macroparticles, optionally at a concentration of about 60 wt. %, about 65 wt. %, about 70 wt. %, about 75 wt. %, or about 80 wt. % by weight of the swelled biodegradable polymer matrix macroparticles.

As mentioned, a portion of the volume of the swelling agent may have been contributed by the carrier in which the pigments particles are supplied or dispersed prior to incorporation in the polymer matrix, even if said carrier is not by itself swelling.

In some embodiments, the biodegradable polymer and swelling agents can be selected so that their refractive indices values are within a range of at most 10% from one another, their combination thus creating a relatively clear polymeric matrix. Such clear biodegradable polymeric may be desirable if colored pigment microparticles later embedded into the matrix are to tint an underlying surface, without necessarily concealing it. When the pigment particles are UV-protective agent nanoparticles, their embedment in clear macroparticles of biodegradable polymers would render the composition transparent and substantially invisible to the naked eye. Relatively similar refractive indices are however not essential, for instance when the composition can be opaquer, in particular if the composition is intended to cover and hide the color of the surface upon which it is applied.

The fact that polymeric macroparticles are swelled may be determined by thermogravimetric analysis (TGA), wherein upon heating of a sample of macroparticles isolated from the composition, a weight loss is observed, supposedly as the result of the elimination of the swelling agent from the polymeric matrix.

Moreover, when the thermoplastic biodegradable polymer in a composition is known or identified, a Differential Scanning Calorimetry (DSC) analysis can be conducted to estimate whether it was swelled by a swelling agent. Basically, a Tm or Ts of the polymeric macroparticles, isolated from the composition, can be determined by DSC analysis, and the obtained values can be compared to the known Tm or Ts of the known polymer. If the measured values are lower than the known corresponding temperatures of the polymer in its natural, un-swelled form, this may indicate that the polymer present in the macroparticles has been swelled.

Having established, or assuming, that the macroparticles are of a swelled thermoplastic biodegradable polymer, the identity of the swelling agent may be determined by extracting the liquids entrapped within the polymer matrix and submitting them to chemical analysis. For instance, the polymeric macroparticles can undergo a leaching process, whereby the swelling agent is extracted out of the polymeric matrix (e.g., by centrifugation, pressure decantation, or heating), and may be further analysed by known methods, such as FTIR and liquid mass spectrometry (LMS).

The thermoplastic biodegradable polymeric matrix further contains pigment particles, embedded therein. Such pigment particles can assume a variety of shapes, such as sphere-like, rod-like or platelet like. Regardless of their shapes, the pigment particles as used according to the present teachings, can be classified by their characterizing size. They can be nanoparticles, in particular when they are adapted to provide at least a UV-protective effect. Alternatively, the pigment particles can be microparticles, especially when adapted to provide at least a coloring effect. As explained, these effects are not mutually exclusive but only intended to reflect the predominant effect expected in each size range, the only major distinction between the two optionally being in the transparency or opacity that can be obtained. All pigment particles can provide a UV-protective effect by absorbing or scattering the UV radiation.

As used herein, the term “nanoparticles” refers to particles, wherein the size of a longest dimension is about 250 nm or less, about 200 nm or less, about 150 nm or less, about 100 nm or less, about 90 nm or less, about 80 nm or less, about 70 nm or less, or about 60 nm or less. Nanoparticles have typically a longest dimension of about 2 nm or more, about 5 nm or more, about 10 nm or more, about 15 nm or more, or about 20 nm or more.

As used herein, the term “microparticles” refers to particles, larger than nanoparticles, wherein the size of a longest dimension is about 5 μm or less, about 3 μm or less, about 1 μm or less. Microparticles have typically a longest dimension of about 0.25 μm or more, about 0.4 μm or more, about 0.5 μm or more, or about 0.6 μm or more.

As used herein, the terms “ultraviolet-protective agent” or “ultraviolet-protecting agent” refer to agents as used in the art that absorb and/or reflect and/or scatter UV radiation on surfaces exposed to sunlight or any other UV source, so as to reduce the effect of UV radiation on the surface. The surface may be the skin and/or hair of a subject, such as a human subject or a non-human animal. The surface may also be the surface (e.g., an exterior face) of an inanimate object.

When the pigment particles of a UV-protective agent are nanoparticles, they can be solid inorganic crystals that absorb UV radiation. When the pigment particles are microparticles, they can scatter the UV radiation away from the surface they protect. The pigment particles of the inorganic UV-protective agents, whether in nano- or micro-particulate form, can be oxides of metals such as aluminium, barium, bismuth, cadmium, cerium, cobalt, copper, iron, magnesium, manganese, nickel, sodium, titanium, vanadium, zinc, zirconium, and combinations thereof, doped or undoped. The oxides can be in the form of a mono-oxide, a di-oxide, a tri-oxide, or a tetra-oxide, the oxides further optionally in the form of an oxo-anion.

As used herein, the term “doped”, with regards to a crystalline structure (e.g., metal oxide) refer to introducing low amounts of cations, such as metal cations, the cations substituting atoms originally present in the crystalline structure. These cations are termed “dopants” and can be selected from a group consisting of copper, iron, lanthanum and manganese.

If the inorganic pigment is a doped metal oxide, it may comprise from about 90% or even from 95% to about 99.9% mole percentage solid inorganic material and from about 0.1% to about 5% or even 10% mole percentage of a metal cation as a dopant.

In some embodiments, when a relatively transparent composition is sought, the pigments used in the present invention are nanoparticles of an inorganic UV-protective agent selected from the group consisting of barium titanate (BaTiO₃), bismuth lanthanum titanate (Bi₄₋ₓLaₓTi₃O₁₂), bismuth oxide (Bi₂O₃), bismuth vanadate (BiVO₄), bismuth titanate (Bi₄Ti₃O₁₂), bismuth oxybromide (BiOBr), bismuth oxychloride (BiOCl), cerium oxide (CeO₂), titanium dioxide (TiO₂), zinc oxide (ZnO), zinc titanate (ZnTiO₄), zirconium titanate (ZrTiO₄) and combinations thereof, wherein any of these inorganic UV-protective agents may be undoped or doped (e.g., iron-doped bismuth lanthanum titanate (Bi₄₋ₓLaₓTi_{(3-y)}Fe_{y}O₁₂) or iron-doped zinc titanate (Zn₂Ti₍₁₋ₓ₎FeₓO₄)). While these pigment nanoparticles can be used in combination with other nanoparticles of inorganic or organic pigments, they may also provide alone protection from UV radiation (by absorption) over a relatively broad part of the UV range and be therefore considered relatively potent UV-protective agents.

Nanoparticles of pigments can additionally be selected from a group comprising sodium aluminosulfosilicate, aluminum oxide, cadmium sulfoselenide, cerium oxide, cobalt oxide, cobalt titanate, copper oxide, iron oxide, mixed Fe—Mg—Ti oxides, magnesium aluminum oxide, manganese dioxide, manganese ferrite, nickel antimony, manganese vanadium oxide, zirconium oxide, and combinations thereof.

While the above nanoparticles may provide for clear or substantially transparent UV-protective compositions in view of their size, some nano-pigments may additionally impart a color. Pigments which may provide some coloring effect to the composition despite being nanoparticles include, for instance, bismuth vanadate, bismuth titanate, cerium oxide, cadmium sulfoselenide, cobalt oxide, copper oxide, iron oxide, mixed Fe—Mg—Ti oxides and manganese ferrite.

When in microparticulate form, any and all of the above pigments impart a color (including white) and can thus be used in any composition that is designed to provide a color to a relatively opaque product. While microparticles of said pigments may scatter rather than absorb UV radiation, they may nevertheless provide a UV-protective effect.

Alternatively, or additionally, coloring pigment particles can be organic pigments, for example, selected from the group consisting of perylene pigments; phthalocyanine pigments; quinacridone pigments; and imidazolone pigments.

According to some embodiments, the organic or inorganic pigments are selected from the group consisting of the following EU-approved colors for cosmetic use: CI 10006, CI 10020, CI 10316, CI 11680, CI 11710, CI 11725, CI 11920, CI 12010, CI 12085, CI 12120, CI 12370, CI 12420, CI 12480, CI 12490, CI 12700, CI 13015, CI 14270, CI 14700, CI 14720, CI 14815, CI 15510, CI 15525, CI 15580, CI 15620, CI 15630, CI 15800, CI 15850, CI 15865, CI 15880, CI 15980, CI 15985, CI 16035, CI 16185, CI 16230, CI 16255, CI 16290, CI 17200, CI 18050, CI 18130, CI 18690, CI 18736, CI 18820, CI 18965, CI 19140, CI 20040, CI 20470, CI 21100, CI 21108, CI 21230, CI 24790, CI 26100, CI 27755, CI 28440, CI 40215, CI 40800, CI 40820, CI 40825, CI 40850, CI 42045, CI 42051, CI 42053, CI 42080, CI 42090, CI 42100, CI 42170, CI 42510, CI 42520, CI 42735, CI 44045, CI 44090, CI 45100, CI 45190, CI 45220, CI 45350, CI 45370, CI 45380, CI 45396, CI 45405, CI 45410, CI 45430, CI 47000, CI 47005, CI 50325, CI 50420, CI 51319, CI 58000, CI 59040, CI 60724, CI 60725, CI 60730, CI 61565, CI 61570, CI 61585, CI 62045, CI 69800, CI 69825, CI 71105, CI 73000, CI 73015, CI 73360, CI 73385, CI 73900, CI 73915, CI 74100, CI 74160, CI 74180, CI 74260, CI 75100, CI 75120, CI 75125, CI 75130, CI 75135, CI 75170, CI 75300, CI 75470, CI 75810, CI 77000, CI 77007, CI 77266, CI 77267, CI 77268:1, CI 77891, CI 77947, lactoflavine, caramel, capsanthin, capsorubin, beetroot red, anthocyanins, bromothymol blue, bromocresol green, and acid red 195.

According to some embodiments, the pigments are selected from the group consisting of the following US-certified organic colors for cosmetic use: D&C Black No. 2, D&C Black No. 3, FD&C Blue No. 1, D&C Blue No. 4, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, FD&C Red No. 40, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10 and D&C Yellow No. 11.

When the composition is used as a cosmetic product, for the purpose of providing a color (e.g., makeup, foundation etc.), the colored pigments can be combined with additional pigments that blur any skin imperfections or wrinkles, so that the effect of the colored pigments is not masked or attenuated by the original skin tone and condition. Such blurring pigments include, e.g., titanium dioxide (specifically of a rutile crystalline structure) and bismuth oxychloride, which efficiently scatter visible light, thereby imparting whiteness, brightness and opacity when incorporated into a cosmetic composition, serving as a base or background for the skin, on which the color is imparted.

The dimensions of the various particles (biodegradable polymer macroparticles or pigment particles) may be estimated by scanning electron microscope (SEM), transmission electron microscope (TEM) focused ion beam (FIB), and/or by confocal laser scanning microscopy techniques. For instance, scanning electron microscopy may be used for assessment of the planar dimensions, while thickness or length of particles can be determined by focused ion beam FIB technique. Such dimensions can be assessed, for instance, by image analysis of at least one instrumental field of view obtained by suitable microscopic technique and magnification, the at least one field of view comprising at least 10 particles, the longest dimension being the average of the length of the particles so analysed.

If assessing the respective populations of pigment particles or macroparticles is desired, such microscopic measurements are repeated on a number of particles to gain statistical significance. Certain microscopes incorporate image analyser able to readily provide metrics of relevance to the population of particles captured in the relevant field of view. Depending on the microscopy technique, the magnification and the size of the particles under investigation, a field of view may include at least 5 particles, at least 10 particles, or at least 20 particles; and optionally, at most 200 particles, or at most 100 particles, or at most 50 particles. In some embodiments, a field of view includes a number of particles within a range of 5 to 200, 10 to 100 or 20 to 50. In some embodiments, two or more distinct fields of view are being considered to reach the number of particles deemed sufficient to reasonably represent the population.

In other embodiments, the size of the pigment particles, or of the macroparticles of swelled biodegradable polymer matrix, is determined by Dynamic Light Scattering (DLS) or Light Scattering (LS), the former being more suited for relatively smaller particles (e.g., of up to 6 μm) and the latter being more suited for relatively larger particles (e.g., of up to 3.5 mm). In DLS or LS techniques the particles, whether in the sub-micron “nano range” or above micron “micro range” of some pigments or “macro range” of embedding polymer, are approximated to spheres of equivalent behavior and the size can be provided in term of hydrodynamic diameter. DLS and LS also allow readily assessing the size distribution of a population of particles. Such methods, though not exclusive, are preferred to assess if the size distribution of the particles is or not substantially unimodal (i.e. having a single or highly predominant peak).

As used herein in the specification and in the claims section that follows, the terms “long dimension” or “longest dimension” refer to the maximum longest dimension of a particle (such as a polymer flake or a pigment particle) as viewed in a field of view of an image-capturing instrument, such as SEM-FIB.

Distribution results can be expressed in terms of the hydrodynamic diameter for a given percentage of the cumulative particle size distribution, either in terms of numbers of particles (denoted D_{N}) or volumes (denoted D_{V}), and are typically provided for 10%, 50% and 90% of the cumulative particle size distribution. For instance, D50 refers to the maximum hydrodynamic diameter, below which 50% of the sample volume or number of particles, as the case may be, exists. The median diameter by volume is termed D_{V}50, and by number is termed D_{N}50, the latter is also referred to as the “average particle size”. As used herein, a “maximal particle size” refers to the maximum hydrodynamic diameter, below which 99% of the sample of particles exists, either by volume (in which case, termed D_{V}99) or by number (in which case, termed D_{N}99).

When the pigment particles are adapted to provide at least a UV-protective effect by absorption of UV radiation, e.g., being organic or inorganic nanoparticles of a UV-protective agent, at least 50%, at least 55%, at least 60%, at least 65%, at least 70%, at least 75%, at least 80%, at least 85%, at least 90%, at least 95%, at least 97.5%, or at least 99%, per number, per volume, or per surface area, of the organic or inorganic nanoparticles of the UV-protective agent have a cumulative hydrodynamic diameter of up to about 250 nm, up to about 200 nm, up to about 150 nm, up to about 100 nm, up to about 90 nm, up to about 80 nm, up to about 70 nm, or up to about 60 nm. At least 10%, at least 15%, at least 20%, at least 25%, at least 30%, at least 35%, at least 40%, at least 45%, or at least 50% of the pigment nanoparticles may in accordance have a cumulative hydrodynamic diameter of at least about 2 nm, at least about 5 nm, at least about 10 nm, at least about 15 nm, or at least about 20 nm. In its broadest range, 10% of the pigment nanoparticles (D10) may have a hydrodynamic diameter of about 2 nm or more and 99% of the nanoparticles of pigments (D99) may have a hydrodynamic diameter of 250 nm or less, a D10 of 5 nm or more and a D99 of 200 nm or less, a D10 of 10 nm or more and a D99 of 150 nm or less, or a D10 of 15 nm or more and a D99 of 100 nm or less. In preferred embodiments, the cumulative hydrodynamic diameter of the pigment nanoparticles is calculated per volume of the nanoparticles in the population.

When the pigment particles are organic or inorganic pigment microparticles providing at least a coloring effect, at least 50%, at least 55%, at least 60%, at least 65%, at least 70%, at least 75%, at least 80%, at least 85%, at least 90%, at least 95%, at least 97.5%, or at least 99%, per number, per volume, or per surface area, of the pigment microparticles have a cumulative hydrodynamic diameter of up to about 5 μm, up to about 3 μm, or up to about 1 μm. At least 50%, at least 55%, at least 60%, at least 65%, at least 70%, at least 75%, at least 80%, at least 85%, at least 90%, at least 95%, at least 97.5%, or at least 99% of the pigment microparticles may in accordance have a cumulative hydrodynamic diameter of about 0.6 μm or more, about 0.5 μm or more, about 0.4 μm or more, or about 0.25 μm or more. At least 50%, at least 55%, at least 60%, at least 65%, at least 70%, at least 75%, at least 80%, at least 85%, at least 90%, at least 95%, at least 97.5%, or at least 99% of the microparticles may have a hydrodynamic diameter between 0.25 μm and 5 μm, between 0.25 μm and 3 μm, between 0.4 μm and 3 μm, or between 0.4 μm and 1 μm. In preferred embodiments, the cumulative hydrodynamic diameter of the pigment microparticles is calculated per volume of the microparticles in the population.

As readily appreciated the afore-said ranges are logically related and if a population of particles display, for illustration, a D_{V}99 of about 5 μm for microparticles and of about 250 nm for nanoparticles, smaller portions of the same population will only display a smaller cumulative hydrodynamic diameter (e.g., a D_{V}90 of about 2 μm for microparticles and of about 100 nm for nanoparticles and a D_{V}50 of about 1 μm for microparticles and of about 50 nm for nanoparticles). Likewise, the lower end of the size range will only be accordingly smaller.

In some embodiments, the pigment particles have a maximal particle size (D_{V}99) of up to about 5 μm, up to about 3 μm, or up to about 1 μm. Such pigment microparticles may additionally have a maximal particle size of at least about 0.25 μm, at least about 0.4 μm, at least about 0.5 μm or at least about 0.6 μm. In some embodiments, the microparticles have a maximal particle size (D_{V}99) between 0.25 μm and 5 μm, between 0.25 μm and 3 μm, between 0.4 μm and 3 μm, or between 0.4 μm and 1 μm. Such pigment particles typically have at least a coloring effect, but may additionally provide a UV scattering effect.

In other embodiments, the pigment particles have at least a UV-protective effect (e.g., are inorganic nanoparticles of a UV-protective agent), in which case they may have a maximal particle size (D_{V}99) of up to about 250 nm, up to about 200 nm, up to about 150 nm, up to about 100 nm, up to about 90 nm, up to about 80 nm, up to about 70 nm or even up to 60 nm. Such pigment nanoparticles may additionally have a minimal particle size (D_{V}10) of at least about 2 nm, at least about 5 nm, at least about 10 nm, at least about 15 nm, or at least about 20 nm. In some embodiments, the pigment nanoparticles can have a D_{V}10 of 2 nm or more and a D_{V}99 of 250 nm or less, a D_{V}10 of 5 nm or more and a D_{V}99 of 200 nm or less, a D_{V}10 of 10 nm or more and a D_{V}99 of 150 nm or less, or a D_{V}10 of 15 nm or more and a D_{V}99 of 100 nm or less.

In some embodiments, the pigment particles have a unimodal particle size distribution. In alternative embodiments, the particles have at least a bimodal distribution having a first peak (weight/area) representing a first population of particles and a second peak or subsequent peaks representing a second population or subsequent populations of particles, wherein said first peak is larger than said second peak, and optional subsequent peaks.

The pigment particles may, in some embodiments, be uniformly shaped and/or within a symmetrical distribution relative to a median value of the population and/or within a relatively narrow size distribution.

A particle size distribution is said to be relatively narrow if at least one of the following conditions applies:
A) For pigment nanoparticles, the difference between the hydrodynamic diameter of 90% of the particles and the hydrodynamic diameter of 10% of the particles is equal to or less than 200 nm, equal to or less than 150 nm, or equal to or less than 100 nm, or equal to or less than 50 nm, which can be mathematically expressed by: (D90−D10)≤200 nm and so on;
B) For pigment microparticles, the difference between the hydrodynamic diameter of 90% of the particles and the hydrodynamic diameter of 10% of the particles is equal to or less than 2 μm, equal to or less than 1.5 μm, or equal to or less than 1 μm, or equal to or less than 0.5 μm, which can be mathematically expressed by: (D90−D10)≤2 μm and so on; and/or
C) the ratio between a) the difference between the hydrodynamic diameter of 90% of the particles and the hydrodynamic diameter of 10% of the particles; and b) the hydrodynamic diameter of 50% of the particles, is no more than 2.0, or no more than 1.5, or even no more than 1.0, which can be mathematically expressed by: (D90−D10)/D50≤2.0 and so on; and/or
D) the polydispersity index of the particles is equal to or less than 0.4, or equal to or less than 0.2, or even equal to or less than of 0.1, which can be mathematically expressed by: PDI=σ²/d²≤0.4 and so on, wherein σ is the standard deviation of the particles distribution and d is the mean size of the particles, the PDI optionally being equal to 0.01 or more, 0.05 or more, or 0.1 or more.

In some embodiments, the pigment particles are homogeneously dispersed and embedded in the swelled biodegradable polymer matrix macroparticles, such that the surface area of each such pigment particle is fully encased in the swelled polymer matrix macroparticle. Preferably, the pigment particles are sufficiently dispersed within the swelled biodegradable polymer matrix macroparticles so as to prevent or reduce formation of clumps or aggregates of particles to an extent that would affect their intended use.

It is believed that a population of pigment particles which is well dispersed before their embedment in the biodegradable polymer matrix will at least remain in this advantageous state once incorporated according to the present teachings. This can be ascertained by measuring the optical absorption of a same amount of pigment before and after embedment. Similar curves support that the pigment particles remain similarly dispersed, a significant aggregation being otherwise expected to shift the absorption curve towards higher wavelengths, such drift being called a red-shift. But the embedment of the pigment particles and their milling in the swollen polymer may further disperse them, if the pigment particles were added at a secondary particle size greater than their primary particle size. In such case, the absorption curve may shift towards lower wavelengths, displaying a blue-shift.

While some polymers or oils can independently promote the dispersibility of pigments (e.g., polymers having an acidic moiety), a dispersant may be added to the present compositions to disperse (or facilitate the dispersion of) the particles either in a liquid carrier before their incorporation into the swollen thermoplastic biodegradable polymer or during their co-milling therewith.

As the oil carrier serving as a dispersing medium for the particles, may additionally serve as the swelling agent (or one of the swelling agents) of the biodegradable polymer, the oil carriers may be any one of the previously described polar oils capable of serving as swelling agents (e.g., a synthetic, vegetal or essential oil having a calculated HLB value of at least 3). But in contrast to the relatively polar swelling agents, the oil carrier can alternatively be non-polar. Thus, an oil carrier may have calculated HLB value within a range of −10 to 25, to accommodate relatively non-polar oils, in a range of −10 to less than 3, and relatively polar oils, in a range of 3 to 25.

Relatively non-polar oil carriers, which can have calculated HLB values of less than 3, may be selected from: non-polar fatty esters (e.g., propylheptyl caprylate, ethylhexyl stearate, cetearyl isononanoate, caprylyl caprylate, dicaprylyl carbonate, hexyl laurate, hexyldecyl stearate, oleyl erucate and C₁₂-C₁₅ alkyl benzoate); C₁₀-C₃₀ hydrocarbons (e.g., diethylhexylcyclohexane, as commercially available, for instance, from BASF as Cetiol® S, and a mixture of undecane and tridecane, as commercially available, from BASF as Cetiol® Ultimate) and isoparaffins, as commercially available, for instance, from Exxon Mobil Chemical as Isopar™ L, Isopar™ M and Isopar™ H); non-polar fatty alcohols (e.g., octyldodecanol and hexyldecanol, as well as their mixtures with acid esters, e.g., a mixture of hexyldecanol and exyldehcyl laurate); and non-polar ethers (e.g., diphenyl ether, polyoxypropylene stearyl ether or dioctyl ether, the latter two being commercially available, for instance, from BASF as Cetiol® E and Cetiol® OE, respectively).

In some embodiments, the pigment oil carrier is selected from: a saturated or unsaturated acid mono or diester, particularly butyl lactate, dimethyl glutarate, dimethyl maleate, dimethyl methyl glutarate, ethyl lactate and lactic acid isoamyl ester; a fatty acid ester, particularly 2-ethylhexyl lactate, acetyl tributyl citrate, acetyl triethyl citrate, acetyl triethyl hexyl citrate, allyl hexanoate, benzyl benzoate, butyl butyryl lactate, a mixture of caprylyl caprylate and caprylyl caprate dibutyl adipate, dibutyl maleate, dibutyl sebacate, diethyl succinate, glyceryl trioctanoate 1-menthyl lactate, lauryl lactate, n-pentyl benzoate, PEG-6 caprylic/capric glycerides, triacetin and triethyl citrate and; an aromatic alcohol, particularly benzyl alcohol; an ether, particularly diethylene glycol diethyl ether and dimethyl isosorbide; and an aldehyde, particularly cinnamaldehyde (as can be found in cinnamon bark oil).

An oil suitable for the present compositions and methods, as a swelling agent for the polymers and/or as an oil carrier in which the pigments can be dispersed, can be substantially non-volatile at a temperature at least about 50° C. Such lack of volatility can prevent or reduce shrinkage of the macroparticles of swollen polymer over time to an extent leading to a loss of activity, as a result, for illustration, of a breakdown of the embedded dispersion.

A swelling agent, an oil carrier, or any other liquid as may be used in the present disclosure is deemed non-volatile if having a vapor pressure of less than 40 Pascal (Newton per square meter) at a temperature of about 25° C. Such values are typically provided by the manufacturer of the liquid, but can be independently determined by standard methods, such as described in ASTM D2879, E1194, or E1782 according to the range of the vapor pressure.

This however is not essential as some volatility can be tolerated or even desired. The relative volatility of an oil that may serve as dispersing medium for pigment particles and/or as swelling agent for biodegradable polymers can affect, if at all, the preparation of the composition, as oil might need to be added during the dispersion and/or swelling to compensate for the amount of liquid that may evaporate in the process. Alternatively, or additionally, the preparation can be performed at a pressure higher than ambient, so as to sufficiently reduce or prevent evaporation of a relatively volatile oil. It is believed that while relatively volatile oils may partially escape the end-product of the present method, namely the macroparticles of swelled biodegradable polymer matrix, at this stage, a relatively volatile swelling agent may not entirely disappear from the polymer matrix, remaining in an amount sufficient for the pigment particles embedded therein to remain dispersed as discrete particles. It is believed that a swelling agent, even if relatively volatile, may be present in a swollen macroparticles at a weight concentration of at least 0.5% by weight of the macroparticles, at least 1 wt. % or at least 2 wt. %. A swelling agent, an oil carrier, or any other liquid as may be used in the present disclosure is deemed relatively volatile, but still suitable, if having a vapor pressure between 40 Pa and 25 kPa, between 50 Pa and 10 kPa, between 60 Pa and 5 kPa, or between 100 Pa and 1 kPa, at a temperature of about 25° C.

In some embodiments, it may be desired for the liquid in which the macroparticles may be placed following their preparation (e.g., by replacement of the medium surrounding the flakes) to be highly volatile. Such volatility facilitates the elimination (e.g., by evaporation) of the liquid medium from the final composition, and consequently increases the relative concentration of pigment particles in the composition. Importantly, such relatively higher concentrations of pigments may exceed the limits that could be reached in traditional dispersions in which particles would naturally re-agglomerate and/or precipitate above a certain concentration (typically if exceeding 20-30 wt. %). A liquid is deemed highly volatile, and capable of being eliminated so as to accordingly increase the relative concentration of pigment particles, typically if having a vapor pressure of 1 kPa or more, 2.5 kPa or more, 5 kPa or more, or 10 kPa or more, at a temperature of about 25° C.

Exemplary volatile liquids include: short C₁-C₁₀ alcohols, such as ethanol and isopropyl alcohol, short ketones, such as acetone and methyl ethyl ketone, and short volatile oils, such as linear or cyclic silicone oils, for instance trisiloxane, dimethicone, octamethyl-cyclotetrasiloxane and decamethylcyclopentosiloxane.

According to some embodiments, a dispersant is utilized in the present compositions or methods for preparing the same. Such a dispersant may be any additive that increases the dispersibility of the pigment particles in at least one of the oils (swelling agent or carrier) to be added to the (optionally pre-swollen) biodegradable polymer and further embedded into the swelled polymer matrix macroparticles. The dispersant needs to be compatible with the biodegradable polymer, so that dispersant-enveloped particles are incorporated into the polymer without any phase separation. The dispersant also needs to be compatible with and soluble in the oil carrier, within which the particles are dispersed, if not added as a dry powder. In some embodiments, the dispersant comprises a hydrocarbon portion, which interacts with the biodegradable polymer, and a carboxylic acid function which interacts with oxides on the surface of the pigment particles, thus rendering the dispersant-laden or dispersant-enveloped pigment particles dispersible in the polymeric matrix. In some preferred embodiments, the dispersant comprises fatty acids or polymers thereof.

In some embodiments, the oil soluble dispersant has a positive Hydrophilic-Lipophilic Balance (HLB) value of no more than 9, no more than 6 or even no more than 3. In some embodiments, the HLB of the dispersant is about 2.5.

In some embodiments, the weight per weight ratio of dispersant to pigment particles being dispersed therewith, is between 2:1 and 1:100, between 1:1 and 1:100, between 1:1 and 1:90, between 1:1 and 1:70, or between 1:3 and 1:50. In a particular embodiment, when the pigment particles are nanoparticles, the weight per weight ratio of dispersant to pigment particles is of about 1:10.

Non-limiting examples of suitable dispersants include any of the Pelemol esters, available commercially from Phoenix Chemicals, Overland Park, Kansas, USA: Pelemol® PHS-8 (polyhydroxystearic acid); Pelemol® BIP-PC (butylphthalimide and isopropylphthalimide); Pelemol® C₂₅EH (C₁₂-C₁₅ alkyl ethylhexanoate); Pelemol® CA (cetyl acetate); Pelemol® 899 (isononyl isononanoate and ethylhexyl isononanoate); Pelemol® 168 (cetyl ehtylhexanoate); Pelemol® 89 (ethylhexyl isononanoate); Pelemol® 3G22 (polyglyceryl-3 beherate); Pelemol® D5R1 (ethyl isononanoate and cetyl dimethicone); Pelemol® D5RV (propanediol dicaprylate/caprate and diisostearyl malate); Pelemol® D899 (PPG-26 dimer dilinoleate copolymer and isononyl isononanoate and ethylhexyl isononanoate); Pelemol® DD (dimer dilinoleyl dimer dilinoleate); Pelemol® DDA (diethylhexyl adipate); Pelemol® DO (decyl oleate); Pelemol® DP-72 (dipentaerythirityl tetrabehenate/poyhydroxystearate-lanolic substitute); Pelemol® EE (octyldodecyl erucate); Pelemol® G7A (glyceryl-7 triacetate); Pelemol® GMB (glyceryl behemate); Pelemol® GMR (glyceryl ricinoleate); Pelemol® GTAR (glyceryl triacetyl ricinoleate): Pelemol® GTB (tribehenin); Pelemol® GTHS (trihydroxystearin); Pelemol® GTIS (triisostearin); Pelemol® GTO (triethylhexanoin); Pelemol® ICB (isocetyl behenate); Pelemol® II (isostearyl isostearate); Pelemol® IN-2 (isononyl isonanoate); Pelemol® ISB (isostearyl behenate); Pelemol® ISHS (isostearyl hydroxystearate); Pelemol® ISNP (isostearyl neopentanoate); Pelemol® JEC (triisostearin/glyceryl behenate); Pelemol® MAR (methyl acetyl ricinoleate); Pelemol® NPGDD (neopentylglycol/dicaprate/dicaprylate); Pelemol® OL (oleyl lactate); Pelemol® OPG (ethylhexyl pelargonate); Pelemol® P-49 (pentaerythrityl teraisononanoate); Pelemol® P-810 (propanediol dicaprylate/caprate); Pelemol® P-1263 (polyglycerol-10 hexaoleate and polyglyceryl-6 poyricinoleate); Pelemol® PTIS (pentaerythrityl tetraisostearate); Pelemol® PTL (pentaerythrityl terralaurate); Pelemol® PTO (pentaerythrityl tetraethylhexanoate); Pelemol® SPO (cetearyl ethylhexanoate; Pelemol® TDE (tridecyl enucate); Pelemol® TGC (trioctyldodecyl citrate); Pelemol® TMPIS (trimethylolpropane triisostearate); Pelemol® TMPO (trimethylopropane triethylhexanoate); Pelemol® TT (tribehenin and caprylic acid/capric triglyceride); Pelemol® VL (dimer dilinolelyl dier dilinoleate and triisostearin). Additional polyhydroxystearic acid dispersants include Dispersun DSP-OL100 and DSP-OL300, commercially available from Innospec Performance Chemicals. Further suitable dispersants include octyldodecyl/PPG-3 myristyl ether dimer dilinoleate (such as commercially available as PolyEFA from Croda Inc.). Oleic acids may also be used as dispersants for the purpose of the present invention.

In particular embodiments, the dispersant is polyhydroxystearic acid (e.g., as commercially available under trade names Pelemol® PHS-8, Dispersun DSP-OL100 or DSP-OL300).

In some embodiments, the dispersant associated with the pigment particles to ensure their adequate dispersion in an oil carrier before their incorporation into the biodegradable polymer matrix, is the sole dispersant used in the composition.

The pigment particles are preferably present in the swelled biodegradable polymer matrix macroparticles at a concentration from about 0.01 to about 400 wt. %, from about 0.05 to about 300 wt. %, from about 0.1 to about 200 wt. %, from about 0.1 to about 100 wt. %, from about 0.1 to about 80 wt. %, from about 0.1 to about 60 wt. %, from about 0.1 to about 40 wt. %, from about 0.3 to about 30 wt. %, or from about 0.5 to about 25 wt. %, by weight of the thermoplastic biodegradable polymer. In some embodiments, the pigment particles are present in the swelled biodegradable polymer matrix macroparticles at a concentration of about 1 wt. %, about 5 wt. %, about 10 wt. %, about 20 wt. %, about 50 wt. %, about 70 wt. %, about 100 wt. %, about 150 wt. %, or about 200 wt. % by weight of the thermoplastic biodegradable polymer.

While the macroparticles can be isolated to constitute the entire composition, in some embodiments, the macroparticles of swollen biodegradable polymer embedding the pigment (e.g., inorganic) particles are present at a concentration of no more than 50 wt. %, no more than 40 wt. %, no more than 30 wt. %, or no more than 20 wt. % by weight of the total composition disclosed herein.

In some embodiments, the pigment particles are present in the macroparticles at a concentration from about 0.001 to about 80 wt. %, from about 0.001 to about 70 wt. %, from about 0.005 to about 60 wt. %, from about 0.005 to about 50 wt. %, from about 0.01 to about 40 wt. %, from about 0.1 to about 30 wt. %, from about 0.5 to about 20 wt. %, from about 0.7 wt. % to about 15 wt. %, or from about 0.7 to about 10 wt. % by weight of the swelled biodegradable polymer matrix macroparticles.

In some embodiments, the pigment particles and the thermoplastic biodegradable polymer constitute up to 95 wt. %, up to 90 wt. %, up to 85 wt. %, or up to 80 wt. % of the macroparticles of swelled biodegradable polymer matrix, the remaining at least 5 wt. %, 10 wt. %, 15 wt. %, or 20 wt. %, respectively, being contributed by the swelling agent(s), and optional oil carrier(s), dispersant, and/or like additives.

As the pigment particles, e.g., nanoparticles of UV-protective agents, typically have a density higher than the density of the thermoplastic biodegradable polymer, the relative proportion of the pigment particles to polymer on a volume per volume (v/v or vol. %) basis is limited. For reference, the density of the UV-protective agents herein disclosed, which may be further affected by the presence and degree of doping, ranges from about 3 g/cm³ to about 10 g/cm³ (e.g., −4.23 g/cm³ for titanium dioxide), while thermoplastic biodegradable polymers can have a density around 1 g/cm³ (e.g., 1.43 g/cm³ for polylactic acid).

When preparing pigment-containing products, such as described in the present invention, it can be desired to have a high amount of pigment, whether for UV-protecting purposes or for coloration purposes as well. However, at the same time, there is an upper limit to the amount of pigments that can be embedded in the biodegradable polymeric matrix, while retaining satisfactory dispersion without undesired pigment agglomeration. Hence, the weight ratio of biodegradable polymer to the pigment in the composition needs to be balanced so that on one hand, there is no pigment agglomeration, and on the other hand, the amount of pigment is enough for the cosmetic or coating purposes being sought. In some embodiments, the pigment particles to biodegradable polymer ratio is at least about 1:100, at least about 1:50, at least about 1:20, at least about 1:10, at least about 1:7, at least about 1:5, at least about 1:3, or at least about 1:1, on a weight per weight basis. In some embodiments, the ratio of the pigment particles to biodegradable polymer is at most about 20:1, at most about 15:1, at most about 10:1, at most about 8:1, at most about 6:1, at most about 4:1, or at most about 2:1 on a weight per weight basis. In some embodiments, the pigment particles to biodegradable polymer weight per weight ratio is within a range of 1:100 to 20:1, within a range of 1:50 to 10:1, within a range of 1:20 to 8:1, within a range of 1:10 to 6:1 or within a range of 1:5 to 4:1.

When relatively high amounts of pigments (e.g., 30 wt. % or more) are to be embedded, and in particular when the pigment particles are relatively small (increasing the surface are of the particles to be dispersed), the stock of dispersed particles may develop a viscosity to a level hampering proper embedment in the swollen polymer. In such cases, it may be desirable to select the particles oil carrier and/or the polymer swelling agent(s) to have sufficiently low viscosity and/or viscosity thinning properties. In preferred embodiments, the previously listed oil carriers are compatible with relatively high loading of pigment particles without detrimental raise in viscosity preventing their embedment.

Alternatively, or additionally, a viscosity reducer may be added to the composition. To be suitable, a viscosity reducer should be compatible with the pigment particles' oil carrier, the polymer swelling agent, as well as with the pigment and its dispersant (if present). Moreover, the viscosity reducer should maintain the integrity of the macroparticles of swollen polymer, being selected so as to not cause a dissolution of the biodegradable polymer by more than 0.1 wt. %. In particular embodiments, the viscosity reducer can be selected from: hexylene glycol, sorbitan trioleate or oleyl amine.

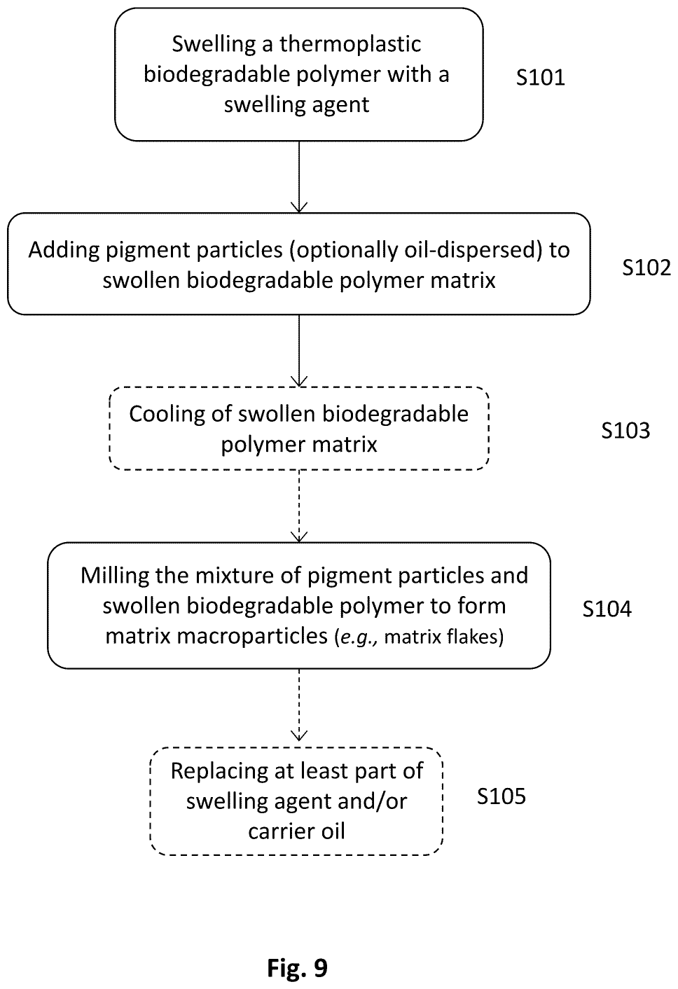

The present invention further provides methods for the preparation of compositions as disclosed herein and the following materials refer inter alia to the substances extensively detailed with respect to the compositions. The steps of one such method are schematically illustrated in FIG. 9.

In a first step (S101), a thermoplastic biodegradable polymer, having at least one of a melting temperature (Tm), a softening temperature (Ts) and a glass transition temperature (Tg) of 200° C. or less, is swelled by combining the polymer with at least one non-aqueous polar swelling agent having a calculated Hydrophilic-Lipophilic Balance (HLB) value of at least about 3. The swelling encompasses mixing the combination of the thermoplastic biodegradable polymer and the swelling agent(s) to provide a paste of polymer matrix, wherein the thermoplastic biodegradable polymer is homogeneously swelled with the swelling agent(s) either as a single mass or as morsels of swelled polymer matrix.

While swelling could take place at ambient temperature and pressure, in some embodiments, the swelling is performed while heating the combination to a swelling temperature from about 0° C. to about 20° C., or from about 0° C. to about 30° C., from about 0° C. to about 40° C., from about 0° C. to about 50° C. above the melting or softening temperature of the thermoplastic biodegradable polymer. Alternatively, the optional heating is performed at the softening temperature or above (e.g., by no more than 50° C.) the softening temperature of the combined polymers and/or mixture thereof with the oil(s), or any other agent acting as plasticizer for the thermoplastic biodegradable polymer(s), such combination constituting or forming a portion of the swelling mixture which typically displays characterizing temperatures below the native unswollen polymer. Swelling need not be performed at a single temperature and may for instance be performed over a range of temperatures, which may decrease over time as the polymer is swelling and its thermal behavior typically shifting to display lower characterizing temperatures, e.g., a lower softening point.

Depending on their chemical and/or physical properties, thermoplastic biodegradable polymers, which are known to persons skilled in the art and identified as such by their suppliers, can be characterized either by their melting point or temperature (Tm), by their softening point or temperature (Ts), or by their glass transition temperature (Tg). Such values are typically provided by the suppliers of the polymers and can be determined according to standard procedures, typically using Differential Scanning Calorimetry (DSC), such as described in ASTM 3418, ISO 3146, ASTM D1525 or ISO 11357-3.

In some embodiments, the swelling temperature is at least about 30° C., at least about 40° C., at least about 50° C., at least about 60° C., at least about 70° C., or at least about 80° C. In some embodiments, the swelling temperature is at most about 150° C., at most about 140° C., at most about 130° C., or at most about 120° C. In some embodiments, the swelling temperature is within a range of 30° C. to 150° C., within a range of 50° C. to 150° C., within a range of 60° C. to 140° C., or within a range of 80° C. to 120° C.

The swelling can additionally, or alternatively, be performed at an elevated pressure above ambient pressure of about 100 kPa. The swelling pressure can be above about 125 kPa, above about 150 kPa, above about 175 kPa, above about 200 kPa, above about 250 kPa, or above about 300 kPa. Typically, the swelling pressure does not exceed 10 megaPascal (MPa). Such elevated pressure may allow the acceleration of the swelling process, or may allow the swelling process to occur at lower temperature than might be required at atmospheric pressure. The duration of swelling may understandingly depend on the biodegradable polymer(s) being swelled, the swelling agent(s) being used, the swelling conditions (e.g., temperature, pressure, and/or agitation), and the desired extent of swelling. The swelling period can be of at least 10 minutes, at least 20 minutes, at least 30 minutes, at least 2 hours, at least 4 hours, at least 6 hours, at least 12 hours, or at least 18 hours. While for the assessment of swellability, a swelling step can be carried out for up to 4 days, the swelling period can be shorter, and for instance be 3 days or less, or 2 days or less.

While any desired degree of swelling can be achieved in a swelling step as above described, in some embodiments, the swelling is performed at at least one swelling temperature and/or under at least one swelling pressure for a swelling period so as to obtain a swelled polymer matrix including at least 5 wt. % of swelling agent per weight of polymer.

In some embodiments, the swelling of the biodegradable polymer by the swelling agent(s) is of at least 10 wt. %, at least 20 wt. % or at least 40 wt. %, as observed by weight gained by the polymer as compared to its initial unswollen weight.

Swelling can be performed under ongoing agitation or mixing, for instance in a double planetary mixer.

In a next step (S102), pigment particles (e.g., UV-protective agent nanoparticles) are added to the swollen polymer matrix paste obtained in step S101. The pigments can be added as supplied by the manufacturer, either as a dry powder or as a liquid dispersion, provided the liquid (which may include an oil and/or a dispersant) is compatible with the biodegradable thermoplastic polymer to which the pigment particles are to be added.

Suitable pigment particles, if commercially available or prepared in a medium other than an oil according to the present teachings, can be transferred to such an oil vehicle by any compatible method able to maintain the desired particle size and dispersibility of the particles. For instance, if provided in an aqueous medium, the medium can be removed by evaporation, or the pigment particles can be freeze-dried or any other such method known to the skilled person, as long as the dried particles can readily redisperse in a desired oil. Optionally the redispersion of the particles in the oil (or mixture of oils) of interest can be performed following the addition of an oil-compatible dispersant and the performance of a dispersing or size-reducing step, as described below, decreasing the amount of agglomerates that may form during the change of media.

In some embodiments, the pigment particles being added are separately prepared by dispersing the pigment particles in an oil carrier so as to preliminarily disperse them ahead of mixing with the polymer. A dispersant can be added at this stage to facilitate the dispersion of the pigment particles in the oil carrier, but could have alternatively (or additionally) be added to the polymer and its swelling agent(s) in step S101. Dispersing techniques are known, and the skilled person can select dispersing conditions providing pigment particles of desired size (e.g., as described above). Example of dispersing equipment (capable of deagglomerating pigments from relatively large secondary particles to smaller ones and optionally down to primary particle size) includes sonicators, high-shear homogenizers, and attrition ball mills.

In a next step (S103), the swelling mixture obtained in step S102 comprising the swollen biodegradable polymer matrix and the pigment particles (optionally in presence of a dispersant) may optionally be allowed to cool or actively cooled to a temperature below the Tm or the Ts of the swelled polymer or the swelling mixture. This step may be required if the swelling temperature or a temperature reached following the addition of the pigment particles, is too high for subsequent processing. The cooling, if needed, should maintain the swelling mixture malleable enough for embedment of the pigment particles, without being too soft to an extent preventing effective milling into separate macroparticles of swelled polymer. However, if the polymer swelling occurs at temperatures low enough for the swelled polymer and the swelling mixture to remain sufficiently relatively solid to be used “as is” in subsequent processing, then no such cooling is required (hence, step S103 is marked by a dashed line in FIG. 9).

In some embodiments, the swollen biodegradable polymer matrix or the swelling mixture is allowed to passively cool or is actively cooled to 50° C. or less, 40° C. or less, or 30° C. or less, the temperature of subsequent milling (hence of cooling, if applied) being typically no less than room temperature (about 23° C.). Cooling can be performed using any suitable equipment, such as transiently transferring to a cooler, blowing air over the materials to be cooled or their containers, or using an equipment including a cooling jacket with a circulating coolant.

To the extent that the biodegradable thermoplastic polymer has a glass transition temperature, the swollen biodegradable polymer matrix or the swelling mixture are preferably milled above such Tg, thus cooling, if needed, seeks to be to a temperature higher than the glass transition temperature of the polymer, in order to maintain its structural integrity. The glass transition temperature may be provided by the suppliers of the polymers, but can also be determined by routine experimentation according to methods known to the skilled persons, for instance by DSC, such as described in ASTM E1356.

While in the illustrative figure, the optional cooling has been described as a separate step taking place after the addition of pigment particles, it can alternatively take place as part of the addition of the pigment particles, for instance if they are added at a temperature achieving the cooling effect, or before the addition of the pigment particles, either as a separate step after the swelling step S101, or as part of the swelling step. For instance, the swelling can be performed under controlled ramping down temperatures and/or the ongoing mixing of the swelling thermoplastic biodegradable polymer may gradually lower the temperature of the mix.

The milling step (S104) serves to size reduce the swollen polymer matrix into swelled biodegradable polymer matrix macroparticles, while dispersing and embedding the pigment particles in the swelled biodegradable polymer matrix macroparticles.

Regardless of the need for a separate cooling step S103 and/or its chronology with respect to the other steps, the swelling mixture is typically at a temperature lower than its Tm or Ts during its milling. Additionally, to the extent that the polymer has a Tg, the milling temperature is higher than a Tg of the swelling mixture, and typically of at least 20° C. Such a temperature, expected to improve the efficiency of the milling step, can be maintained during the entire milling or during at least a part of the milling step, and can thus be referred to as the milling temperature.

The amount of swollen biodegradable polymer that can be milled with a desirable amount of pigment particles, may depend upon the milling system being used in step S104, more energetic ones generally enabling higher polymer and/or pigment concentrations. Preferably, a suitable milling system is capable of applying both shear force and impact force, however a similar effect can be achieved by first embedding the pigments with shear (e.g., with a planetary mixer, or an extruder) then shaping the polymer mass with the embedded pigments with impact forces (e.g., an attritor ball milling equipment, which also provides shear force). The milling conditions, which may additionally depend on the elected milling process (e.g., temperature, type of media mill, type of beads, speed of rotors, duration of milling, and the like factors) may also affect the relative amounts of polymer and pigments that may be found in the macroparticles, their degree of dispersion, their respective size, and/or the shape of the macroparticles being obtained.

The duration of milling may understandingly depend on the swelling mixture being milled, on the milling equipment being used, on the milling conditions, and the desired extent of milling. The milling period can be of at least 10 minutes, at least 20 minutes, at least 30 minutes, at least 1 hour, at least 2 hours, at least 4 hours, at least 6 hours, at least 12 hours, or at least 18 hours. Typically, the milling period does not exceed 2 days or 1 day.

In some embodiments, upon completion of the milling step, substantially all pigment particles are embedded in the macroparticles of the swelled biodegradable polymer matrix. In particular embodiments, the ratio between a number of free non-embedded pigment particles and a number of macroparticles of swelled biodegradable polymer matrix is at most 10:1, at most 5:1, a most 2:1, at most 1:1, at most 1:2, at most 1:5, at most 1:10, or at most 1:20, as can be measured per unit area of a field of view of a microscope.

While in FIG. 9, the illustrative method of preparation of macroparticles of the present invention have been described as a set of particular distinct steps, such steps need not be carried out separately or separated according to the above outlines. For illustration, some equipment (e.g., an extruder) can be suitably used to achieve the compounding of all materials, the swelling of the biodegradable polymer with the at least one swelling agent, the dispersion and the embedment of the pigment particles in the swelling polymer, and the shaping of the compounded mass into macroparticles at once. In other words, steps separately drawn as S101 to S104 in the figure being united into a single step. Alternatively, other equipment may provide for different steps, such as compounding all materials together, achieving swelling, embedding and dispersion of the pigment particles in a first step, then comminuting the compounded mass in a second step, so as to obtain the macroparticles. This alternative two-steps method can be carried out for instance in a double planetary mixer for the compounding step (e.g., S101 to S103) and in a bead mill attritor for the comminuting step (S104).

Regardless of the specific steps used to prepare the swelled biodegradable polymer matrix macroparticles embedding the dispersed pigments, but optionally depending on the equipment being used, the resulting macroparticles may have any suitable shape and may, for example, be in the form of flakes, rods, or spheres.

In some preferred embodiments, at least 50% of the swelled biodegradable polymer matrix macroparticles are flakes. It is believed that flakes provide better packing and coverage when applied on a surface, e.g., to provide a uniform coloring or protect the surface from a harmful effect of UV irradiation.

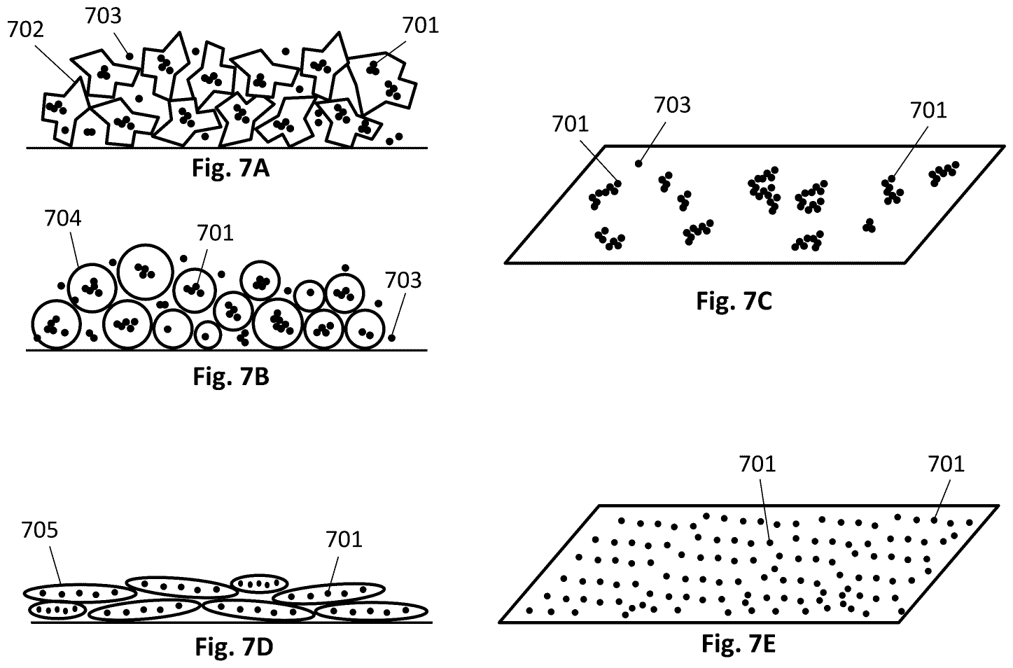

This is illustrated in FIG. 7, where panels A and B schematically show a cross-sectional view of examples of conventionally prepared particles-containing polymers on a target surface. In this example, the pigment particles 701 (e.g., nanoparticles of a UV-protective agent), even if somehow entrapped in the polymer by typical agitation techniques, generally reside in relatively large and/or amorphous chunks of polymers 702. By such method, the particles are unlikely to be uniformly dispersed within the polymer matrix. Moreover, the conventional relatively large chunks would not be expected to be evenly spread on the surface to be protected, due to their disadvantageous relatively bulky shape. This is further illustrated in panel B, where the pigment particles 701 are embedded in polymer particles processed to assume a spherical shape 704, e.g., by suspension polymerization, which makes their arrangement on a surface and their uniform coverage of a surface even more challenging, as each sphere could prospectively have only one point of contact with the surface. More importantly, such method may result in incomplete entrapment of the pigment particles, some remaining out of the macroparticles, and is not expected to form spheres containing evenly dispersed pigment particles.

Furthermore, free particles 703 may be present outside of conventionally prepared polymeric matrix macroparticle, either due to poor embedment or to their migrating out of the polymeric matrix.

Arrangement of the polymeric matrix particles, as depicted in panels A and B would result in the particles remaining on the target surface as irregularly distributed clusters, as schematically illustrated in the perspective top view of panel C. Understandingly, such distribution of the pigment particles 701 leaves uncovered areas. When particles of a UV-protective agent are used, such uncovered areas result in them being unprotected. When pigment particles are used, e.g., in a make-up product, such non-uniform coverage could yield a visual result that is unsatisfactory. Panel C also illustrates the presence of the free pigment particles 703, which, as mentioned above, is undesired.

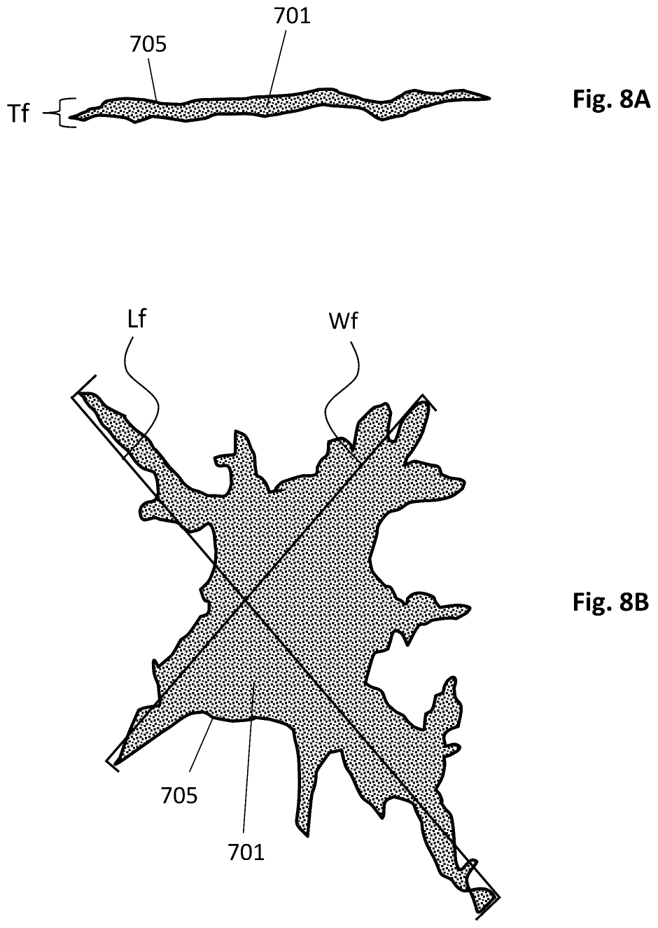

Panel D of FIG. 7 schematically illustrates how particles 701 may be embedded and distributed in swelled biodegradable polymer matrix macroparticles, such as flakes 705, and how the latter may be suitably applied to a target surface, by virtue of their relatively flat shapes. Embedment of the pigment particles in the polymeric matrix, as described in the present invention, results in little or no free pigment between the flakes. The schematic top view of the same, shown in panel E, illustrates how compositions of the invention are expected to provide at least one of a more uniform distribution of particles within the matrix elements, a more uniform coverage of the target surface by the swelled polymer matrix macroparticles, and consequently, a more uniform distribution of the pigment particles on the target surface, and improved protection against UV-radiation (in case the particles are of a UV-protective agent). In some embodiments, the matrix flakes according to the present teachings have an irregular contour, including for instance relatively narrow appendices, such as tentacles, extending or protruding from a relatively broad body. Such matrix flakes can also be referred to as “tentacular flakes”, which will be further described below in reference with FIGS. 8A and 8B.

As used herein, the term “flake” refers to a particle, in particular a macroparticle, having a flake length (Lf), a flake width (Wf), and a flake thickness (Tf), wherein the flake aspect ratio (Rf) is defined by:

Rf=(Lf*Wf)/Tf²

As used herein in the specification and in the claims section that follows, the term “particle length”, “flake length”, or “Lf”, is used generally (and particularly within the context of a “dimensionless flake aspect ratio”), to refer to a maximum length of a particle in its long direction. Perpendicular to Lf (and the like) is measured the “particle width”, “flake width”, or “Wf”, such that Wf<Lf. The term “flake thickness”, or “(Tf)”, at least within the context of a “dimensionless flake aspect ratio”, or “(Rf)”, refers to a maximum distance through a particle, which is orthogonal to both respective lines defining the particle length, or flake length (Lf), and the particle width, or flake width (Wf), such that Tf<Wf (and jointly, Tf<Wf<Lf).

Lf, Wf and Tf may be quantitatively evaluated from a field of view image (e.g., from a “footprint” of the flake or particle) of a suitable image-capturing instrument, such as SEM-FIB.

While selecting a representative particle, or a group of representative particles, which may accurately characterize various properties of the particle population, it will be appreciated that a more statistical approach may yet more accurately characterize such properties. Thus, in some embodiments of the present disclosure, various dimensional properties, including the dimensionless aspect ratio of the particles, may be determined by analysing, in its entirety, a representative field of view of the relevant image-capturing instrument(s) (e.g., SEM-FIB). Such field of view may also be referred to as an “instrumental field of view”. Typically, the magnification of any appropriate instrument (e.g., microscope, DLS) is adjusted such that at least 10 particles, at least 20 particles, or at least 50 particles are disposed within a single instrumental field of view. By way of example, the dimensionless flake aspect ratio for a group of particles may be volume-averaged, surface-area averaged, or number averaged.

In some embodiment, the flake aspect ratio (Rf), and/or an average thereof, is at least about 5, at least about 10, at least about 15, at least about 20, at least about 25, at least about 30, at least about 50, at least about 100, at least about 150, at least about 250, or at least about 500, and optionally, at most about 2,000, at most about 1,500, or at most about 1,000. Said flake aspect ratio can be determined on a representative group of flakes, the group consisting of at least 10 flakes. In some embodiments, the representative group contains at least 20 flakes or at least 50 flakes, and the representative group comprising at least 10 flakes is gathered by studying three or more samples (e.g., three or more distinct fields of view for microscopic analysis).

According to some embodiments, the flake aspect ratio (Rf), and/or an average thereof, is within a range of about 5 to about 2,000, about 10 to about 1,000, about 12 to about 500, about 12 to about 200, or about 15 to about 100.

In some embodiments, at least 50% of the swelled biodegradable polymer matrix macroparticles have a long dimension (or a flake length Lf) of up to about 50 micrometres (μm), up to about 25 μm, up to about 10 μm, or up to about 5 μm, and optionally at least about 0.25 μm, at least about 0.5 μm, at least about 1 μm, at least about 1.5 μm, or at least about 2 μm.

In some embodiments, the width of a flake, or Wf, does not exceed its length (Lf), and can be of at most about 40 μm, at most about 20 μm, at most about 10 μm, or at most about 5 μm, as long as Wf≤Lf. In some embodiments, the width of a flake Wf is at least about 0.2 am, at least about 0.4 μm, at least about 0.8 μm, or at least about 1 μm.

According to some embodiments, the flake thickness (Tf) of the macroparticle is at most about 1 μm, at most about 500 nm, at most about 400 nm, at most about 350 nm, at most about 300 nm, at most about 275 nm, at most about 250 nm, or at most about 225 nm, and optionally at least about 50 nm, at least about 75 nm, at least about 100 nm, at least about 125 nm, at least about 150 nm, or at least about 175 nm.

It should be noted that in order for a pigment particle to be successfully dispersed and embedded in the swelled biodegradable polymer matrix macroparticle, the smallest dimension of the matrix macroparticles (e.g., a flake thickness Tf) should preferably be at least two-fold, four-fold, six-fold, eight-fold or even one order of magnitude greater than the length of the pigment particles.

The macroparticles of swelled biodegradable polymer matrix may have any suitable aspect ratio, i.e., a dimensionless ratio between the longest dimension in the largest plane projecting from the particle and a smallest dimension in a direction orthogonal to said plane.

Such dimensions can be assessed on a number of representative macroparticles by methods known in the art, such as microscopy, including in particular by scanning electron microscope SEM (preferably for the planar dimensions) and by focused ion beam FIB (preferably for the thickness). Macroparticles having an almost spherical shape are characterized by an aspect ratio of approximately 1:1, whereas flake-like particles can have an aspect ratio, or “ASP” (e.g., between their length and their thickness, ASP=Lf/Tf) of 100:1 or more. Though not limiting, the macroparticles of swollen biodegradable polymer matrix according to the present teachings can have an aspect ratio (or average aspect ratio considering a population of matrix flakes, ASP_{avg}=Lf_{avg}/Tf_{avg}) of about 1,000:1 or less, about 500:1 or less, about 100:1 or less, about 75:1 or less, about 50:1 or less, about 25:1 or less, or even of about 10:1. In some embodiments, the matrix flakes according to the present teachings may have an aspect ratio (or average aspect ratio) of at least 2:1, at least 3:1, at least 5:1, at least 10:1, at least 25:1, at least 40:1, or at least 70:1. In some embodiments, the macroparticles according to the present teachings may be flakes having an aspect ratio (or average aspect ratio) within a range of 2:1 to 500:1, 4:1 to 500:1, 8:1 to 500:1, 20:1 to 500:1, 20:1 to 300:1, 20:1 to 250:1, 20:1 to 200:1, or 20:1 to 100:1.

FIGS. 8A and 8B schematically illustrate matrix flakes 705, embedding pigment particles 701. The above-mentioned characteristic measures are shown, on a cross sectional view (FIG. 8A) and on a top view (FIG. 8B) of a schematic flake. The exemplary flakes shown in these figures illustrate the particular case of tentacular flakes having “core” bodies from which more narrow appendices extend.

Additionally, the presence of one or more tentacles on a flake matrix element, as illustrated in the top view provided in FIG. 8B, can facilitate the redispersion of matrix elements produced by the present method. As such, matrix elements may be sufficiently large to tend to separate from their carrier over time, the presence of extensions of the elements may serve to increase steric hindrance between neighbouring macroparticles, so that any assembly of such elements would be loose and easily dispersible into individual elements upon mild agitation.

It is believed that the shape of the matrix macroparticles may affect the need to include further dispersants or increased amount of dispersant, at same or different steps. The Applicant found that matrix macroparticles having a tentacular flake shape loosely flocculate, so that advantageously no further dispersants are needed in compositions consisting of such matrix elements.

While the method of preparing the macroparticles of swelled biodegradable polymer embedding dispersed pigment particles could end after the formation of the macroparticles upon completion of a milling step, as represented by S104 in FIG. 9, in some embodiments, the method for preparing the composition may include additional steps.

Such steps can include, for illustration, replacing at least a part of the swelling agent(s) and/or the carrier oil having optionally served for the dispersion of the pigment particles (or mixtures of any of the foregoing liquids) by a different fluid (e.g., a third liquid, a third oil, a volatile liquid, etc.). Such optional step is represented by S105 in FIG. 9.

In one such case, matrix elements having a first softening temperature when associated with the original swelling agent(s) and/or pigment carrier oil(s) can be tailored to have a different second softening point following such partial replacement. Preferably, the second softening point is greater than the first softening point, and optionally greater than 50° C. For such purpose, the swelling agent(s) to be replaced need be sufficiently volatile and the replacing oil can be selected to fulfil at least one of the following conditions: a) it cannot swell the thermoplastic biodegradable polymer under consideration (e.g., resulting in a gain weight of less than 1 wt. %); and b) it does not act as a plasticizer towards the thermoplastic biodegradable polymer under consideration (e.g., it does not lower the softening point of the polymer). Such at least partial replacement can be performed by evaporation of the volatile liquid embedded in the matrix elements (e.g., under vacuum), resulting in relatively dried macroparticles. At least part of the weight loss resulting from the partial elimination of the original liquid(s) can be compensated by addition of the third liquid, which may additionally serve to redisperse the relatively dried matrix elements having consequently a higher softening point.

However, this is only one example of formulation of the macroparticles of swelled biodegradable thermoplastic polymer and any other adaptations of the method to prepare a desired composition are encompassed. For illustration, if the presence of additives is desired, they can be added at any suitable step during the process. The macroparticles obtained upon completion of S104 (or any other subsequent step such as S105) can be separated and transferred as raw material for the preparation of a commercial end-product.

In some embodiments, the end-product composition is a cosmetic composition, manufactured and formulated for application to skin, hair or nails of a human subject. In some embodiments, the end-product composition is manufactured and formulated as a composition for application to a surface of an inanimate object.

In particular embodiments, the cosmetic composition comprises nanoparticles of a UV-protective agent, and the cosmetic composition is a UV-protective composition, such as sunscreen. In other embodiments, the cosmetic composition comprises color-imparting pigment particles embedded in the biodegradable polymeric matrix, and it is formulated as a skin-care or hair-care composition.

The composition of the present invention is preferably in the form of a topical composition. The composition can be in any form suitable to skin-care products, such as facial-care and body-care products (e.g., facial cream, facial mask, body lotion, sunscreens and sunblocks), make-up products (e.g., lipstick, mascara, rouge, foundation, blush, eyeliner, lip liner, lip gloss, lip balm, facial or body powder and artificial tanning lotion), hand-care products and/or foot-care products. Such skin-care products can be applied to the skin of a subject by any conventional method and/or for any duration of time that need not be detailed herein.

The composition of the present invention may also be in the form of a hair-care product, such as a shampoo, a conditioner and a hair mask. Such hair-care products can be applied to the hair of a subject by any conventional method and/or for any duration of time that need not be detailed herein.

In some embodiments of the methods disclosed herein, the subject who may benefit from the compositions prepared thereby is a human subject. In alternative embodiments of a use of the composition, the subject is a non-human animal.

In some embodiments of the use of the composition, the target surface is a surface of an inanimate object, such as, for example, an object, or a material. In some such embodiments, the composition is in the form of a coating, including liquid coatings, such as a varnish, a lacquer or an emulsion, and non-liquid coatings, such as a paste, a gel, or a mousse. Though UV-protective compositions applicable to the surfaces of inanimate objects are herein referred to as “coatings”, it will be readily understood that such compositions may also permeate, impregnate or be otherwise embedded at least to some extent within the surfaces of the objects being protected. Such coating products can be applied to the surface of an inanimate object by any conventional method that need not be detailed herein. Even when used to cover or coat inanimate objects, compositions comprising pigment particles embedded in biodegradable polymers provide an advantage, being more environmentally friendly both for the manufacture and handling of these compositions, as well as for their maintenance on those surfaces.

In some embodiments, protecting against ultraviolet radiation, as afforded by the present compositions (e.g., as may be obtained by the present methods) comprises protecting against a harmful effect of ultraviolet A radiation and ultraviolet B radiation.

As used herein, the term “ultraviolet-absorbing agent” refers to an agent providing at least 50% absorption of ultraviolet light in the wavelength range from 290 nm to 400 nm when present in a composition at up to 50% (w/w) of the total composition.

Inorganic pigments possess different absorbance capabilities. Some, like titanium dioxide, may absorb mainly in narrow ranges, e.g., generally in the UVB range (280-315 nm), while others, like zinc oxide, may demonstrate broader absorbance, e.g., in both UVA and UVB ranges. Mixtures of such pigment may be useful in covering the whole UV range, thus providing for broader protection against UV radiation.

When the pigment particles are nanoparticles of a UV-absorbing agent, having a maximal particle size not exceeding 250 nm (e.g., having a D_{V}100 of less than 250 nm, a D_{V}90 of less than 120 nm and a D_{V}50 of less than 50 nm), such size may significantly reduce the maximum wavelength of light, including UV light, which can effectively be absorbed by the particles (causing a blue-shift in the absorption spectrum). UV-protective compositions according to the present teachings comprising doped or undoped crystals of solid inorganic material milled to nanoparticle size may in some embodiments still provide substantial absorption of UV radiation of wavelength from 280 nm (or even shorter wavelength) up to about 400 nm, thus providing broad-spectrum protection against both UVA and UVB radiation, even in the absence of additional ultraviolet-absorbing agents.

As used herein, the term “broad-spectrum UV absorption” with regard to an ultraviolet-absorbing agent refers to the situation in which the area under the curve (AUC) formed by the UV-absorption of the agent as a function of wavelength in the range of 280 nm to 400 nm (AUC₂₈₀₋₄₀₀) is at least 75% of the AUC formed by the same agent at the same concentration in the range of 280 nm to 700 nm (AUC₂₈₀₋₇₀₀). Similarly, where noted as such herein, the terms “broader-spectrum UV absorption” and “broadest spectrum UV absorption” with respect to a UV-absorbing agent refer respectively to the situation in which the area under the curve (AUC) formed by the absorption of the agent as a function of wavelength in the range of 280 nm to 400 nm (AUC₂₈₀₋₄₀₀) is at least 85% or 95% of the AUC formed by the same agent at the same concentration in the range of 280 nm to 700 nm (AUC₂₈₀₋₇₀₀).

In some embodiments, the area under the curve (AUC) formed by the UV-absorption of the composition as a function of wavelength in the range of 280 nm to 400 nm (AUC₂₈₀₋₄₀₀) is at least 75%, at least 85% or at least 95% of the AUC formed by the same composition in the range of 280 nm to 700 nm (AUC₂₈₀₋₇₀₀).

As used herein, the term “critical wavelength” is defined as the wavelength at which the area under the absorbance spectrum from 290 nm is 90% of the integral of the absorbance spectrum from 290 nm to 400 nm. This term is typically used in connection with UV-absorbing agents, which alone or in combination, provide a UV-protective effect over a relatively broad part of the UV range.

In some embodiments, the composition has a critical wavelength of at least 366 nm, such as 367 nm, 368 nm, 369 nm, 370 nm, 371 nm, 372 nm, 373 nm, 374 nm, 375 nm, 376 nm, 377 nm, 378 nm, 379 nm, 380 nm, 381 nm, 382 nm, 383 nm, 384 nm, 385 nm, 386 nm, 387 nm, 388 nm, 389 nm, 390 nm, 391 nm, 392 nm, or greater than 392 nm.

The AUC in various ranges or the critical wavelengths for a given composition are preferably determined at a concentration of pigment at which the absorbance is below saturation, and has for instance a value not exceeding 5.0. As the extent of absorbance may depend on the identity of the pigment particles, the concentration of pigment particles that may provide for absorbance curves not exceeding an upper value of 5.0 may also vary. In some embodiments, such AUC and critical wavelength values are determined for a pigment concentration of 0.001 wt. %, 0.005 wt. %, 0.01 wt. %, or 0.05 wt. %, by total weight of the sample being tested.

In some embodiments of the composition, use or method disclosed herein, the composition contains less than 5 wt. % organic UV-absorbing agents. In some embodiments the composition contains less than 4 wt. %, 3 wt. %, 2 wt. % or 1 wt. % organic UV-absorbing agents.

In some embodiments the composition is largely free of organic ultraviolet-absorbing agents, i.e. the composition contains less than 0.5 wt. % organic UV-absorbing agents. In some embodiments the composition is mostly free of organic UV-absorbing agents, i.e. the composition contains less than 0.1 wt. % organic UV-absorbing agents. In some embodiments the composition is principally free of organic ultraviolet-absorbing agents, i.e. the composition contains less than 0.05 wt. % organic UV-absorbing agents. In some embodiments the composition is fundamentally free of organic UV-absorbing agents, i.e. the composition contains less than 0.01 wt. % organic UV absorbing agents. In some embodiments of the composition, use or method disclosed herein, the composition is generally devoid of organic ultraviolet-absorbing agents, considerably devoid of organic ultraviolet-absorbing agents, significantly devoid of organic ultraviolet-absorbing agents, substantially devoid of organic ultraviolet-absorbing agents, essentially devoid of organic ultraviolet-absorbing agents, substantively devoid of organic ultraviolet-absorbing agents or devoid of organic ultraviolet-absorbing agents.

As used herein, the terms “generally devoid of an organic UV-absorbing agent”, “considerably devoid of an organic UV-absorbing agent”, “significantly devoid of an organic UV-absorbing agent”, “substantially devoid of an organic UV-absorbing agent”, “essentially devoid of an organic UV-absorbing agent”, “substantively devoid of an organic UV-absorbing agent” and “devoid of an organic UV-absorbing agent” refer respectively to a composition in which a UV-absorbing organic material, if any, is present in the composition at a concentration which provides absorption of not more than 20%, not more than 15%, not more than 10%, not more than 5%, not more than 2%, not more than 1% or not more than 0.5% of ultraviolet light in the wavelength range from 290 nm to 400 nm.

In some embodiments of the composition, use or method disclosed herein, the composition contains less than 10 wt. % of additional UV-absorbing agents. In some embodiments the composition contains less than 5 wt. %, less than 4 wt. %, less than 3 wt. %, less than 2 wt. % or less than 1 wt. % of additional UV-absorbing agents. In some embodiments the composition is largely free of additional ultraviolet-absorbing agents, i.e. the composition contains less than 0.5 wt. % additional UV-absorbing agents. In some embodiments the composition is mostly free of additional UV-absorbing agents, i.e. the composition contains less than 0.1 wt. % additional UV-absorbing agents. In some embodiments the composition is principally free of additional ultraviolet-absorbing agents, i.e. the composition contains less than 0.05 wt. % additional UV-absorbing agents. In some embodiments the composition is fundamentally free of additional UV-absorbing agents, i.e. the composition contains less than 0.01 wt. % additional UV absorbing agents. In some embodiments of the composition, use or method disclosed herein, the composition is generally devoid of additional ultraviolet-absorbing agents, considerably devoid of additional ultraviolet-absorbing agents, significantly devoid of additional ultraviolet-absorbing agents, substantially devoid of additional ultraviolet-absorbing agents, essentially additional of organic ultraviolet-absorbing agents, substantively additional of organic ultraviolet-absorbing agents or devoid of additional ultraviolet-absorbing agents.

In some embodiments of the composition, use or method disclosed herein, the pigment particles, including in particular the nanoparticles of inorganic UV-absorbing agent, are the sole pigment particles providing for the sought effect. In other embodiments, different types of compounds and pigment particles can be used to achieve a desired effect. For instance, a composition intended to provide at least a coloring effect may include a mixture of pigment particles (each type of pigments providing for a specific shade) or a mixture of pigments and dies.

In embodiments wherein the composition is intended to provide at least a UV-protective effect, the composition may comprise more than one inorganic UV-absorbing agent, wherein each such agent provides protection against different ranges of the UV radiation. For instance, the composition may comprise three UV-absorbing agents, one absorbing radiation in the UVA range, another absorbing in the UVB range and yet another absorbing in the UVC range.

As used herein, the term “generally devoid of an additional ultraviolet-absorbing agent”, “considerably devoid of an additional ultraviolet-absorbing agent”, “significantly devoid of an additional ultraviolet-absorbing agent”, “substantially devoid of an additional ultraviolet-absorbing agent”, “essentially devoid of an additional ultraviolet-absorbing agent”, “substantively devoid of an additional ultraviolet-absorbing agent” and “devoid of an additional ultraviolet-absorbing agent” refer respectively to a composition which is devoid of any UV-absorbing material other than that specifically disclosed as being present in the composition at a concentration, which, if included in the composition, provides absorption of not more than 20%, not more than 15%, not more than 10%, not more than 5%, not more than 2%, not more than 1% or not more than 0.5% of ultraviolet light in the wavelength range from 290 nm to 400 nm.

Unless otherwise defined, all technical and scientific terms used herein have the same meaning as commonly understood by one of ordinary skill in the art to which the disclosure pertains. In case of conflict, the specification, including definitions, will take precedence.

As used herein, the terms “comprising”, “including”, “having” and grammatical variants thereof are to be taken as specifying the stated features, integers, steps or components, but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise. For instance, a thermoplastic biodegradable polymer can include a mixture of polymers, an oil can include a mixture of oils, a UV-protective agent can include a mixture of UV-protective agents as herein disclosed and so on.

In the discussion, unless otherwise stated, adjectives such as “substantially” and “about” that modify a condition or relationship characteristic of a feature or features of an embodiment of the present technology, are to be understood to mean that the condition or characteristic is defined within tolerances that are acceptable for operation of the embodiment for an application for which it is intended. In particular, when a numerical value is preceded by the term “about”, the term “about” is intended to indicate +/−10%, or +1-5%, or +/−2% of the mentioned value.

Additional objects, features and advantages of the disclosure will be set forth in the detailed description which follows, and in part will be readily apparent to those skilled in the art from the description or recognized by practicing the disclosure as described in the written description and claims hereof, as well as the appended drawings. Various features and sub-combinations of embodiments of the disclosure may be employed without reference to other features and sub-combinations.

It is to be understood that both the foregoing general description and the following detailed description, including the materials, methods and examples, are merely exemplary of the disclosure, and are intended to provide an overview or framework to understanding the nature and character of the disclosure as it is claimed, and are not intended to be necessarily limiting.

### EXAMPLES

### Materials

The materials used in the following examples are listed in Table 1 below. The reported information was retrieved the product data sheets provided by the respective suppliers. Unless otherwise stated, all materials were purchased at highest available purity level.

TABLE 1

| Type | Chemical Name | Product Name | Supplier | CAS No. |
|---|---|---|---|---|
| Pigments | Micro titanium | MT-100TV | Tayca, Japan | 13463-67-7 |
| | dioxide | (15 nm) | | |
| | Micro titanium | MT-700Z | | |
| | dioxide | (80 nm) | | |
| | Micro zinc oxide | MZY-505EX | | 1314-13-2 |
| | | (25 nm) | | |
| Biodegradable | Polylactic acid | Ecosoft ® 608XF | Micro | 9051-89-2 |
| thermoplastic | (PLA) | | Powders, USA | |
| polymers | Polycaprolactone | Polycaprolactone, | Sigma | 24980-41-4 |
| | (PCL) | average MW: ~14,000 | Aldrich ®, USA | |
| | Polyhydroxy- | Enmat Y3000 | TianAn | 26744-04-7 |
| | butyrate (PHB) | | Biologic | |
| | Poly(3- | Enmat Y1000 | Materials, | 80181-31-3 |
| | hydroxybutyrate- | | China | |
| | co-3- | | | |
| | hydroxyvalerate) | | | |
| | (PHBV) | | | |
| | Poly(butylene | Poly(butylene | Dideu, China | 25777-14-4 |
| | succinate) (PBS) | succinate) | | |
| | Poly(butylene | Poly(butylene | Dideu, China | 67423-06-7 |
| | succinate-co- | succinate-co- | | |
| | adipate) (PBSA) | adipate) | | |
| | Dewaxed shellac | Dewaxed shellac | Worlée, | 9000-59-3 |
| | | type 4621 | Germany | |
| | Gum rosin | Gum rosin | Guangxi | 8050-09-7 |
| | | | Wuzhou | |
| | | | Foreign Trade, | |
| | | | China | |
| Swelling agent/ | Dibutyl adipate | Cetiol ® B | BASF, | 105-99-7 |
| oil carrier | | | Germany | |
| | Benzyl alcohol | Benzyl alcohol | Sharon | 100-51-6 |
| | | | Laboratories, | |
| | | | Israel | |
| | Butyl lactate | Butyl lactate | Tokyo | 138-22-7 |
| | | | Chemical | |
| | | | Industry Co. | |
| | | | (TCI), Japan | |
| | Cinnamon bark | Cinnamon bark | Nature In | 8015-91-6 |
| | oil | oil (CBO) | Bottle, India | |
| | Caprylyl | Cetiol ® RLF | BASF, | 2306-88-9 |
| | caprylate/caprate | | Germany | 2306-92-5 |
| | Lauryl lactate | Dermol LL | Alzo | 6283-92-7 |
| | | | International, | |
| | | | USA | |
| | Triethyl citrate | Citrofol ® AI | Jungbunzlauer | 77-93-0 |
| | Acetyltriethyl | Citrofol ® AII | International, | 77-89-4 |
| | citrate | | Switzerland | |
| | Glyceryl | Glyceryl | Sigma | 538-23-8 |
| | trioctanoate | trioctanoate | Aldrich ®, USA | |
| Dispersant | Polyhydroxy- | Pelemol ® PHS-8 | Phoenix | 27924-99-8 |
| | stearic acid | | Chemicals, | |
| | | | USA | |
| | | Dispersun | Innospec | 58128-22-6 |
| | | DSP-OL100 | Performance | |
| | | Dispersun | Chemicals, | |
| | | DSP-OL300 | USA | |
| Volatile liquid | Isopropyl alcohol | IPA | Sigma | 67-63-0 |
| | | | Aldrich ®, USA | |

### Equipment

Grinding mill for pigment dispersion: Dyno®-Mill Research Lab (WAB Group, Switzerland)
Grinding mill for macroparticles preparation: HD-01 Attritor (Union Process, USA)
Zirconium oxide/yttrium stabilized 0.1-0.2 mm and 2 mm ceramic beads (SiLibeads, type ZY-E, Sigmund Linder, Germany)
Particle Size Analyser (Dynamic Light Scattering): Zen 3600 Zetasizer (Malvern Instruments, United Kingdom)
Macroparticle Size Analyser (Light Scattering, laser diffraction): Mastersizer 2000 (Malvern Instruments, United Kingdom)
Optical microscope: Olympus BX-51 (Olympus, Japan)
Oven: Carbolite PF 200 (MRC, Israel)
Hot plate: MH-4 (MRC Lab, Israel)
Disperser: T25 digital Ultra Turrax® (IKA Works, Germany)
Spectrophotometer: Cary 500 UV-Vis (Agilent, USA)
High Resolution Scanning Electron Microscope: Crossbeam 340 (Zeiss, Germany)
Vortex: Vortex-Genie 2 (Scientific Industries, USA)
Centrifuge: Centrifuge Z 383 (Hermle Labortechnik, Germany)
High Resolution Scanning Electron Microscope: Magellan XHR 400L FE-SEM (Nanolab Technologies, USA)
Double Planetary Mixer: DPM-1PT (Charles Ross & Son, USA).

### Example 1: Preparation of Dispersions of Particles of UV-Protective Agents

In a 500 ml glass container, 192.5 g of titanium dioxide (TiO₂, having primary particles of about 15 nm, but supplied as MT-100TV with secondary aggregates in the micron range) were combined with 19.25 g of Pelemol® PHS-8 dispersant and mixed by hand. 138.25 g of an oil carrier (Cetiol® B) were then added as a liquid carrier, and mixed by hand until a homogeneous mixture was obtained, the mixture having a TiO₂/dispersant weight by weight (w/w) ratio of 10:1, i.e. 55 wt. % TiO₂ and 5.5 wt. % Pelemol® PHS-8 dispersant, by weight of the composition.

The mixture was placed in a Dyno®-Mill Research Lab grinding mill, loaded with 55 ml of 0.1-0.2 mm zirconium oxide/yttrium stabilized ceramic beads, and the grinding mill was activated at 5,400 rounds per minute (rpm) for 2 hours (hrs) at 55° C. to obtain a dispersion of pigment particles in a desired size range.

The composition of the dispersed nanoparticles is reported in Table 2 below as Ti-55, according to the 55% wt. % amount of TiO₂ in the dispersion. Additional dispersions of pigments having a primary particle size in the low nanometer range (e.g., 15 and 25 nm) were prepared according to similar procedures, each containing different components in different amounts, being dispersed under different conditions, all of which are specified in the table. The values reported in the table correspond to the concentration of each ingredient in weight percent (wt. %) by total weight of the dispersion. For reference, the zinc oxide pigments being dispersed have primary particles of about 25 nm.

After the dispersing period, samples of the dispersed nanoparticles were further diluted in their respective oil carrier to form suspensions having a solid inorganic concentration of about 0.1 wt. % and the hydrodynamic diameter of the nanoparticles was determined by DLS in the diluted sample.

The particle size distribution of the nanoparticles prepared as herein described, namely the maximum hydrodynamic diameters for particular percentages of the population, are provided in Table 2 below, in terms of percent of volume of particles.

TABLE 2

| | | Ti-40 | Ti-40 | Ti-40 | |
|---|---|---|---|---|---|
| Component | Ti-55 | (GtO) | (RLF) | (GtO-RLF) | Zn-40 |
| TiO₂ (15 nm) | 55 | 40 | 40 | 40 | |
| ZnO (25 nm) | | | | | 40 |
| Pelemol ® PHS-8 | 5.5 | 4 | 4 | 4 | 4 |
| Cetiol ® B | 39.5 | | | | |
| Glyceryl trioctanoate | | 56 | | 42 | |
| (GtO) | | | | | |
| Cetiol ® RLF | | | 56 | 14 | 56 |
| Dispersing | 2 hrs, 55° C. | 4.5 hrs, 57° C. | 3 hrs, 48° C. | 4 hrs, 57° C. | 7 hrs, 42° C. |
| conditions | 5,400 rpm | 5,000 rpm | 5,000 rpm | 5,000 rpm | 5,000 rpm |
| Dv10 [nm] | 28.5 | 26.1 | 24.4 | 23.3 | 29.4 |
| Dv50 [nm] | 44.5 | 38.1 | 35.1 | 33.8 | 41.7 |
| Dv90 [nm] | 84.5 | 62.3 | 55.3 | 53.6 | 65.0 |
| Polydispersity | 0.15 | 0.17 | 0.19 | 0.20 | 0.10 |
| index (PDI) | | | | | |

As can be seen in Table 2, the present method is suitable to disperse the titanium dioxide and zinc oxide stock into secondary particles well in the nano range, even the maximum diameter of the population (e.g., D_{V}90) not exceeding 100 nm and the lower end of the population (e.g., D_{V}10) being commensurate with the size of the primary particles of the pigments. Moreover, it can be noted that these nanoparticles have a relatively narrow distribution, their respective (D90−D10)/D50 being at most 1.26, hence all well below 2. The polydispersity index (PDI) of the population of dispersed nanoparticles was at most 0.2.

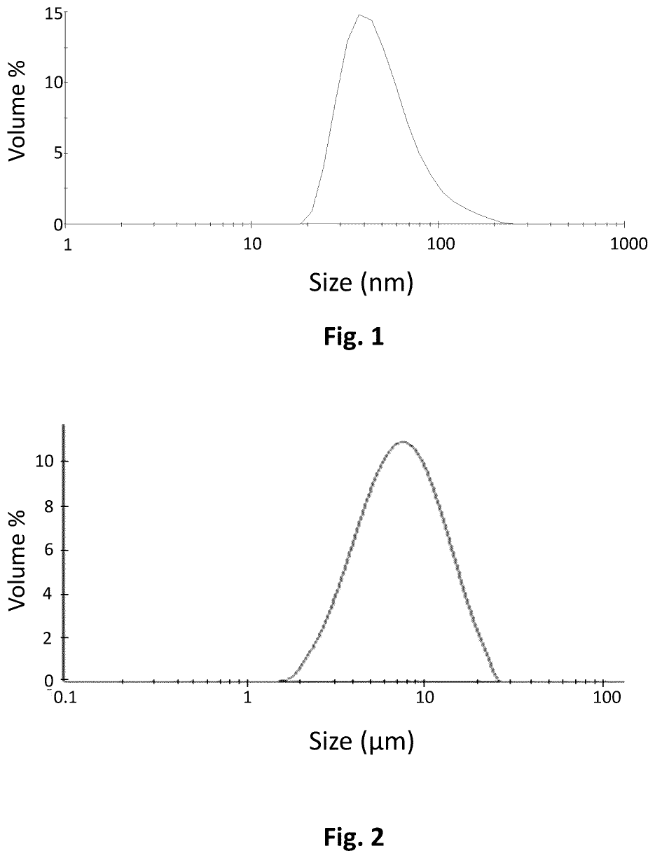

Representative results of a sample of the Ti-55 composition, showing the percentage (per volume) of titanium dioxide particles having hydrodynamic diameters in the range of 1-1,000 nm are presented in FIG. 1, the size of the nanoparticles being mainly confined in the 20 nm to 250 nm range.

Additional dispersions were similarly prepared using TiO₂ pigment and Dermol LL, Citrofol® AI or Citrofol® AII as oil carriers instead of Cetiol® B. Other dispersants, mainly Dispersun DSP-OL100 and Dispersun DSP-OL300, were also used instead of Pelemol® PHS-8. The various oil carriers and dispersants provided PSD results similar to the ones obtained when using Cetiol® B and Pelemol® PHS-8.

Another dispersion was prepared in absence of a dedicated dispersant. 1 wt. % of TiO₂, as pigment having primary particles of about 15 nm, were dispersed in Pelemol® 256, as oil carrier, for 8.5 hours at 35° C. and at 5,000 rpm. The obtained dispersion demonstrated a reduction in the PSD of the dispersed particles as compared to their size distribution before dispersing. The PSD of the dispersion obtained in absence of a dispersant supports its suitability for use in the preparation of the present compositions, the dispersion having a D_{V}98 of less than 700 nm and a PDI of 0.18.

Finally, a dispersion or microparticles was prepared, by combining 175 g of titanium dioxide (TiO₂, having primary particles of about 80 nm, and supplied as MT-700Z with secondary aggregates in the micron range) with 17.5 g of Pelemol® PHS-8 as dispersant and 157.5 g of Cetiol® B as an oil carrier, as described above. The mixture was placed in an attritor with 770 ml of 2 mm zirconium oxide/yttrium stabilized ceramic beads, and dispersed at a temperature of 20° C. (maintained using a jacket) at 700 rpm for 2 hours.

The resulting microparticles dispersion (containing 50 wt. % of TiO₂ microparticles and accordingly referred to as Ti-50) demonstrated a particle size distribution with a D_{V}10 of 168 nm, a D_{V}50 of 275 nm, and a D_{V}90 of 410 nm. The size distribution was narrow, the PDI of the population of dispersed microparticles being of 0.14, such PSD parameters supporting the suitability of the dispersed microparticles of pigments for use, e.g., in preparing colored compositions.

### Example 2: Selection of Thermoplastic Swellable Biodegradable Polymers

In order to assess the suitability of thermoplastic biodegradable polymers to embed dispersed pigment particles, a screen was performed to assess their compatibility first with a predetermined swelling agent and secondly with a predetermined dispersant. For this purpose, a known amount of a biodegradable polymer (generally 2 g in the form of powder or granules, as provided by suppliers) was weighted and placed in a 20 ml sealable glass vial, then combined with the material with which assessment of compatibility was sought.

The biodegradable polymers so tested included poly(butylene succinate) (PBS), poly(butylene succinate-co-adipate) (PBSA), polyhydroxybutyrate (PHB), poly(3-hydroxy-butyrate-co-3-hydroxyvalerate) (PHBV), polycaprolactone (PCL), polylactic acid (PLA), and mixtures thereof, such as PLA: PHB (at a 1:1 weight ratio) and PLA:PCL (at a 9:1 weight ratio).

When swelling agents were screened, 8 g of the liquid being tested were added to the polymer being considered, the vial was sealed, and its contents mixed by vortex. The sealed vials were then incubated for 24 hours in an oven set at 50° C. If the liquid was totally absorbed by the polymer, forming a continuous paste, it was defined as a swelling agent compatible with the polymer being tested. If the liquid was only partially absorbed, if at all, the polymer, whether or not significantly swollen, was filtered out of the remaining liquid, rinsed with a volatile liquid and dried, before being weighted. A liquid providing for a weight gain of at least 5% for the polymer, was also deemed a compatible swelling agent. Conversely, a polymer which was swollen by such suitable swelling agents was deemed a swellable polymer and its compatibility with additional materials, such as dispersants as below described, was further investigated.

The swelling agents so tested, which successfully swelled at least one of the afore-said polymers but typically all, included 2-ethylhexyl lactate, acetyl tributyl citrate, acetyl triethyl hexyl citrate, acetyl triethyl citrate, allyl hexanoate, benzyl alcohol, benzyl benzoate, butyl butyryl lactate, butyl lactate, cinnamon bark oil, dibutyl adipate, dibutyl maleate, dibutyl sebacate, diethyl succinate, diethylene glycol diethyl ether, dimethyl glutarate, dimethyl isosorbide, dimethyl maleate, dimethyl methylglutarate, ethyl lactate, gamma decalactone, lactic acid isoamyl ester, lauryl lactate, 1-menthyl lactate, menthalactone, n-pentyl benzoate, PEG-6 caprylic/capric glycerides, triacetin, triethyl citrate and undecanoic lactone.

When dispersants were screened, they were added to the swellable polymer being considered in one or more arbitrary amounts deemed suitable to later disperse pigment particles. Usually a few grams, not exceeding 2 g, were added to each swellable polymer being tested for compatibility. The vials were sealed and placed on a hot plate at a temperature suitable to melt the polymer together with the liquid dispersant. The melted polymer mixture so obtained was manually stirred with a metal spatula until forming a homogeneous melt, and one drop was placed on a microscope slide, covered with a glass cover slide and cooled to room temperature (circa. 23° C.). The appearance of the polymer-dispersant cooled melts was analysed using an optical microscope.

The results obtained with Pelemol® PHS-8 as dispersant as tested with a variety of swellable polymers are summarized in Table 3 below.

TABLE 3

| | Melting | | |
|---|---|---|---|
| Polymer | point (Tm) | | Result |
| Poly(butylene succinate) (PBS) | 114° | C. | Clear |
| Poly(butylene succinate-co-adipate) (PBSA) | 111° | C. | Clear |
| Polycaprolactone (PCL) | ~60° | C. | Hazy |
| Polyhydroxybutyrate (PHB) | 175-180° | C. | Clear |
| poly(3-hydroxybutyrate-co-3- | 153° | C. | Clear |
| hydroxyvalerate) (PHBV) | | | |
| Polylactic acid (PLA) | 170-180° | C. | Clear |

| | | |
|---|---|---|
| PLA + PHB | NA | Clear |
| PLA + PCL | NA | Clear |

As can be seen, even when a polymer alone is less compatible with a particular dispersant, as can be estimated from the hazy appearance of the cooled melt observed with PCL, this can be countered by mixing the less compatible polymer with a more compatible one. Mixing 10 wt. % of PCL with 90 wt. % of PLA produced a clear melt, supporting the compatibility of the polymers with the dispersant. Without wishing to be bound by a particular theory, a hazy appearance is believed to indicate an incompatible mixture, such as resulting in a bi-phasic emulsion.

### Example 3: Preparation of Swelled Biodegradable Polymer Matrix

20.4 g of polylactic acid (PLA) were placed in a 800 ml glass container. 379.6 g of swelling agent (Cetiol® B) were added, and the container was sealed and placed in an oven, where it was maintained for 24 hours at 120° C. allowing the polymer to swell. After allowing the swelled biodegradable polymer (SBP) to cool down for 15 minutes at room temperature (RT), it was sheared together with the excess liquid not absorbed in the process. Shearing was performed at RT with a high-speed disperser for 20 minutes at 2,420 rpm, during which the temperature of the composition was allowed to naturally decrease. Such mixing achieved two goals, first the formation of a homogeneous paste of swelled biodegradable polymer, at least as long as the SBP remained sufficiently soft, and secondly the preliminary comminution of the paste into individual morsels, at least as soon as the SBP turned sufficiently relatively solid as the temperature dropped. The morsels so prepared were expected to facilitate a future embedment of pigment particles and the further size reduction to macroparticles. A swelled biodegradable polymer composition, having a polymer concentration of 5.1 wt. %, by weight of the composition, was obtained and was accordingly named SBP5.1-PLA.

The swelling process was repeated with different swellable biodegradable polymers and swelling agents and modified accordingly. The initial mixtures of the polymers and swelling agents were placed in the oven for periods of 10-24 hours at a temperature of 80-120° C., the swelling temperature taking into account the respective melting temperatures of each polymer. The obtained swelled polymer mixtures were sheared at a low shear rate for homogenization and preliminary morselizing as described above, or allowed to cool without any shearing, when the swelled polymer was sufficiently morselized or soft. The morsels of swelled polymer were then milled with pigment particles as illustrated in Example 4. The various SBP compositions so obtained were typically allowed to cool for a duration of time sufficient to reach a temperature of about 50° C., but were also subsequently used at lower temperatures and having reached RT.

The SBP compositions so prepared are reported in Tables 4A and 4B, each composition containing different materials in different amounts, as specified in the table. CBO stands for cinnamon bark oil, and the polymer mixes were prepared with 50:50 g of each for PLA:PHB and with 90:10 g of each for the PLA:PCB. The values reported in the table correspond to the concentration of each component in weight percent (wt. %) by total weight of the SBP composition.

TABLE 4A

| | SBP | SBP | SBP | SBP | SBP | SBP | SBP |
|---|---|---|---|---|---|---|---|
| | 5.1- | 6.1- | 10.2- | 12.2- | 33.7- | 40- | 30- |
| Material | PLA | PLA | PLA | PLA | PLA | PHB | PHB |
| PLA | 5.1 | 6.1 | 10.2 | 12.1 | 33.7 | | |
| PHB | | | | | | 40 | 30 |
| Dibutyl adipate | 94.9 | 93.9 | 89.8 | 87.8 | 66.3 | | |
| Benzyl alcohol | | | | | | 60 | 70 |

TABLE 4B

| | SBP | SBP | | | | SBP 20- |
|---|---|---|---|---|---|---|
| | 30- | 30- | SBP 30- | SBP 30- | SBP 20- | gum |
| Material | PHBV | PBSA | PLA:PHB | PLA:PCL | shellac | rosin |
| PHBV | 30 | | | | | |
| PBSA | | 30 | | | | |
| PLA:PHB | | | 30 | | | |
| PLA:PCL | | | | 30 | | |
| Shellac | | | | | 17 | |
| Gum rosin | | | | | | 92 |
| Butyl | 70 | | | | | |
| lactate | | | | | | |
| CBO | | 70 | | | | |
| Lauryl | | | 70 | 70 | | |
| lactate | | | | | | |
| Dibutyl | | | | | 83 | 8 |
| adipate | | | | | | |

### Example 4: Preparation of Compositions Comprising Macroparticles of Swelled Biodegradable Polymer Matrix and Nanoparticles of a UV-Protective Agent

6.36 g of the Ti-55 dispersion of TiO₂ nanoparticles, prepared as described in Example 1, were placed in a 800 ml glass container. 343.64 g of swollen polymer composition SBP5.1-PLA, prepared as described in Example 3, were added and mixed manually to give a final weight of 350 g.

The obtained mixture was placed in a zirconia pot of an attritor, with 770 ml of 2 mm zirconium oxide/yttrium stabilized ceramic beads. The temperature of the pot was maintained at 50° C. by a jacket while the attritor was set to mill the contents of the pot at 750 rpm for 5 hours, resulting in a composition according to the present teachings comprising inorganic nanoparticles of a UV-protective agent dispersed and embedded in macroparticles of the swelled biodegradable polymer matrix.

This composition is reported in Table 5 as Comp. 1. Additional compositions were prepared according to the above procedure, each composition containing different amounts of each component, as specified in the table. The values reported in the table correspond to the concentration of each ingredient in weight percent (wt. %) by weight of the total composition.

TABLE 5

| Component | Comp. 1 | Comp. 2 | Comp. 3 | Comp. 4 | Comp. 5 |
|---|---|---|---|---|---|
| Pigment dispersion | Ti-55 | Ti-55 | Ti-55 | Ti-55 | Ti-55 |
| Swollen polymer | SBP | SBP | SBP | SBP | SBP |
| matrix | 5.1-PLA | 6.1-PLA | 10.2-PLA | 12.2-PLA | 33.7-PLA |
| TiO₂ | 1 | 10 | 1 | 10 | 6 |
| Pelemol ® PHS-8 | 0.1 | 1 | 0.1 | 1 | 0.6 |
| PLA | 5 | 5 | 10 | 10 | 30 |
| Cetiol ® B | 93.9 | 84 | 88.9 | 79.1 | 63.4 |
| Pigment:polymer | 1:5 | 2:1 | 1:10 | 1:1 | 1:5 |
| w/w ratio | | | | | |

Hydrodynamic diameters of the resulting macroparticles were determined, using LS, on a sample of the swelled biodegradable polymer matrix macroparticles containing the titanium dioxide nanoparticles, diluted in Cetiol® B according to the instrument operating instructions. The size distribution of swelled polymer matrix macroparticles having hydrodynamic diameters in the range of 0.1-100 μm are presented in FIG. 2 in percentage (per volume) for an exemplary composition, the size of the macroparticles being mainly confined in the 2 to about 25 μm range.

The particle size distribution of the swelled biodegradable polymer matrix macroparticles prepared as above-described with swollen PLA, namely the maximum hydrodynamic diameters of the matrix elements for particular percentages of the population, are provided for an exemplary composition in Table 6 below, in terms of percent of volume of the macroparticles.

TABLE 6

| 10% | 50% | 90% |
|---|---|---|
| 3.6 μm | 7.3 μm | 14.3 μm |

As can be seen in Table 6, at least 90% of the sampled macroparticles have a hydrodynamic diameter of at most about 14.3 μm, and 50% of the macroparticles population have a hydrodynamic diameter (demonstrated by the predominant peak) of 7.3 μm. Microscopic analysis detailed below determined that the shape of the resulting matrix elements was flake-like. The other compositions prepared using the afore-mentioned swelled PLA SBPs demonstrated similar particle size distributions, the peaks displayed by each population of macroparticles being relatively narrow and typically in a range from about 1 μm to no more than about 30 μm. The sizes assessed by LS were separately confirmed by microscope analysis, which further allowed measuring the thicknesses of the flake-shaped macroparticles. Typically, their thicknesses, depending on composition and milling duration, were in a range between 200 nm and 750 nm.

Similar milling procedures were performed with SBP compositions of additional polymers and different dispersions of pigments. 87.5 g of the Ti-40(GtO) dispersion of TiO₂ nanoparticles, prepared as described in Example 1, were added in a beaker to 87.5 g of swollen polymer composition SBP40-PHB, prepared as described in Example 3, and 175 g of glyceryl trioctanoate (which served as oil carrier for the pigment dispersion). The mixture having a combined weight of 350 g was manually mixed and placed in a zirconia pot of an attritor, loaded with 770 ml of 2 mm zirconium oxide/yttrium stabilized ceramic beads. Alternatively, all ingredients can be directly placed in the attritor and undergo a preliminary mixing at about 150 rpm for a few minutes, before operating the bead mill attritor as below described.

The temperature of the pot was maintained at 50° C. by a jacket while the attritor was set to mill the contents of the pot at 700 rpm for 2 hours. The temperature was then lowered to 30° C. and the milling was continued for 26 more hours. A composition according to the present teachings, comprising inorganic nanoparticles of a UV-protective agent dispersed and embedded in the swelled biodegradable polymer matrix macroparticles, was obtained.

This composition is reported in Table 7 as Comp. 6. Additional compositions were prepared according to similar procedures, each composition containing different components in different amounts, and milled under different milling conditions, as specified in table 7. The values reported in the table correspond to the concentration of each component in weight percent (wt. %) by total weight of the composition. The particle size distributions (as measured by LS) of the swelled biodegradable polymer matrix macroparticles so prepared are reported in terms of percent of volume of particles.

TABLE 7

| Component | Comp. 6 | Comp. 7 | Comp. 8 | Comp. 9 | Comp. 10 | Comp. 11 |
|---|---|---|---|---|---|---|
| Pigment | Ti- | Ti- | Ti- | Ti- | Zn-40 | Ti-40(RLF) |
| dispersion | 40(GtO) | 40(GtO) | 40(RLF) | 40(RLF) | | |
| Swollen | SBP40- | SBP40- | SBP30- | SBP30- | SBP30- | SBP30- |
| polymer | PHB | PHB | PHB | PHBV | PHB | PBSA |
| matrix | | | | | | |
| TiO₂ | 10 | 20 | 20 | 10 | | 10 |
| ZnO | | | | | 10 | |
| Pelemol ® | 1 | 2 | 2 | 1 | 1 | 1 |
| PHS-8 | | | | | | |
| PLA | | | | | | |
| PHB | 10 | 10 | 10 | | 10 | |
| PHBV | | | | 10 | | |
| PBSA | | | | | | 10 |
| Glyceryl | 14 * | 28 * | | | | |
| trioctanoate | 50 *** | 25 *** | | | | |
| Cetiol ® | | | 28 * | 14 * | 14 * | 14 * |
| RLF | | | 16.7 *** | | 41.7 *** | 41.7 *** |
| Benzyl | 15 ** | 15 ** | 23.3 ** | | 23.3 ** | |
| alcohol | | | | | | |
| Butyl | | | | 23.3 ** | | |
| lactate | | | | 41.7 *** | | |
| Cinnamon | | | | | | 23.3 ** |
| bark oil | | | | | | |
| Milling | 2 hrs @ | 2 hrs @ | 2 hrs @ | 2 hrs @ | 2hrs @ | 2 hrs @ |
| conditions | 50° C. then | 50° C. then | 50° C. then | 50° C. then | 50° C. then | 50° C. then |
| | 26 hrs @ | 23 hrs @ | 22.5 hrs @ | 22 hrs @ | 33 hrs @ | 77 hrs @ |
| | 30° C. | 30° C. | 30° C. | 30° C. | 30° C. then | 30° C. |
| | 700 rpm | 700 rpm | 700 rpm | 800 rpm | 18 hrs @ | 700 rpm |
| | | | | | 50° C. | |
| | | | | | 800 rpm | |
| Pigment:polymer | 1:1 | 2:1 | 2:1 | 1:1 | 1:1 | 1:1 |
| w/w ratio | | | | | | |

PSD measurements

| | | | | | | |
|---|---|---|---|---|---|---|
| Dv10 [μm] | 0.2 | 0.1 | 0.6 | 0.1 | 0.1 | 1.0 |
| Dv50 [μm] | 1.6 | 1.6 | 1.7 | 1.0 | 0.5 | 3.4 |
| Dv90 [μm] | 4.5 | 5.1 | 5.0 | 3.0 | 1.5 | 10.8 |

* originating from the nanoparticles dispersions prepared in Example 1
** originating from the swelled biodegradable polymer compositions prepared in Example 3
*** originating from an additional amount of oil being added during milling

As can be seen from the above table, the macroparticles obtained upon completion of milling of the compositions of Table 7 demonstrated particle size distributions typically in a range from about 0.1 μm to no more than about 10 μm and generally even less. The thickness of these flakes was decreased accordingly, so that their aspect ratio remained of platelet type. These results are lower than the PSDs measured for the compositions of Table 5, prepared with shorter duration of milling, and support that the PSD of the macroparticles can be tailored inter alia based on selection of appropriate milling conditions.

### Example 5: Absorbance of Oil-Dispersed Titanium Dioxide Nanoparticles

The dispersion Ti-55, obtained in Example 1, containing Cetiol® B as an oil carrier and Pelemol® PHS-8 as a dispersant, in addition to the nanoparticles of titanium dioxide, was diluted with its carrier to provide TiO₂ concentrations of 0.001 wt. %, 0.003 wt. %, 0.005 wt. % and 0.007 wt. %.

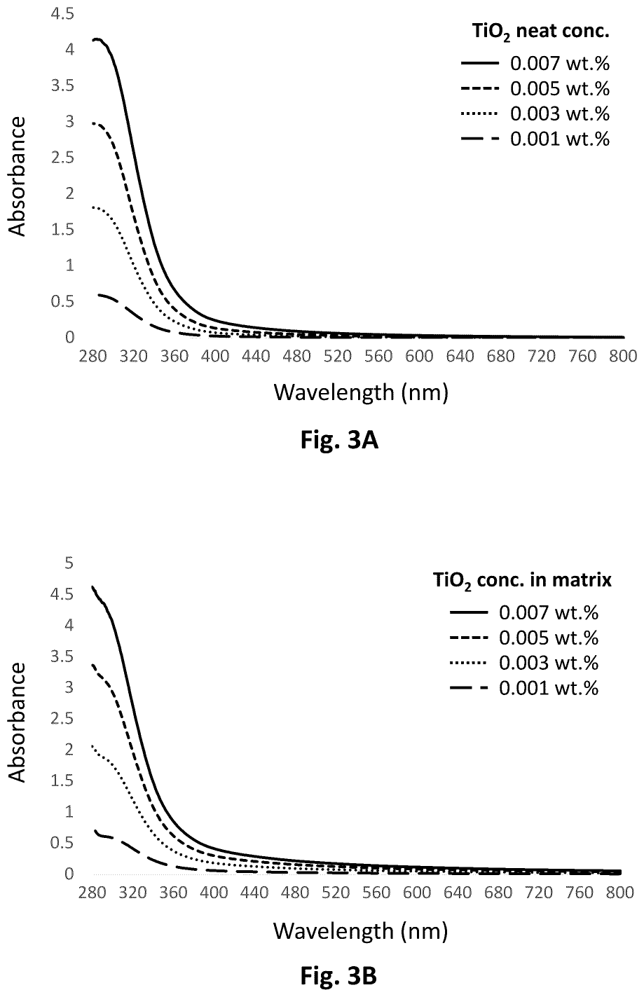

Absorbance of the dispersed titanium dioxide nanoparticles over the wavelength range of 280-800 nm was measured using a spectrophotometer. The dispersion was placed in a 10 mm light pathway quartz cuvette, and the absorbance was measured as compared to a reference cuvette containing only the liquid carrier, Cetiol® B. Results are presented in FIG. 3A. The dispersant absorbance was measured separately and was found to be insignificant.

As seen in FIG. 3A, titanium dioxide nanoparticles dispersed according to the present teachings absorbed light mainly in the UV range, their efficacy dropping below 50% (i.e. displaying an absorbance of less than 0.301) at about 388 nm at its highest tested concentration (0.007 wt. %). Furthermore, the absorbance in this range increases as the concentration of the titanium dioxide nanoparticles increases. The lack of significant absorption in the visible light range confirms that the nanoparticles remained sufficiently dispersed, lacking secondary particles aggregates sufficient large to absorb in the visible range.

### Example 6: Absorbance of Titanium Dioxide Nanoparticles Dispersed and Embedded in Swelled Biodegradable Polymer Matrix Macroparticles

In order to assess the absorbance of the titanium dioxide nanoparticles once embedded in the biodegradable polymer macroparticles, Comp. 3 obtained in Example 4, having a pigment: polymer w/w ratio of 1:10 was used. Comp. 3 was diluted with Cetiol® B to provide compositions comprising TiO₂ nanoparticles at concentrations of 0.001 wt. %, 0.003 wt. %, 0.005 wt. % and 0.007 wt. %.

Absorbance of titanium dioxide nanoparticles dispersed and embedded in the swelled polymer matrix macroparticles over the wavelength range of 280-800 nm was measured using a spectrophotometer. The composition was placed in a 10 mm light pathway quartz cuvette, and the absorbance was measured in view of a reference cuvette containing Cetiol® B, as described in Example 5. Results are presented in FIG. 3B.

As seen in FIG. 3B, the compositions comprising titanium dioxide nanoparticles dispersed and embedded in the swelled biodegradable polymer matrix macroparticles exhibited absorption in the UV range, their efficacy dropping below 50% (i.e. displaying an absorbance of less than 0.301) at about 370 nm at a tested concentration of 0.003 wt. %, indicating that even though the composition contained polymer-embedded TiO₂ nanoparticles at about half the concentration of the non-embedded nanoparticles, its effective absorption in the UV range was maintained.

The absorbance patterns of the titanium dioxide nanoparticles herein exemplified, embedded in the polymeric matrix, though not identical, are highly similar to the patterns measured of the nanoparticles in the liquid oil media (FIG. 3A). Importantly, the substantial lack of red shift in the matrix macroparticles as compared to the oil dispersion indicates that the nanoparticles embedded in the polymer matrix did not agglomerate during the milling step embedding them in the polymer matrix relatively to their original dispersions. Such aggregation of particles would have caused higher scattering and a shift of the absorbance curves towards higher wavelengths, the extent of which may be undesired for particular applications wherein the compositions should be preferably invisible on the target surface. The present findings support that the nanoparticles are well dispersed within the matrix macroparticles of the compositions according to the disclosure.

### Example 7: Absorbance of Zinc Oxide Nanoparticles Dispersed in Oil as Compared to Embedded in Swelled Biodegradable Polymer Matrix Macroparticles

In the present study, Example 5 and Example 6 were repeated with a different pigment. Dispersion Zn-40, containing ZnO as a pigment, Cetiol® RLF as an oil carrier and Pelemol® PHS-8 as a dispersant, as prepared in Example 1, was diluted with its oil carrier to provide ZnO concentrations of 0.005 wt. %, 0.009 wt. %, 0.015 wt. %, 0.02 wt. % and 0.03 wt. %.

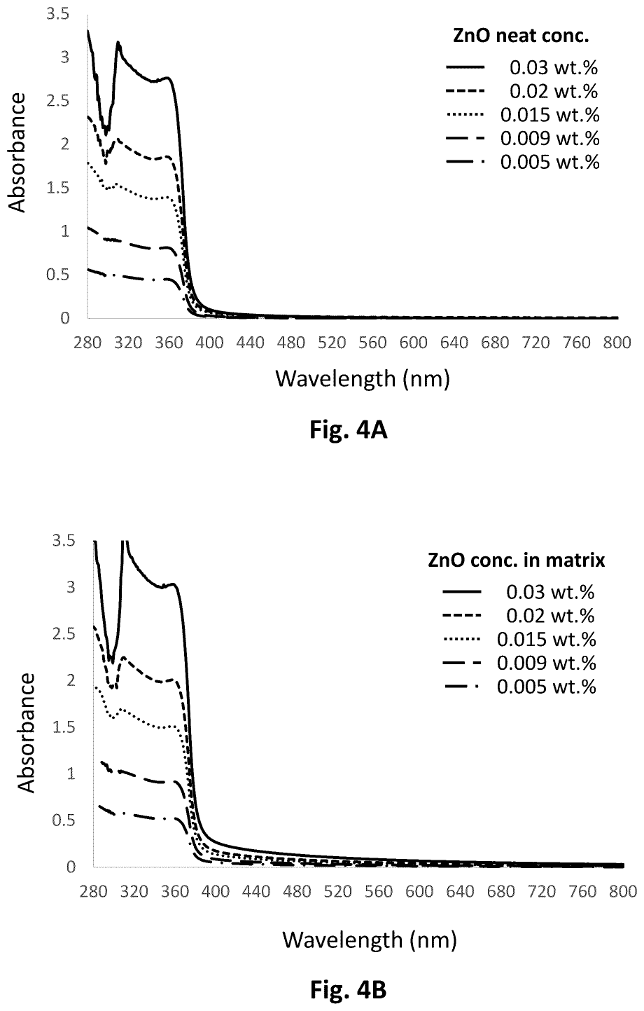

Absorbance of zinc oxide nanoparticles over the wavelength range of 280-800 nm was measured using a spectrophotometer, as described for the TiO₂ nanoparticles in Example 5, and the spectra for the various concentrations are presented in FIG. 4A, where it can be seen that the oil-dispersed zinc oxide nanoparticles absorbed light mainly in the UV range, its efficacy dropping below 50% (i.e. displaying an absorbance of less than 0.301) at about 384-385 nm at its highest tested concentration (0.03 wt. %).

The absorbance of ZnO embedded in swelled biodegradable polymer matrix macroparticles was measured as well, using Comp. 10 (prepared in Example 4, containing ZnO nanoparticles in PHB macroparticles, having a pigment:polymer w/w ratio of 1:1), diluted with Cetiol® RLF to yield samples containing ZnO at the same concentrations as described above.

Absorbance of the zinc oxide nanoparticles dispersed and embedded in the swelled polymer matrix macroparticles over the wavelength range of 280-800 nm was measured as described above, and the spectra for the various concentrations are presented in FIG. 4B, where it can be seen that the polymer-embedded zinc oxide nanoparticles also absorbed light mainly in the UV range, their efficacy dropping below 50% at about 394 nm at a tested concentration of 0.015 wt. %. As previously observed in Example 6 with the nanoparticles of TiO₂, the composition containing polymer-embedded ZnO nanoparticles was as effective as the dispersion at about half the concentration of the non-embedded nanoparticles, had maintained its effective absorption in the UV range. This suggests that surprisingly, the embedment of the pigment particles may relatively increase their efficacy as UV-absorbers, as compared to a same amount of particles freely dispersed and capable of Brownian motion.

The absorbance in the tested range increased as the concentration of the zinc oxide nanoparticles increased for both the oil-dispersed and the polymer embedded samples, and no significant absorption was observed in the visible range, indicating that the nanoparticles are well dispersed within the matrix macroparticles of the present compositions.

### Example 8: Scanning Electron Microscope Studies

The titanium dioxide nanoparticles dispersed and embedded in the swelled biodegradable polymer matrix macroparticles, prepared in Example 4 (Comp. 5, having a pigment concentration of 6 wt. %), were studied by High Resolution Scanning Electron Microscopy (HR-SEM) for their planar dimensions and by FIB-SEM for their thicknesses.

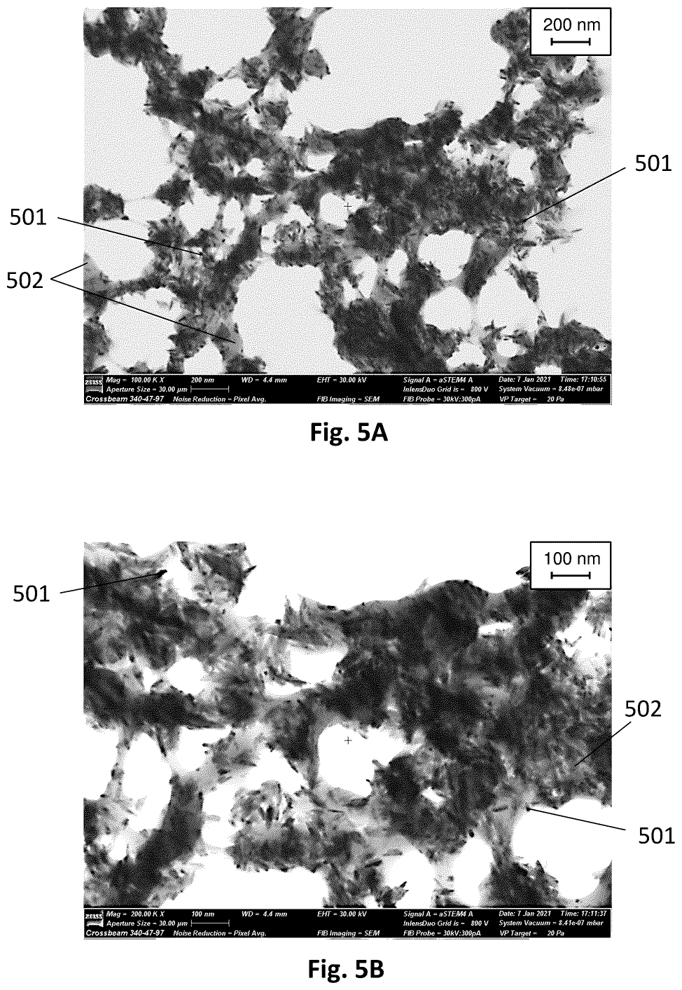

In order to prepare a dry sample suited for SEM analysis, a few drops of Comp. 5 were added to an excess (10 g) of isopropyl alcohol placed in a 20 ml vial. The mixture was mixed by vortex until homogenization, and one drop of the obtained mixture was dried and used for the HR-SEM analysis. FIGS. 5A and 5B show images of nanoparticles 501 of titanium dioxide dispersed and embedded in swelled polymer matrix macroparticles 502, wherein FIG. 5B is a two-fold magnified version of a matrix element shown in FIG. 5A.

As shown in these figures, nanoparticles having a spheroid or rod-like shape with diameters of less than about 100 nm, were obtained. It is believed that the apparent larger clusters in the figures are not aggregates, and are attributed to the presence of individual, separate nanoparticles disposed at different depths across the matrix element, as supported by the absorption of the nanoparticles dispersed and embedded in the polymer matrix being no less than the absorption of a same amount of non-embedded freely dispersed nanoparticles, and being even possibly superior as suggested in Example 6 and Example 7. The good correlation between the dimensions of the inorganic nanoparticles when measured in suspension and observed in dried form confirms the suitability of the above-described method to maintain the nanoparticles well dispersed and sufficiently immobilized in the polymer matrix.

Furthermore, the absence of free particles of titanium oxide as illustrated in either FIG. 5A or FIG. 5B, demonstrates that the pigment particles in the compositions prepared by the methods of the present invention are properly embedded within the swelled biodegradable polymer matrix macroparticles, without any migration out of the matrix. Though not entirely visible in the captured images, the macroparticles displayed flake-like shapes.

All other compositions comprising macroparticles of swelled biodegradable polymeric matrix and dispersed pigment nanoparticles embedded therein, which were produced in Examples 4, displayed a similar appearance, as observed by HR-SEM. Namely, the nanoparticles were well dispersed within the flake-shaped macroparticles and insignificant to null amount of non-embedded pigment particles were observed in the numerous fields of view considered during this study.

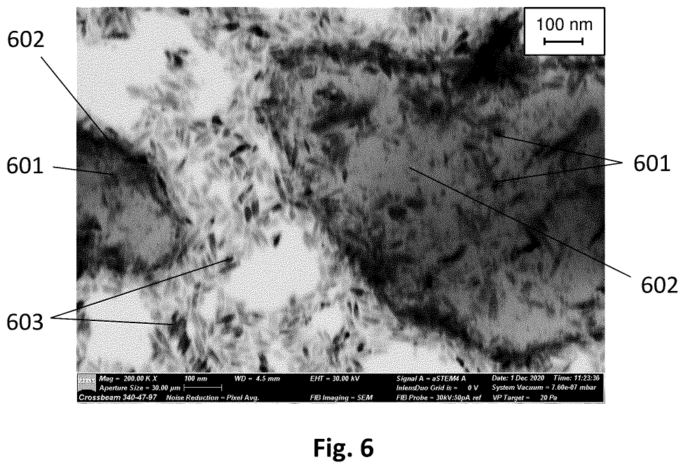

In comparison, FIG. 6 shows polymeric macroparticles 602, embedding pigment nanoparticles 601, where free, non-embedded pigments 603 are widely present outside the polymeric matrix. This result may be undesired and suggest that the materials and/or the method of preparation of the macroparticles used in this comparative control would be unsuitable for the purpose of the present invention.

### Example 9: Absence of Free Pigment Particles in the Composition

The absence of free pigment particles, not being embedded in the macroparticles of swelled biodegradable polymer matrix prepared according to the present teachings can be further demonstrated by an alternative analysis of the composition comprising the macroparticles.

5 g of the composition, such as any one of the compositions prepared in Example 4, are combined with isopropyl alcohol, mixed by vortex and centrifuged. The supernatant is extracted, and the process can be repeated for 2-3 times, to ensure maximal removal of any free pigment particles, if any. The obtained supernatant fractions are combined and then studied by SEM. A predetermined unit area, e.g., 10 μm by 10 μm, is analysed, and the free pigment particles present in the field of view, if any, are counted, to attest to their presence or absence in the composition.

### Example 10: Absorbance Results

Based on the optical absorbance spectra determined in Examples 5 to 7, area under the curve (AUC) was calculated for nanoparticles of TiO₂ (D_{V}50: 44.5 nm) and ZnO (D_{V}50: 41.7 nm) both before and after incorporation in macroparticles of the swelled biodegradable polymer.

Briefly, the absorbance of the nanoparticles, dispersed either in their oil carrier (Ti-55 and Zn-40) or in their polymeric composition (Comp. 5 and Comp. 10), was integrated from 280 nm to 400 nm (AUC₂₈₀₋₄₀₀) and from 280 nm to 700 nm (AUC₂₈₀₋₇₀₀), for each one of the pigment concentrations. The AUC for each pigment concentration in the two ranges of wavelengths is presented in Table 8 below.

TABLE 8

| | AUC₂₈₀₋₄₀₀ | | AUC₂₈₀₋₇₀₀ | |
|---|---|---|---|---|
| TiO₂ | TiO₂ | TiO₂ | TiO₂ | TiO₂ |
| Conc. | nanoparticles | nanoparticles | nanoparticles | nanoparticles |
| [wt. %] | (neat) | in matrix | (neat) | in matrix |
| 0.001 | 43 | 52 | 43 | 66 |
| 0.003 | 131 | 159 | 137 | 204 |
| 0.005 | 220 | 267 | 234 | 343 |
| 0.007 | 329 | 369 | 358 | 476 |

As can be seen from the above table, an exemplary AUC₂₈₀₋₄₀₀ of the 0.007 wt. % TiO₂ nanoparticles dispersed in the biodegradable polymeric matrix is about 12% higher than that of the non-embedded nanoparticles, which demonstrates at least similar absorbance efficacies of the nanoparticles in their non-embedded and embedded states.

These results also demonstrate that the nanoparticles dispersed in the biodegradable polymeric matrix can provide a broad UV protection, as the AUC formed by the UV-absorption of the nanoparticles of TiO₂ in the UV-range of 280 nm to 400 nm (AUC₂₈₀₋₄₀₀) is at least 75% of the AUC formed at the same concentration of TiO₂ in the entire range of 280 nm to 700 nm, which includes the visible range. This preferred absorbance in the UV-range was demonstrated at all concentrations tested and also indicates that the compositions are substantially invisible (transparent).

Similar calculations were performed for ZnO and the results are summarized in Table 9 below.

TABLE 9

| | AUC₂₈₀₋₄₀₀ | | AUC₂₈₀₋₇₀₀ | |
|---|---|---|---|---|
| ZnO | ZnO | ZnO | ZnO | ZnO |
| Conc. | nanoparticles | nanoparticles | nanoparticles | nanoparticles |
| [wt. %] | (neat) | in matrix | (neat) | in matrix |
| 0.005 | 45 | 54 | 46 | 60 |
| 0.009 | 83 | 95 | 84 | 105 |
| 0.015 | 141 | 155 | 146 | 171 |
| 0.030 | 264 | 291 | 269 | 322 |

As can be seen from the above table, the AUC displayed by the embedded ZnO pigment particles is at least comparable to the AUC provided by free pigments at same concentrations. The macroparticles of swelled polymer containing the dispersed nanoparticles of ZnO demonstrated an even more preferred UV absorption, as compared to previous TiO₂, their AUC₂₈₀₋₄₀₀ constituting about 90% of the AUC₂₈₀₋₇₀₀. These results, observed at all tested concentrations, also indicate that the compositions are substantially transparent.

### Example 11: Determination of Critical Wavelength

Based on the optical absorbance spectra determined in Examples 5 to 7, critical wavelength was calculated for the TiO₂ nanoparticles and ZnO nanoparticles both before (Ti-55 and Zn-40) and after incorporation in macroparticles of the swelled biodegradable polymer (Comp. 5 and Comp. 10).

Briefly, in order to quantify the breadth of UV protection, the absorbance of the composition was integrated from 290 nm to 400 nm, the sum reached defining 100% of the total absorbance of the UV-protective composition in the UV region. The wavelength at which the summed absorbance reaches 90% absorbance was defined as the ‘critical wavelength’ which provides a measure of the breadth of UV protection.

The critical wavelength λ_{c} is defined according to the following equation:

$${\int }_{290}^{{λ}_{c}} I g [ 1 l T ( λ ) ] d λ = 0.9 \cdot {\int }_{290}^{400} I g [ 1 l T ( λ ) ] d λ$$

wherein:
λ_{c} is the critical wavelength;
T(λ) is the mean transmittance for each wavelength; and
dλ is the wavelength interval between measurements.

The critical wavelengths so determined for each sample are presented in Table 10 below.

TABLE 10

| | Critical |
|---|---|
| | Wavelength |
| Sample | (nm) |
| TiO₂ nanoparticles dispersed in oil carrier | 350 |
| TiO₂ nanoparticles dispersed and embedded in macroparti- | 370 |
| cles | |
| ZnO nanoparticles dispersed in oil carrier | 366 |
| ZnO nanoparticles dispersed and embedded in macroparti- | 367 |
| cles | |

As can be seen from the above table, the embedment of the dispersed nanoparticles of pigments in swelled polymer matrix macroparticles did not hamper the breadth of UV-protection, as expressed by the similar critical wavelengths before and after said incorporation. Moreover, these results show that the present compositions can provide a broad UV protective effect, their critical wavelengths being of about 370 nm.

### Example 12: Preparation of Compositions Comprising Pigment Particles Embedded in a Mixture of Swelled Biodegradable Polymers

Mixtures of biodegradable polymers were swelled as follows: 22.5 g of polylactic acid (PLA) and 22.5 g of polyhydroxybutyrate (PHB) were placed in a 800 ml glass container and 105 g of lauryl lactate, serving as a swelling agent were added. The container was sealed and placed in an oven, where it was maintained for 24 hours at 100° C., allowing the polymers to swell. The swelled biodegradable polymers (each at a concentration of 15 wt. %, hence at a combined polymer concentration of 30 wt. %) were vigorously manually mixed then allowed to cool to room temperature. This composition can be referred to as SBP30-PLA-PHB.

87.5 g of the Ti-40(GtO-RLF) dispersion of TiO₂ nanoparticles, prepared as described in Example 1, were added to 116.7 g of the swollen polymer mix composition, SBP30-PLA-PHB and 145.8 g of Cetiol® RLF (which was one of the oil carriers used for the pigment dispersion). All were manually mixed to give a combined weight of 350 g and transferred to a zirconia pot of an attritor, loaded with 770 ml of 2 mm zirconium oxide/yttrium stabilized ceramic beads.

The temperature of the pot was maintained at 50° C. by a jacket while the attritor was set to mill the contents of the pot at 700 rpm for 6 hours. A composition according to the present teachings, comprising inorganic nanoparticles of a UV-protective agent dispersed and embedded in the swelled biodegradable polymer matrix macroparticles, was obtained.

This composition is reported in Table 11 as Comp. 12. Other compositions comprising a blend of swellable polymers were prepared according to similar procedures, containing different components in different amounts, and milled under different milling conditions, as specified in the table. The values reported in the table correspond to the concentration of each component in weight percent (wt. %) by total weight of the composition. The particle size distributions (as measured by LS) of the swelled biodegradable polymer matrix macroparticles so prepared are reported in terms of percent of volume of particles. NA means that a particular data is not available.

TABLE 11

| Component | Comp. 12 | Comp. 13 | Comp. 14 |
|---|---|---|---|
| Pigment dispersion | Ti-40(GtO-RLF) | Ti-40(RLF) | Ti-50 |
| Swelled polymer | SBP30-PLA- | SBP30-PLA- | SBP30-PLA- |
| matrix | PHB | PCL | PCL |
| TiO₂ | 10 | 2 | |
| (nanoparticles) | | | |
| TiO₂ | | | 16.7 |
| (microparticles) | | | |
| Pelemol ® PHS-8 | 1 | 0.2 | 1.7 |
| PLA | 5 | 9 | 18 |
| PHB | 5 | | |
| PCL | | 1 | 2 |
| Glyceryl | 10.5 * | | |
| trioctanoate | | | |
| Cetiol ® RLF | 3.5 * | 2.8 * | |
| | 41.7 ** | 61.7 *** | |
| Cetiol ® B | | | 15 * |
| Lauryl lactate | 23.3 ** | 23.3 ** | 46.7 ** |
| Milling conditions | 6 hrs @ | 3 hrs @ | 1 hr @ |
| | 50° C. | 50° C. | 40° C. then |
| | 700 rpm | 700 rpm | 13.5 hrs @ |
| | | | 30° C. |
| | | | 700 rpm |
| Pigment:polymer | 1:1 | 1:5 | 1:1.2 |
| w/w ratio | | | |

PSD measurements

| | | | |
|---|---|---|---|
| Dv10 [μm] | 0.1 | 1.5 | NA |
| Dv50 [μm] | 0.9 | 6.1 | NA |
| Dv90 [μm] | 2.6 | 13.6 | NA |

* originating from the nanoparticles dispersions prepared in Example 1
** originating from the swelled biodegradable polymer compositions
*** originating from an additional amount of oil being added during milling

The swelled biodegradable polymer matrix macroparticles of both Comp. 12 and Comp. 13 displayed appearances, as observed by HR-SEM, similar to that of other compositions previously prepared. Namely, the nanoparticles were well dispersed within the flake-shaped macroparticles comprising mix of swellable polymers and insignificant to null amount of non-embedded pigment nanoparticles were observed in the numerous fields of view considered during this study.

When the pigment particles embedded in the mix of swellable polymers were in the microscopic range (Comp. 14), the flakes were accordingly slightly thicker than flakes embedding nanoparticles. This was to be expected as the thickness of the macroparticles needs to be sufficiently large to envelop the particles. The flakes of Comp. 14 also displayed relatively shorter planar dimensions as compared to flakes containing nanoparticles of pigments, the aspect ratio of macroparticles of swelled polymer containing microparticles of pigments being therefore relatively smaller than the aspect ratio of flake-shaped macroparticles containing nanoparticles of pigments. The TiO₂ microparticles were well dispersed within the polymeric matrix, with insignificant to null amount of non-embedded pigment microparticles, similar to previously obtained compositions.

### Example 13: Alternative Method of Preparation

While in previous examples, the swelling of the biodegradable polymers was separately performed, the pigment particles being added at a later step, in the present study an alternative method of preparing macroparticles of swelled polymers embedding dispersed pigments was demonstrated.

125 g of the Ti-40(RLF) dispersion of TiO₂ nanoparticles, prepared as described in Example 1, were placed in a double planetary mixer vessel. 50 g of unswollen polyhydroxy butyrate and 116.7 g benzyl alcohol were added, and the mixer was operated at a temperature of 80° C. for 2 hours at 20 RPM. The heating was stopped, and the mixture was allowed to cool to room temperature, under continuing mixing.

204.1 g of the obtained mixture was transferred to a zirconia pot of an attritor, loaded with 770 ml of 2 mm zirconium oxide/yttrium stabilized ceramic beads, together with 145.9 g of Cetiol® RLF (which served as oil carrier for the pigment dispersion). The temperature of the pot was maintained at 50° C. by a jacket while the attritor was set to mill the contents of the pot at 700 RPM for 24 hours.

The particle size distribution of the swelled biodegradable polymer matrix macroparticles prepared as herein described, is as follows the macroparticles having a D_{V}10 of 0.8 μm, a D_{V}50 of 1.9 μm, and a D_{V}90 of 4.4 μm. These results are comparable to macroparticles of same composition, prepared by the previous method in which the polymer was pre-swollen separately before being milled with the pigment particles, the milling step being identical to present one (see Comp. 15). The reference macroparticles had a D_{V}10 of 1.0 μm, a D_{V}50 of 2.4 μm and a D_{V}90 of 5.2 μm.

The swelled biodegradable polymer matrix macroparticles prepared as above described displayed an appearance, as observed by HR-SEM, similar to that of other compositions previously prepared. Namely, the nanoparticles were well dispersed within the flake-shaped macroparticles and insignificant to null amount of non-embedded pigment particles were observed in the numerous fields of view considered during this study. These observations support the suitability of this alternative method of preparation.

### Example 14: Effect of Carrier on Absorbance Properties

87.5 g of the Ti-40(RLF) dispersion of TiO₂ nanoparticles, prepared as described in Example 1, were added to 87.5 g of the swollen polymer composition SBP40-PHB, prepared as described in Example 3, and 145.8 g of Cetiol® RLF (which served as oil carrier for the pigment dispersion). They were manually mixed to give a combined weight of 350 g and the mixture was placed in a zirconia pot of an attritor, loaded with 770 ml of 2 mm zirconium oxide/yttrium stabilized ceramic beads.

The temperature of the pot was maintained at 50° C. by a jacket while the attritor was set to mill the contents of the pot at 700 rpm for 24 hours, whereby a composition according to the present teachings, having a pigment content of 10 wt. %, and a pigment:polymer ratio of 1:1 was obtained.

This composition, referred to as Comp. 15, is reported in Table 12. The values reported in the table correspond to the concentration of each component in weight percent (wt. %) by total weight of the composition. The particle size distribution (as measured by LS) of the macroparticles of swelled biodegradable polymer so prepared is provided in terms of percent of volume of particles.

TABLE 12

| Component | Comp. 15 |
|---|---|
| Pigment dispersion | Ti-40(RLF) |
| Swelled polymer matrix | SBP30-PHB |
| TiO₂ | 10 |
| Pelemol ® PHS-8 | 1 |
| ENMAT Y3000 | 10 |
| Cetiol ® RLF | 14 * |
| | 41.7 * |
| Benzyl alcohol | 23.3 |
| Pigment:polymer w/w ratio | 1:1 |

PSD measurements

| | |
|---|---|
| Dv10 [μm] | 1.0 |
| Dv50 [μm] | 2.4 |
| Dv90 [μm] | 5.2 |

* originating from the nanoparticles dispersion prepared in Example 1
** originating from the swelled biodegradable polymer compositions prepared in Example 3
*** originating from an additional amount of oil being added during milling

A portion of Comp. 15 was diluted with Cetiol® RLF to pigment concentrations of 1 wt. %, 2 wt. % and 3 wt. %. Samples of each concentration were applied on quartz slides using a 5 μm wet film applicator, whereby layers having a wet thickness of 5 μm were formed.

In order to study the effect of a volatile carrier, another portion of Comp. 15 was treated to eliminate the oil carrier and replace it with isopropyl alcohol (IPA) as a volatile carrier. The carrier replacement was conducted as follows: 5 g of Comp. 15 were placed in a centrifuge, operated at 9,000 rpm for 15 minutes. Following centrifugation, the supernatant oil carrier was collected with a pipette, weighted, and replaced by the same amount of IPA, the macroparticles were mixed with the new liquid, followed by another separation (under same centrifuging conditions). This liquid replacement step was repeated three times in total, until the flakes were deemed entirely rinsed from their former oil carrier and resuspended in IPA. A portion of the obtained composition was diluted with IPA to pigment concentrations of 1 wt. %, 2 wt. % and 3 wt. %. Samples of each concentration were applied on quartz slides using a 5 μm wet film applicator, whereby layers having a wet thickness of 5 μm were formed. The slides were placed on a hot plate for 10 minutes at a temperature of 200° C., to allow full and rapid elimination of the volatile carrier. The resulting dry thickness of the samples was below 0.5 μm.

The absorbance of the compositions applied onto the slides (either as 5 μm wet layers for the macroparticles suspended in an oil carrier or as 0.5 μm dry layers for the macroparticles devoid of their former volatile carrier) was measured using a spectrophotometer over a range spanning from 280 nm to 700 nm. The values obtained with a reference clean slide were subtracted.

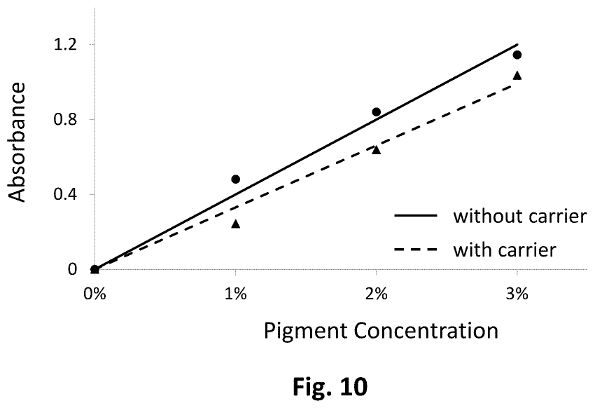

The absorbance results for the samples at a selected wavelength of 308 nm (wherein a maximal erythemal response is generally obtained) are presented in FIG. 10, trendlines being added for each type of source composition. A broken line indicates the trend of the absorbance results displayed by the slides “with carrier” still containing the oil carrier Cetiol® RLF, whereas a full line indicates the trend of absorbance results for macroparticles of Comp. 15 “without carrier”, the original carrier having been replaced by a volatile one to enable its elimination.

As can be seen in FIG. 10, the samples being devoid of carrier generally displayed a higher absorbance as compared to the compositions wherein the oil carrier was retained, at all pigment concentrations tested. Conversely, these results show that for any desired level of absorbance, for illustration for a value of 1.2 where more than 99% of the wavelength is absorbed, the amount of pigment necessary to achieve this effect would be relatively lower if the composition is relatively dry as compared to a composition further including a carrier. In the present illustration, while 3% of pigment would provide for an absorbance of 1.2 in the dry composition, 3.5% of pigments may be required to achieve a similar effect in the wet composition. While the illustrated differences may seem minor in terms of quantities, they may nevertheless provide a commercially significant advantage. For instance, an effective composition requiring less pigments to achieve a desired effect, may also necessitate less dispersants, shorter milling durations, and so on. Regardless of the advantages dry compositions may have over wet ones, the present results support that macroparticles prepared by the present methods could effectively block UV radiation with or without a liquid media, further supporting that the reported absorbances are entirely stemming from the pigments dispersed and embedded in the macroparticles of swelled biodegradable polymer.

### Example 15: Effect of HLB of Liquid Agents on Swelling

The ability of liquids having different calculated HLB values to swell biodegradable polymers was assessed. The HLB values were calculated according to the Davies method, as previously described, using the following calculation method: HLB=7+Σ(hydrophilic group values)+Σ(lipophilic group values).

The liquids tested as prospective swelling agents were isoparaffin (Isopar™ L), being relatively non-polar with a calculated HLB value of 0.8-1.8, as compared to relatively polar liquids, namely dibutyl adipate (Cetiol® B) and benzyl alcohol, having calculated HLB values of 6 and 6.7, respectively.

Known amounts of beads of biodegradable polymers, weighing between 2 and 5.1 g, were separately placed in 20 ml glass containers, and candidate swelling agents were added to fill the containers to their top. The containers were then sealed and placed in an oven for an incubation period of 4 days at 60° C. to enable swelling, if any, of the tested polymers. Following incubation, the containers were taken out of the oven and allowed to cool down for 15 minutes at room temperature. The liquids were removed by filtration and the resulting polymeric beads were washed with IPA and allowed to dry. The polymers were then weighted.

The difference in the weight of the tested polymers before and after incubation with the prospective swelling agents is presented in percentage of the initial values in Table 13. A weight gain of at least 5 wt. % is considered satisfactory and liquids providing such outcomes in the present study are considered suitable swelling agents of the respective biodegradable polymer.

TABLE 13

| | Polymers | | | | |
|---|---|---|---|---|---|
| Swelling agents | PLA | PCL | PHB | PHBV | PBSA |
| Isopar ™ L | −0.6% | +0.1% | −0.1% | −0.1% | −0.1% |
| Cetiol ® B | +29.6% | +853.4% | NA | NA | NA |
| Benzyl alcohol | NA | NA | +27.1% | +16.3% | +789.8% |

As can be seen from the above table, the non-polar agent Isopar™ L, being an isoparaffin having a chain of 11-13 carbon atoms and a calculated HLB value of less than 3, failed to properly swell any of the biodegradable polymers tested, whereas Cetiol® B and benzyl alcohol, both being more polar and having a calculated HLB value of more than 3, successfully swelled these polymers, hence qualifying as swelling agents for the present teachings. For reference, Isopar™ L is a potent swelling agent when similarly tested with non-biodegradable polymers, providing for a weight gain of at least 20 wt. % with such polymers, as disclosed in Table 3 of U.S. Pat. No. 10,617,610.

It is appreciated that certain features of the disclosure, which are, for clarity, described in the context of separate embodiments or a particular aspect, may also be provided in combination in a single embodiment, or may apply to any other aspect or embodiments of said other aspects, unless incompatible therewith. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the present disclosure has been described with respect to various specific embodiments presented thereof for the sake of illustration only, such specifically disclosed embodiments should not be considered limiting. Many other alternatives, modifications and variations of such embodiments will occur to those skilled in the art based upon Applicant's disclosure herein. Accordingly, it is intended to embrace all such alternatives, modifications and variations and to be bound only by the spirit and scope of the disclosure and any change which come within their meaning and range of equivalency.

In the description and claims of the present disclosure, each of the verbs “comprise”, “include” and “have”, and conjugates thereof, are used to indicate that the object or objects of the verb are not necessarily a complete listing of features, members, steps, components, elements or parts of the subject or subjects of the verb. Yet, it is contemplated that the compositions of the present teachings also consist essentially of, or consist of, the recited components, and that the methods of the present teachings also consist essentially of, or consist of, the recited process steps.

As used herein, the singular form “a”, “an” and “the” include plural references and mean “at least one” or “one or more” unless the context clearly dictates otherwise. At least one of A and B is intended to mean either A or B, and may mean, in some embodiments, A and B.

Unless otherwise stated, the use of the expression “and/or” between the last two members of a list of options for selection indicates that a selection of one or more of the listed options is appropriate and may be made.

Unless otherwise stated, when the outer bounds of a range with respect to a feature of an embodiment of the present technology are noted in the disclosure, it should be understood that in the embodiment, the possible values of the feature may include the noted outer bounds as well as values in between the noted outer bounds.

As used herein, unless otherwise stated, adjectives such as “substantially”, “approximately” and “about” that modify a condition or relationship characteristic of a feature or features of an embodiment of the present technology, are to be understood to mean that the condition or characteristic is defined to within tolerances that are acceptable for operation of the embodiment for an application for which it is intended, or within variations expected from the measurement being performed and/or from the measuring instrument being used. When the term “about” and “approximately” precedes a numerical value, it is intended to indicate +/−15%, or +/−10%, or even only +1-5%, and in some instances the precise value. Furthermore, unless otherwise stated, the terms (e.g., numbers) used in this disclosure, even without such adjectives, should be construed as having tolerances which may depart from the precise meaning of the relevant term but would enable the invention or the relevant portion thereof to operate and function as described, and as understood by a person skilled in the art.

While this disclosure has been described in terms of certain embodiments and generally associated methods, alterations and permutations of the embodiments and methods will be apparent to those skilled in the art. The present disclosure is to be understood as not limited by the specific embodiments described herein. Therefore, the terms “as described herein”, should be more broadly understood to mean “substantially as described herein” or “essentially as described herein” including, when applicable, as shown in the examples and/or illustrated in the drawings.

To the extent necessary to understand or complete the disclosure of the present disclosure, all publications, patents, and patent applications mentioned herein, including in particular the applications of the Applicant, are expressly incorporated by reference in their entirety by reference as is fully set forth herein.

Certain marks referenced herein may be common law or registered trademarks of third parties. Use of these marks is by way of example and shall not be construed as descriptive or limit the scope of this disclosure to material associated only with such marks.

Citation or identification of any reference in this application shall not be construed as an admission that such reference is available as prior art to the disclosure.

1. A composition comprising:
a) macroparticles of swelled polymer matrix comprising a swellable thermoplastic biodegradable polymer swelled with at least one non-aqueous polar swelling agent having a calculated Hydrophilic-Lipophilic Balance (HLB) value of at least about 3; and
b) a plurality of pigment particles dispersed and embedded in the macroparticles of swelled biodegradable polymer matrix;
the swellable thermoplastic biodegradable polymer having at least one of a melting temperature (Tm), a softening temperature (Ts) and a glass transition temperature (Tg) of 200° C. or less.

2. The composition as claimed in claim 1, wherein at least a part of the plurality of pigment particles includes pigment particles having a dispersant associated therewith, the dispersant being oil-soluble and optionally having a HLB value of no more than 9, no more than 6, or no more than 3.

3. The composition as claimed in claim 1, wherein the composition is substantially devoid of free pigment particles non-embedded in the macroparticles of the swelled biodegradable polymer matrix, a ratio between a number of free non-embedded pigment particles and a number of macroparticles of swelled biodegradable polymer matrix being at most 10:1, at most 5:1, a most 2:1, at most 1:1, at most 1:2, at most 1:5, at most 1:10, or at most 1:20, as measured per unit area of a field of view of a microscope.

4. The composition as claimed in claim 1, wherein the swellable thermoplastic biodegradable polymer is selected from:
I) synthetic biodegradable polymers selected from a group consisting of polyesters, polyamides, polyurethanes, polyureas, poly(amide-enamine)s, polyanhydrides, enantiomers, and copolymers thereof;
II) natural biodegradable polymers selected from a group consisting of polysaccharides, polypeptides and resins;
and combinations thereof.

5. The composition as claimed in claim 4, wherein the swellable thermoplastic biodegradable polymer is a synthetic biodegradable polyester selected from a group comprising poly(lactic acid), poly(lactide-co-glycolide), polycaprolactone, poly(glycolic acid), polyhydroxybutyrate, poly(3-hydroxybutyrate-co-3-hydroxyvalerate), poly(butylene succinate), poly(butylene succinate-co-adipate), poly(ethylene succinate), poly(trimethylene carbonate), poly(propylene carbonate), poly[oligo (tetramethylene succinate)-co(tetramethylenecarbonate), poly(p-dioxanone); poly(ethylene terephtalate), poly(butylene adipate-co-terephtalate), copolymers and enantiomers thereof; or a natural biodegradable polymer selected from a group comprising shellac, thermoplastic starch (TPS), cellulose, chitin, chitosan, fibers, gums, pullulan, corn zein, wheat gluten, soy protein, collagen, gelatin, casein, caseinates, whey proteins, and derivatives thereof.

6. The composition as claimed in claim 4, wherein the swellable thermoplastic biodegradable polymer is one or more enantiomer or copolymer of poly(lactic acid), poly(lactic-co-glycolic acid), polyhydroxybutyrate, poly(3-hydroxybutyrate-co-3-hydroxyvalerate), poly(butylene succinate), poly(butylene succinate-co-adipate), polycaprolactone, shellac, or gum rosin.

7. The composition as claimed in claim 1, wherein at least one of the at least one non-aqueous polar swelling agent has a calculated HLB value of 4 or more, 5 or more, or 6 or more; and optionally of 25 or less, 20 or less, 15 or less.

8. The composition as claimed in claim 1, wherein the at least one non-aqueous polar swelling agent having a calculated HLB value of 3 or more is selected from a group consisting of saturated or unsaturated acid mono or diesters, fatty acid ester, cyclic organic esters, aromatic alcohols, glycols, polyols, fatty alcohols, aldehydes, and ethers.

9. The composition as claimed in claim 8, wherein the at least one non-aqueous polar swelling agent is selected from a group comprising 2-ethylhexyl lactate, acetyl tributyl citrate, acetyl triethyl hexyl citrate, acetyl triethyl citrate, allyl hexanoate, benzyl alcohol, benzyl benzoate, butyl butyryllactate, butyl lactate, caprylyl caprylate, caprylyl caprate, cinnamaldehyde, dibutyl adipate, dibutyl maleate, dibutyl sebacate, diethyl succinate, diethylene glycol diethyl ether, dimethyl glutarate, dimethyl isosorbide, dimethyl maleate, dimethyl methylglutarate, ethyl lactate, gamma decalactone, glyceryl trioctanoate, lactic acid isoamyl ester, lauryl lactate, 1-menthyl lactate, menthalactone, n-pentyl benzoate, PEG-6 caprylic/capric glycerides, triacetin, triethyl citrate and and undecanoic lactone.

10. The composition as claimed in claim 1, wherein the pigment particles are nanoparticles of an organic or inorganic pigment adapted to provide at least a UV-protective effect, the pigment nanoparticles having a maximal particle size (D_{V}99) of up to 250 nm, up to 200 nm, up to 150 nm, up to 100 nm, up to 90 nm, up to 80 nm, up to 70 nm or up to 60 nm; and optionally a D_{V}10 of at least 2 nm, at least 5 nm, at least 10 nm, at least 15 nm, or at least 20 nm.

11. The composition as claimed in claim 10, wherein the pigment nanoparticles are of an inorganic UV-absorbing agent being a doped or undoped metal oxide selected from a group comprising aluminium oxide, barium titanate, bismuth oxide, bismuth vanadate, bismuth titanate, bismuth oxybromide, bismuth oxychloride, cerium oxide, cobalt oxide, cobalt titanate, copper oxide, iron oxide, magnesium aluminum oxide, manganese dioxide, manganese ferrite, nickel antimony, manganese vanadium oxide, titanium dioxide, zinc oxide, zinc titanate, zirconium oxide, zirconium titanate, and combinations thereof.

12. The composition as claimed in claim 1, wherein the pigment particles are microparticles of an organic or inorganic pigment adapted to provide at least a coloring effect, the pigment microparticles having a maximal particle size (D_{V}99) of up to 5 μm, up to 3 μm, or up to 1 μm; and optionally of at least 0.25 μm, at least 0.4 μm, at least 0.5 μm or at least 0.6 μm.

13. The composition as claimed in claim 1, wherein the macroparticles of swelled biodegradable polymer matrix are flake-shaped, each flake having a flake length (Lf), a flake width (Wf), and a flake thickness (Tf), and a dimensionless flake aspect ratio (Rf) defined by:
Rf=(Lf*Wf)/(Tf)²
wherein the flakes of a representative group containing at least 10, at least 20, or at least 50 flake-shaped macroparticles of swelled biodegradable polymer matrix fulfil one or more of the following structural features:
a—a flake length (Lf) of said flakes, and/or average thereof, is at most 50 μm, at most 25 μm, at most 10 μm, or at most 5 μm;
b—a flake length (Lf) of said flakes, and/or average thereof, is at least 0.25 μm, at least 0.5 μm, at least 1 μm, at least 1.5 μm, or at least 2 μm;
c—a flake width (Wf) of said flakes, and/or average thereof, is at most 40 μm, at most 20 μm, at most 10 μm, or at most 5 μm;
d—a flake width (Wf) of said flakes, and/or average thereof, is at least 0.2 μm, at least 0.4 μm, at least 0.8 μm, or at least 1 μm;
e—a flake thickness (Tf) of said flakes, and/or average thereof, is at most 1 μm, at most 500 nm, at most 400 nm, at most 350 nm, at most 300 nm, at most 275 nm, at most 250 nm, or at most 225 nm;
f—a flake thickness (Tf) of said flakes, and/or average thereof, is at least 50 nm, at least 75 nm, at least 100 nm, at least 125 nm, at least 150 nm, or at least 175 nm;
g—the aspect ratio (Rf) of said flakes, and/or average thereof, is at least 5, at least 10, at least 15, at least 20, at least 25, at least 30, at least 50, at least 100, at least 150, at least 250, or at least 500;
h—the aspect ratio (Rf) of said flakes, and/or average thereof, is at most 2,000, at most 1,500, or at most 1,000;
i—an aspect ratio (ASP) between a flake length (Lf) of said flakes, and/or average thereof, and a flake thickness (Tf) of said flakes, and/or average thereof, ASP=Lf/Tf, is 1,000:1 or less, 500:1 or less, 100:1 or less, 75:1 or less, 50:1 or less, 25:1 or less, or 10:1 or less;
j—an aspect ratio (ASP) between a flake length (Lf) of said flakes, and/or average thereof, and a flake thickness (Tf) of said flakes, and/or average thereof, ASP=Lf/Tf, is at least 3:1, at least 5:1, at least 10:1, at least 25:1, at least 40:1, or at least 70:1; and
k—an aspect ratio (ASP) between a flake length (Lf) of said flakes, and/or average thereof, and a flake thickness (Tf) of said flakes, and/or average thereof, ASP=Lf/Tf, is within a range of 3:1 to 500:1, 4:1 to 500:1, 8:1 to 500:1, 20:1 to 500:1, 20:1 to 300:1, 20:1 to 250:1, 20:1 to 200:1, or 20:1 to 100:1.

14. The composition as claimed in claim 1, wherein the pigment particles are present in the macroparticles of swelled biodegradable polymer matrix at a weight concentration in a range from 0.01 to 400 wt. %, from 0.05 to 300 wt. %, from 0.1 to 200 wt. %, from 0.1 to 100 wt. %, from 0.1 to 80 wt. %, from 0.1 to 60 wt. %, from 0.1 to 40 wt. %, from 0.3 to 30 wt. %, or from 0.5 to 25 wt. %, by weight of the swellable thermoplastic biodegradable polymer.

15. The composition as claimed in claim 1, the composition being a UV-protective composition having at least one of:
i—a critical wavelength of at least 366 nm, at least 367 nm, at least 368 nm, at least 369 nm, or at least 370 nm; and
ii—an area under the curve (AUC) in a plot of absorption as a function of wavelength such that the AUC in the range of 280 nm to 400 nm (AUC₂₈₀₋₄₀₀) is at least 75%, at least 85% or at least 95% of the AUC range of 280 nm to 700 nm (AUC₂₈₀₋₇₀₀);
said critical wavelength and/or AUC being optionally determined at a pigment concentration of at least 0.001 wt. % per weight of sample.

16. A method of preparing a composition comprising macroparticles of swelled thermoplastic biodegradable polymer matrix including a plurality of pigment particles dispersed and embedded therein, the method comprising:
(a) swelling a thermoplastic biodegradable polymer having at least one of a melting temperature (Tm), a softening temperature (Ts) and a glass transition temperature (Tg) of 200° C. or less with at least one non-aqueous polar swelling agent having a calculated Hydrophilic-Lipophilic Balance (HLB) value of at least about 3, the swelling being performed at at least one swelling temperature and/or under at least one swelling pressure for a swelling period so as to obtain a swelled polymer matrix including at least 5 wt. % of swelling agent per weight of polymer; and
(b) milling the swelled polymer matrix in presence of a plurality of pigment particles, the milling being performed at at least one milling temperature for a milling period so as to size reduce and shape the matrix of swelled thermoplastic biodegradable polymer into macroparticles of swelled polymer matrix, whilst dispersing and embedding the pigment particles in the macroparticles, whereby substantially all pigment particles are embedded in the macroparticles of the swelled biodegradable polymer matrix, a ratio between a number of free non-embedded pigment particles and a number of macroparticles of swelled biodegradable polymer matrix being at most 10:1, at most 5:1, a most 2:1, at most 1:1, at most 1:2, at most 1:5, at most 1:10, or at most 1:20, as measured per unit area of a field of view of a microscope.

17. The method as claimed in claim 16, wherein:
i) the at least one swelling temperature is 30° C. or more, 40° C. or more, 50° C. or more, 60° C. or more, 75° C. or more, or 90° C. or more, the swelling temperature not exceeding 200° C.; and the at least one swelling pressure is 100 kiloPascal (kPa) or more, 150 kPa or more, 200 kPa or more, 250 kPa or more, or 300 kPa or more, the swelling pressure not exceeding 10 megaPascal (MPa); the swelling being performed for a swelling period of at least 10 minutes optionally under ongoing agitation of the swelling polymer matrix in the at least one non-aqueous polar swelling agent;
ii) the plurality of pigment particles present during milling are pigment particles dispersed in a non-aqueous carrier being same or different than the one or more non-aqueous polar swelling agents swelling the polymer; the dispersion being performed for a dispersing period so that the dispersed pigment particles have a maximal particle size (D_{V}99) not exceeding 5 μm prior to milling; and
iii) the at least one milling temperature is lower than a Tm of the swelled polymer or is lower than a temperature being 20° C. greater than a Ts of the swelled polymer; and the milling involves shear force and impact force, the milling being optionally performed for at least 30 minutes.

18. The method as claimed in claim 16, further comprising providing a dispersant able to associate with at least a part of the plurality of pigments, the dispersant:
I) being present in at least one of the at least one non-aqueous polar swelling agent and/or the non-aqueous carrier at a weight per weight ratio of dispersant to pigment particles between 2:1 and 1:100, between 1:1 and 1:100, between 1:1 and 1:90, between 1:1 and 1:70, or between 1:3 and 1:50; and
II) being oil-soluble, optionally having a HLB value of no more than 9, no more than 6, or no more than 3; and optionally contains an acidic moiety.

19. The method as claimed in claim 16, wherein:
A—the swellable thermoplastic biodegradable polymer is selected from a group consisting of polyesters, polyamides, polyurethanes, polyureas, poly(amide-enamine)s, polyanhydrides, polysaccharides, polypeptides, resins, enantiomers, copolymers and combinations thereof;
B—the at least one non-aqueous polar swelling agent having a calculated HLB value of 3 or more is selected from a group consisting of saturated or unsaturated acid mono or diesters, fatty acid esters, cyclic organic esters, aromatic alcohols, glycols, polyols, fatty alcohols, aldehydes, and ethers;
C—the pigment particles embedded in the macroparticles of the swelled biodegradable polymer matrix are: (C1) nanoparticles of an organic or inorganic pigment adapted to provide at least a UV-protective effect, the pigment nanoparticles having a maximal particle size (D_{V}99) between 5 nm and 250 nm; and/or (C2) microparticles of an organic or inorganic pigment adapted to provide at least a coloring effect, the pigment microparticles having a D_{V}99 between 0.25 μm and 5 μm; and
D—the non-aqueous carrier is selected from a group consisting of saturated or unsaturated acid mono or diesters, fatty acid esters, cyclic organic esters, C₁₀-C₃₀ hydrocarbons, aromatic alcohols, glycols, polyols, fatty alcohols, aldehydes, and ethers, the carrier optionally being non-polar and having a calculated HLB value of less than 3.

20. The method as claimed in claim 16, wherein the macroparticles of swelled biodegradable polymer matrix are flake-shaped, each flake having a flake length (Lf), a flake width (Wf), and a flake thickness (Tf), and a dimensionless flake aspect ratio (Rf) defined by:
Rf=(Lf*Wf)/(Tf)²
wherein the flakes of a representative group containing at least 10, at least 20, or at least 50 flake-shaped macroparticles of swelled biodegradable polymer matrix fulfil one or more of the following structural features:
a—a flake length (Lf) of said flakes, and/or average thereof, is at most 50 μm, at most 25 μm, at most 10 μm, or at most 5 μm;
b—a flake length (Lf) of said flakes, and/or average thereof, is at least 0.25 μm, at least 0.5 μm, at least 1 μm, at least 1.5 μm, or at least 2 μm;
c—a flake width (Wf) of said flakes, and/or average thereof, is at most 40 μm, at most 20 μm, at most 10 μm, or at most 5 μm;
d—a flake width (Wf) of said flakes, and/or average thereof, is at least 0.2 μm, at least 0.4 μm, at least 0.8 μm, or at least 1 μm;
e—a flake thickness (Tf) of said flakes, and/or average thereof, is at most 1 μm, at most 500 nm, at most 400 nm, at most 350 nm, at most 300 nm, at most 275 nm, at most 250 nm, or at most 225 nm;
f—a flake thickness (Tf) of said flakes, and/or average thereof, is at least 50 nm, at least 75 nm, at least 100 nm, at least 125 nm, at least 150 nm, or at least 175 nm;
g—the aspect ratio (Rf) of said flakes, and/or average thereof, is at least 5, at least 10, at least 15, at least 20, at least 25, at least 30, at least 50, at least 100, at least 150, at least 250, or at least 500;
h—the aspect ratio (Rf) of said flakes, and/or average thereof, is at most 2,000, at most 1,500, or at most 1,000;
i—an aspect ratio (ASP) between a flake length (Lf) of said flakes, and/or average thereof, and a flake thickness (Tf) of said flakes, and/or average thereof, ASP=Lf/Tf, is 1,000:1 or less, 500:1 or less, 100:1 or less, 75:1 or less, 50:1 or less, 25:1 or less, or 10:1 or less;
j—an aspect ratio (ASP) between a flake length (Lf) of said flakes, and/or average thereof, and a flake thickness (Tf) of said flakes, and/or average thereof, ASP=Lf/Tf, is at least 3:1, at least 5:1, at least 10:1, at least 25:1, at least 40:1, or at least 70:1; and
k—an aspect ratio (ASP) between a flake length (Lf) of said flakes, and/or average thereof, and a flake thickness (Tf) of said flakes, and/or average thereof, ASP=Lf/Tf, is within a range of 3:1 to 500:1, 4:1 to 500:1, 8:1 to 500:1, 20:1 to 500:1, 20:1 to 300:1, 20:1 to 250:1, 20:1 to 200:1, or 20:1 to 100:1.
